(19) United States
(12) Patent Application Publication
(10) Pub. No.: US 2026/0232797 A1
(43) Pub. Date: Aug. 13, 2026

(54) MODULAR CHIMERIC ANTIGEN RECEPTOR

(71) Applicant: INNATE PHARMA, MARSEILLE (FR)

(72) Inventors: STÉPHANIE CORNEN, CARNOUX EN PROVENCE (FR); LAURENT GAUTHIER, MARSEILLE (FR); NICOLAS JARMUZYNSKI, MARSEILLE (FR); YANNIS MOREL, MARSEILLE (FR); ERIC VIVIER, CASSIS (FR)

(21) Appl. No.: 19/471,780

(22) PCT Filed: Apr. 2, 2024
(86) PCT No.: PCT/EP2024/058922

§ 371 Date: Oct. 2, 2025

Related U.S. Application Data
(60) Provisional application No. 63/494,012, filed on Apr. 4, 2023.

(51) Int. Cl. A61K 40/11; A61K 40/15; A61K 40/31; A61P 35/00; C07K 16/28

(52) U.S. Cl. CPC A61K 40/11; A61K 40/15; A61K 40/31; A61P 35/00; C07K 16/2803; C07K 16/2887; A61K 2239/15; A61K 2239/17; A61K 2239/21; A61K 2239/22; C07K 2317/31

(57) ABSTRACT

The invention relates to chimeric activating receptor (CAR) signaling modules. The signaling modules, when introduced into effector cells, permit the cells to lyse target cells such as cancer cells. The modules and the cells comprising them have utility in the treatment of disease, notably cancer or infectious disease, and can be used advantageously in treatment of disease in combination with multispecific molecules that bind the target cell and the CAR signaling module.

### CROSS-REFERENCE TO RELATED APPLICATIONS

This application claims the benefit of U.S. Provisional Application No. 63/494,012 filed 4 Apr. 2023, the disclosure of which is incorporated herein by reference in its entirety; including any drawings and sequence listings.

### REFERENCE TO THE SEQUENCE LISTING

The present application is being filed along with a Sequence Listing in electronic format. The Sequence Listing is provided as a file entitled “NKp46-20 PCT.xml”, created 2 Apr. 2024, which is 232 KB in size. The information in the electronic format of the Sequence Listing is incorporated herein by reference in its entirety.

### FIELD OF THE INVENTION

The invention relates to chimeric activating receptor (CAR) signaling modules. The signaling modules, when introduced into effector cells, permit the cells to lyse target cells such as a cancer cells. The modules and the cells comprising them have utility in the treatment of disease, notably cancer or infectious disease, and can be used advantageously in treatment of disease in in combination with multispecific molecules that bind the target cell and the CAR signaling module.

### BACKGROUND

Natural killer (NK) cells are a subpopulation of lymphocytes that are involved in non-conventional immunity. NK cells provide an efficient immunosurveillance mechanism by which undesired cells such as tumor or virally-infected cells can be eliminated. Characteristics and biological properties of NK cells include the expression of surface antigens including CD16, CD56 and/or CD57, the absence of the α/β or γ/δ TCR complex on the cell surface, the ability to bind to and kill cells in a MHC-unrestrictive manner and in particular cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate the immune response. Interest has also focused on natural killer (NK) cells due to their potential anti-tumor properties.

Multispecific natural killer cell engager (NKCEs) proteins that bind to NK cells and to tumor cells have been developed in order to induce NK-cell-mediated cytotoxicity toward tumor cells. WO2015/197593, WO2016/207278 and WO2017/114694 for example report NKCE protein configurations and NKp46-binding sequences for the production of multispecific NKCE proteins with the ability to induce NK cell mediated lysis of tumor cells and other target cells of interest. In particular, Gauthier et al. (Cell; 2019; 177, 1701-1713) reports trifunctional natural killer (NK) engagers, or NKCEs, targeting two activating receptors, NKp46 and CD16, on NK cells, and a tumor antigen on cancer cells, selected from CD19 or CD20 to target Daudi human B cell lymphoma cells, and EGFR to target A549 human lung carcinoma. WO2022/200525 discloses NKCEs that bind to NKp46, CD122, and optionally further CD16A, on NK cells, as well as to an antigen of interest on a target cells (Demaria et al. (2022), Cell report medicine 3(10): 100783). Such NKCEs increase NK cell cytotoxicity toward the target cell expressing the antigen of interest.

There remains however, a need to develop improved approaches to improve elimination of cancer cells by the immune system to treat or prevent cancer.

### SUMMARY OF THE INVENTION

Disclosed herein is an approach to enhance the cytotoxic (e.g. anti-tumor) activity of immune effector cells (e.g. T cells, NK cells). Disclosed herein is also an approach to enhance the cytotoxic activity of multispecific proteins that bind via a first binding moiety to a surface marker expressed by NK cells (e.g. an ABD that binds to NKp46 on the surface of the cells) and via a second binding moiety to an antigen of interest on the surface of target cells (e.g. a tumor antigen on tumor cells). This approach arises from the combined use (e.g. administration) of a NKCE and engineered cells (e.g. NK cells) that express a chimeric activating receptor, wherein the chimeric activating receptor binds to the NKCE. The NKCE, when used together with the chimeric activating receptor (CAR), acts as a modular chimeric activating receptor, wherein the NKCE moiety acts as a potentiator module (e.g. targeting a tumor cell) and wherein the chimeric activating receptor moiety acts as a signaling module capable of inducing potent activation of the immune cell expressing it. This approach allows the recruitment of the desired immune cells (e.g. engineered T cells, engineered NK cells) to a disease site (e.g. tumor cell, solid tumor, lesion, tumor tissue) and the activation of the immune cells when the CAR potentiator module (or NKCEs) recognize a tumor antigen express by a tumor cell.

In one embodiment, the present disclosure provides a protein (referred to as the chimeric activating receptor (CAR) signaling module) comprising:
(i) an extracellular domain (ECD) of an NKp46 protein or fragment thereof; (ii) a transmembrane domain; and (iii) an intracellular signaling domain. The protein can optionally be specified as being a recombinant and/or chimeric protein comprising protein domains from at least two different. The transmembrane domain and/or intracellular signaling domain can optionally be specified as being from a cell surface receptor other than the protein from which the ECD is derived. In one embodiment, provided is a chimeric activating receptor (CAR) signaling module comprising: (i) an extracellular domain of a human NKp46 protein or fragment thereof; (ii) a transmembrane domain; and (iii) an intracellular signaling domain, wherein (ii) and/or (iii) are from a protein other than NKp46. Also provided are cells (e.g. immune cells, NK cells, T cells) made to express the CAR signaling module at their surface. The cells are particularly adapted for combined in vivo or in vitro use (e.g. for cancer treatment, tumor cell killing) with a potentiator module (e.g. an NKCE) that binds to the extracellular domain of the NKp46 protein and to tumor cells.

In some embodiments, the CAR signaling module comprises an extracellular domain of a NKp46 protein or a fragment thereof that binds to an anti-NKp46 antigen binding domain. In one embodiment, the CAR signaling module comprises an ECD of a NKp46 protein or a fragment thereof that binds to an anti-NKp46 antigen binding domain (or an antibody comprising such) comprising a heavy chain CDR1 of SEQ ID NO: 49, a heavy chain CDR2 of SEQ ID NO: 52, a heavy chain CDR3 of SEQ ID NO: 55, a light chain CDR1 of SEQ ID NO: 97, a light chain CDR2 of SEQ ID NO: 100, a light chain CDR3 of SEQ ID NO: 101. In some embodiments, the CAR signaling module comprises an extracellular domain of a NKp46 protein or fragment thereof that binds to an antibody comprising a heavy chain variable region (VH) of SEQ ID NOS: 146 or 147, and a light chain variable region of SEQ ID NO: 148. In some embodiments, the CAR signaling module comprises an extracellular domain of an NKp46 protein that comprises the amino acid sequence of SEQ ID NO: 4 or a sequence at least 80%, 90%, 95% or 98% identical thereto, or a functional fragment thereof (e.g. a fragment comprising a contiguous sequence of at least 20, 40, 50, 100 or more amino acid residues of the NKp46 sequence). In some embodiments, the CAR signaling module comprises a transmembrane domain from a protein selected from the group consisting of: CD3 zeta, 2B4, DAP10, DAP12, 4-1EE, CD28, CD8 alpha and OX40. In some embodiments, the CAR signaling module comprises an intracellular signaling domain comprising a functional signaling domain from CD3 zeta. In some embodiments, the CAR signaling module comprises an intracellular signaling domain that further comprises at least one costimulatory signaling domain comprising a functional domain of a protein selected from the group consisting of 2B4, DAP10, DAP12, 4-1 BB, CD28 and OX40.

In some embodiments, the CAR signaling module comprises an intracellular signaling domain that comprises a functional signaling domain from CD3 zeta comprising an amino acid sequence of SEQ ID NO: 18. In some embodiments, the CAR signaling module comprises an intracellular signaling domain that comprises a costimulatory signaling domain of 2B4 comprising an amino acid sequence of SEQ ID NO: 19. In some embodiments, the CAR signaling module comprises an intracellular signaling domain that comprises a functional signaling domain of DAP10 comprising an amino acid sequence of SEQ ID NO: 20. In some embodiments, the CAR signaling module comprises an intracellular signaling domain comprising a functional signaling domain of DAP12 comprising an amino acid sequence of SEQ ID NO: 21. In some embodiments, the CAR signaling module comprises an intracellular signaling domain comprising a functional signaling domain of 4-1 BB comprising an amino acid sequence of SEQ ID NO: 22. In some embodiments, the CAR signaling module comprises an intracellular signaling domain comprising a functional signaling domain of CD28 comprising an amino acid sequence of SEQ ID NO: 23. In some embodiments, the CAR signaling module comprises an intracellular signaling domain comprises a functional signaling domain of OX40 comprising an amino acid sequence of SEQ ID NO: 24.

In some embodiments, the CAR signaling module comprises a transmembrane domain of CD3 zeta, an intracellular signaling domain of CD3 zeta, and has an amino acid sequence of SEQ ID NOS: 29 or 30. In some embodiments, the CAR signaling module comprises a transmembrane domain of 2B4, a costimulatory signaling domain of 2B4, an intracellular signaling domain of CD3 zeta, and has an amino acid sequence of SEQ ID NOS 31 or 32. In some embodiments, the CAR signaling module comprises a transmembrane domain of DAP10, a costimulatory signaling domain of DAP10, an intracellular signaling domain of CD3 zeta, and has an amino acid sequence of SEQ ID NOS: 33 or 34. In some embodiments, a CAR signaling module comprises a transmembrane domain of DAP12, a costimulatory signaling domain of DAP12, an intracellular signaling domain of CD3 zeta, and has an amino acid sequence of SEQ ID NOS: 35 or 36. In some embodiments, the CAR signaling module comprises a transmembrane domain of CD28, a costimulatory signaling domain of CD28, an intracellular signaling domain of CD3 zeta, and has an amino acid sequence of SEQ ID NOS 37 or 38. In some embodiments, a CAR signaling module comprises a transmembrane domain of CD28, a first costimulatory signaling domain of CD28, a second costimulatory signaling domain of 4-1EE, an intracellular signaling domain of CD3 zeta, and has an amino acid sequence of SEQ ID NOS: 39 or 40. In some embodiments, a CAR signaling module comprises a transmembrane domain of 4-1BB, a costimulatory signaling domain of 4-1 BB, an intracellular signaling domain of CD3 zeta, and has an amino acid sequence of SEQ ID NO: 41. In some embodiments, a CAR signaling module comprises a transmembrane domain of CD8 alpha, a costimulatory signaling domain of CD28, an intracellular signaling domain of CD3 zeta, and has an amino acid sequence of SEQ ID NO: 42. In some embodiments, a CAR signaling module comprises a transmembrane domain of CD8 alpha, a costimulatory signaling domain of 4-1 BB, an intracellular signaling domain of CD3 zeta, and has an amino acid sequence of SEQ ID NO: 43. In some embodiments, a CAR signaling module comprises a transmembrane domain of CD8 alpha, a first costimulatory signaling domain of CD28, a second costimulatory signaling domain of 4-1 BB, an intracellular signaling domain of CD3 zeta, and has an amino acid sequence of SEQ ID NO: 44. In some embodiments, a CAR signaling module comprises a transmembrane domain of OX40, a costimulatory signaling domain of OX40, an intracellular signaling domain of CD3 zeta, and has an amino acid sequence of SEQ ID NO: 45. In some embodiments, a CAR signaling module comprises a transmembrane domain of OX40, a first costimulatory signaling domain of OX40, a second costimulatory signaling domain of 4-1BB, an intracellular signaling domain of CD3 zeta, and has an amino acid sequence of SEQ ID NO: 46. In some embodiments, a CAR signaling module comprises a transmembrane domain of CD8 alpha, a costimulatory signaling domain of OX40, an intracellular signaling domain of CD3 zeta, and has an amino acid sequence of SEQ ID NO: 47. In some embodiments, a CAR signaling module comprises a transmembrane domain of CD8 alpha, a first costimulatory domain of OX40, a second costimulatory signaling domain of 4-1 BB, an intracellular signaling domain of CD3 zeta, and has an amino acid sequence of SEQ ID NO: 48.

Also provided is a modular CAR system comprising: (A) a CAR signaling module, and (B) a potentiator module comprising (1) an NKp46 binding moiety and (2) a target cell binding moiety. The target cell may be any cell that is sought to be eliminated, for example a cancer cell. The target cell binding moiety can thus bind an antigen of interest on a target cells, for example target cell binding moiety can be specified to be a tumor antigen binding moiety. In some embodiments, the potentiator module comprises an Fc domain, or a portion an Fc domain capable of binding FcRn and/or optionally Fcγ receptors (e.g. CD16A). An Fc domain or fragment may be a human Fc domain or fragment with or without modifications (e.g. amino acid substitutions) to reduce or increase binding to human Fcγ receptors (e.g. CD16A). In some embodiments, the potentiator module comprises a CD16A-binding domain configured as an immunoglobulin binding domain that specifically binds to CD16A. In some embodiments, the potentiator module further comprises a binding domain that binds a human cytokine receptor present on NK cells (e.g., CD122). In some embodiments, the binding domain of the potentiator module binds a human cytokine receptor present on NK cells comprises an IL-2 moiety, or a variant thereof. In some embodiments, the NKp46 binding moiety of the potentiator module is capable of binding the extracellular NKp46 protein or fragment thereof comprised in the signaling module.

Also provided are nucleic acids encoding a CAR signaling module of the disclosure, as well as an expression vector comprising said nucleic acids.

The disclosure also provides cells made or modified (e.g., genetically modified) to express (e.g. at their surface) a CAR signaling module of the disclosure. In some embodiments, the cells are immune cells, preferably T cells or NK cells. In some embodiments, the cells can be allogeneic or autologous (e.g. with respect to the individual to be treated).

Also provided is a composition comprising cells expressing a CAR signaling module of the disclosure, and a pharmaceutically acceptable carrier.

Provided are cells expressing a CAR signaling module and CAR potentiator proteins for use as medicaments, for example for use in combination. In one embodiment, provided are cells expressing a CAR signaling module, for use in the treatment of disease (e.g. cancer), wherein the cells are for use in combination with a CAR potentiator protein. In one embodiment, provided is a CAR potentiator protein, for use in the treatment of disease (e.g. cancer), wherein the protein is for use in combination with cells expressing a CAR signaling module. The cells expressing a CAR signaling module and CAR potentiator module can be formulated separately. The cells expressing a CAR signaling module and CAR potentiator module can be for the simultaneous, sequential or separate administration to an individual. Also provided is a kit comprising a first composition comprising cells expressing a CAR signaling module according to the disclosure and a second composition comprising a CAR potentiator module. In another embodiment, provided is a kit comprising a composition comprising cells expressing a CAR signaling module according to the disclosure pre-complexed with CAR potentiator modules. The cells expressing CAR signaling module according to the invention pre-complexed with CAR potentiator module can advantageously be cryopreserved (e.g. frozen).

The disclosure also provides methods of making a cell expressing a CAR signaling module. Also provided are method of treating an individual comprising administering to said individual: (a) cells genetically modified to express a CAR signaling module, and (b) a protein (a CAR potentiator module) comprising a NKp46 binding moiety and target cell binding moiety (e.g. a tumor antigen binding moiety).

### BRIEF DESCRIPTION OF THE DRAWINGS

FIG. 1A represents an exemplary configuration of a CAR signaling module according to the disclosure. FIG. 1B represents an exemplary CAR potentiator module in a tetra-functional format (T5) that binds to an NK cell via the NKp46 domain of the CAR signaling module, CD16A via an Fc domain and to CD122/CD132 via an optional IL-2 receptor, and to an antigen on a tumor cell.

FIG. 2 represents the functioning of an exemplary modular CAR. The tetra-functional CAR potentiator module binds to a tumor antigen on the cancer cell and to CD16A, the CAR signaling module and CD122/CD132 on the NK cell. The NK cell is genetically modified to express the CAR signaling module. The genetically modified NK cell will then be able to lyse the cancer cell and have potent anti-tumor activity.

FIGS. 3A, 3B, 3C, 3D, 3E and 3F show different configurations of CAR potentiator modules that differ in the number of polypeptide chains, and in the configuration of the domains around an Fc domain dimer.

FIGS. 4A and 4B show % specific lysis induced by NK cells in a cytotoxicity assay at ET ratio 10:1. FIGS. 4C and 4D show % specific lysis induced by NK cells in a cytotoxicity assay at ET ratio 2:1. All of the NK cell engagers that retained the ability to bind both CD16 and NKp46 (in addition to CD20) displayed similarly high potency in terms of EC50 values in induction of NK cell cytotoxicity toward the tumor cells. The nature of the IL-2 polypeptide (either wild-type of the mutated IL2v) did not appear to differentially affect NK cell cytotoxicity, and furthermore the presence of IL2, whether as wild-type or IL2v, did not result in improved EC50 values in induction of cytotoxicity.

FIGS. 5 and 6 show the domain structure of different CAR signaling module constructs of the examples. FIG. 5 shows wild-type (WT) NK cells and CAR signaling modules SNK1, SNK2 and SNK3. WT NK cells have NKp46 (ECD and TM domain), which associates with Fc epsilon RI-gamma and CD3z cytoplasmic domains. SNK1 has an NKp46 ECD, a CD3z TM domain and a CD3z signaling domain. SNK2 has an NKp46 ECD, a 2B4 TM domain, a 2B4 costimulatory domain and a CD3z signaling domain. SNK3 has an NKp46 ECD, a 4-1EE TM domain, a 4-1BB costimulatory domain and a CD3z signaling domain.

FIG. 6 shows CAR signaling modules SNK4, SNK5 and SNK6. SNK4 has an NKp46 ECD, a CD28 TM domain, a CD28 costimulatory domain, a 4-1 BB costimulatory domain and a CD3z signaling domain. SNK5 has an NKp46 ECD, a CD8alpha TM domain, a CD28 costimulatory domain, a 4-1EE costimulatory domain and a CD3z signaling domain. SNK6 has an NKp46 ECD, an OX40 TM domain, an OX40 costimulatory domain and a CD3z signaling domain.

FIG. 7 shows levels of expression of SNK1, 4, and 6 NKp46-CAR signaling modules at the surface of KHYG-1 cells. Cells expressing the respective construct were analyzed by flow cytometry; and sorted for the expression of the NKp46-CAR at the cell surface. The level of expression of the NKp46-CAR at the cell surface was analyzed by flow cytometry as assessed by detection of NKp46. KHYG-1 WT are the parental cells (not transfected). Median fluorescence intensity values of NKp46 expression are indicated in the dot plots.

FIG. 8 shows cytotoxic activity of NK cells expressing NKp46-CAR signaling module SNK1. Cytotoxicity of CD20-CAR potentiator module (CD20-Potentiator) is shown against Daudi target cells with (SNK1) and without (WT) expression of NKp46-CAR signaling module SNK1 on KHYG-1 cells, used as effectors. Effector to target ratio of 20:1, 10:1 and 5:1 are shown. IC-Potentiator:Isotype control CAR potentiator module.

FIG. 9 shows cytotoxic activity of NK cells expressing NKp46-CAR signaling module SNK4. Cytotoxicity of CD20-CAR potentiator module (CD20-Potentiator) is shown against Daudi target cells with (SNK4) and without (WT) expression of NKp46-CAR signaling module SNK4 on KHYG-1 cells, used as effectors. Effector to target ratio of 20:1, 10:1 and 5:1 are shown. IC-Potentiator:Isotype control CAR potentiator module.

FIG. 10 shows cytotoxic activity of NK cells expressing NKp46-CAR signaling module SNK6. Cytotoxicity of CD20-CAR potentiator module (CD20-Potentiator) is shown against Daudi target cells with (SNK6) and without (WT) expression of NKp46-CAR signaling module SNK6 on KHYG-1 cells, used as effectors. Effector to target ratio of 20:1, 10:1 and 5:1 are shown. IC-Potentiator:Isotype control CAR potentiator module.

FIG. 11A shows a schematic representation of Jurkat-SNK2 CAR-NKp46 and Jurkat-SNK4 CAR-NKp46 constructs as expressed at the surface of engineered Jurkat T cells, including the NKp46 extracellular domain (NKp46-ECD), the transmembrane domain (Tm) and signaling module domains.

FIG. 11B shows results of flow cytometry analysis of the transfected and control Jurkat cells after staining with anti-NKp46 antibody (9E2-PE). Fluorescence intensity (FI) of NKp46 staining is shown on the x-axis and side scatter (SSC) is shown on the y-axis. Percent of NKp46-positive cells are indicated in the graph. The figure shows non-transfected wild-type (WT) Jurkat cells do not express NKp46 ECD at their cell surface, while 90.2% of Jurkat SNK2 cells and 88.1% of Jurkat SNK4 cells expressed NKp46 ECD at their cell surface.

FIGS. 12A and 12B show levels of CD69 and CD25 expression at the cell surface of Jurkat SNK2 & SNK4, respectively, induced by CD20-NKCE CAR potentiator modules. The x-axis shows concentration of test molecules and the y-axis shows median fluorescence intensity (MedFI) on the right hand panels and on the left hand panels percent positive cells expressing CD69 and CD25.

FIG. 13 shows that CD20-NKCE CAR potentiator module induces target dependent IL-2 secretion by Jurkat SNK2 & SNK4 cells. The x-axis shows concentration of test molecules and the y-axis shows IL-2 concentrations (pg/mL).

FIG. 14 shows a schematic representation of the CD3z and FceRIg constructs used to transfect NK cells (KHYG cell line) in order to complement regular NKp46 cell surface expression in the NK cells.

FIG. 15, top panel, shows expression of EGFP and BFP2 associated to CD3z and FceRIg constructs in KHYG-1 sub-clones monitored by flow cytometry. FIG. 15, bottom panel, shows expression of NKp46 at the cell surface of KHYG-1 sub-clones expressing CAR-NKP46, CD3z and FceRIg constructs. Values of median fluorescence intensity (MedFI) are indicated.

FIGS. 16, 17, 18 and 19 show cytotoxicity (% specific lysis) mediated by parental KHYG-1 NK cells and KHYG-1 sub-clones expressing CAR-NKp46, CD3z, or FceRIg constructs, in the presence or absence of different concentrations of a CAR potentiator module that binds to the tumor antigen CD20 (CD20-NKCE) or a comparator protein lacking the tumor-binding moiety (IC-NKCE). FIG. 16 and FIG. 17 respectively show a first and a second experiment with NK cells at an E/T ratio of 5:1. FIG. 18 and FIG. 19 respectively show a first and a second experiment with NK cells at an E/T ratio of 1:1.

### DETAILED DESCRIPTION

### Definitions

As used in the specification, “a” or “an” may mean one or more. As used in the claim(s), when used in conjunction with the word “comprising”, the words “a” or “an” may mean one or more than one.

Where “comprising” is used, this can optionally be replaced by “consisting essentially of”, or optionally by “consisting of”.

As used herein, the term “antigen binding domain” or “ABD” refers to a domain comprising a three-dimensional structure capable of immunospecifically binding to an epitope. Thus, in one embodiment, said domain can comprise a hypervariable region, optionally a VH and/or VL domain of an antibody chain, optionally at least a VH domain. In another embodiment, the binding domain may comprise at least one complementarity determining region (CDR) of an antibody chain. In another embodiment, the binding domain may comprise a polypeptide domain from a non-immunoglobulin scaffold.

The term “antibody” herein is used in the broadest sense and specifically includes full-length monoclonal antibodies, polyclonal antibodies, multispecific antibodies (e.g., bispecific antibodies), and antibody fragments and derivatives, so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988). An “antibody fragment” comprises a portion of a full-length antibody, e.g. antigen-binding or variable regions thereof. Examples of antibody fragments include Fab, Fab′, F(ab)2, F(ab′)2, F(ab)3, Fv (typically the VL and VH domains of a single arm of an antibody), single-chain Fv (scFv), dsFv, Fd fragments (typically the VH and CH1 domain), and dAb (typically a VH domain) fragments; VH, VL, VhH, and V-NAR domains; minibodies, diabodies, triabodies, tetrabodies, and kappa bodies (see, e.g., Ill et al., Protein Eng 1997; 10: 949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23, 1126-1136; WO2005040219, and published U.S. Patent Applications 20050238646 and 20020161201.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

By “constant region” as defined herein is meant an antibody-derived constant region that is encoded by one of the light or heavy chain immunoglobulin constant region genes.

By “constant light chain” or “light chain constant region” or “CL” as used herein is meant the region of an antibody encoded by the kappa (Cκ) or lambda (Cλ) light chains. The constant light chain typically comprises a single domain, and as defined herein refers to positions 108-214 of Cκ, or Cλ, wherein numbering is according to the EU index (Kabat et al., 1991, Sequences of Proteins of Immunological Interest, 5th Ed., United States Public Health Service, National Institutes of Health, Bethesda).

By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

By “Fab” or “Fab region” as used herein is meant a unit that comprises the VH, CH1, VL, and CL immunoglobulin domains. The term Fab includes a unit that comprises a VH-CH1 moiety that associates with a VL-CL moiety, as well as crossover Fab structures in which there is crossing over or interchange between light- and heavy-chain domains. For example a Fab may have a VH-CL unit that associates with a VL-CH1 unit. Fab may refer to this region in isolation, or this region in the context of a protein, multispecific protein or ABD, or any other embodiments as outlined herein.

By “single-chain Fv” or “scFv” as used herein are meant antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994).

By “Fv” or “Fv fragment” or “Fv region” as used herein is meant a polypeptide that comprises the VL and VH domains of a single antibody.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cγ3 (CH3) and optionally the hinge between Cγ1 and Cγ2. Although the boundaries of the Fc region may vary, the human IgG heavy chain Fc region is usually defined to comprise residues C226, P230 or A231 to its carboxyl-terminus, wherein the numbering is according to the EU index. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides herein include but are not limited to antibodies, Fc fusions and Fc fragments. Also, Fc regions according can include variants containing at least one modification that alters (enhances or diminishes) an Fc associated effector function. Also, Fc regions according can include chimeric Fc regions comprising different portions or domains of different Fc regions, e.g., derived from antibodies of different isotype or species.

By “variable region” as used herein is meant the region of an antibody that comprises one or more Ig domains substantially encoded by any of the VL (including Vκ (Vκ) and Vλ) and/or VH genes that make up the light chain (including κ and λ) and heavy chain immunoglobulin genetic loci respectively. A light or heavy chain variable region (VL or VH) consists of a “framework” or “FR” region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)), and as in Chothia. The framework regions of an antibody, that is the combined framework regions of the constituent light and heavy chains, serves to position and align the CDRs, which are primarily responsible for binding to an antigen.

The term “specifically binds to” means that an antibody or polypeptide can bind preferably in a competitive binding assay to the binding partner, e.g. NKp46, as assessed using either recombinant forms of the proteins, epitopes therein, or native proteins present on the surface of isolated target cells. Competitive binding assays and other methods for determining specific binding are further described below and are well known in the art.

When an antibody or polypeptide is said to “compete with” a particular multispecific protein, a CAR potentiator module, or a particular monoclonal antibody (e.g. NKp46-1, -2, -4, -6 or -9 in the context of an anti-NKp46 mono-specific antibody or a multi-specific protein), it means that the antibody or polypeptide competes with the particular multispecific protein or monoclonal antibody in a binding assay using either recombinant target (e.g. NKp46) molecules or surface expressed target (e.g. NKp46) molecules. For example, if a test antibody reduces the binding of NKp46-1, -2, -4, -6 or -9 to a NKp46 polypeptide or NKp46-expressing cell in a binding assay, the antibody is said to “compete” respectively with NKp46-1, -2, -4, -6 or -9.

The term “affinity”, as used herein, means the strength of the binding of an antibody or protein to an epitope. The affinity of an antibody is given by the dissociation constant KD, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant KA is defined by 1/KD. Preferred methods for determining the affinity of proteins can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of proteins is the use of surface plasmon resonance (SPR) screening (such as by analysis with a BIAcore™ SPR analytical device).

Within the context herein a “determinant” designates a site of interaction or binding on a polypeptide.

The term “epitope” refers to an antigenic determinant, and is the area or region on an antigen to which an antibody or protein binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’. Epitopes may be identified by different methods known in the art including but not limited to alanine scanning, phage display, X-ray crystallography, array-based oligo-peptide scanning or pepscan analysis, site-directed mutagenesis, high throughput mutagenesis mapping, H/D-Ex Mass Spectroscopy, homology modeling, docking, hydrogen-deuterium exchange, among others. (See e.g., Tong et al., Methods and Protocols for prediction of immunogenic epitopes”, Briefings in Bioinformatics 8(2):96-108; Gershoni, Jonathan M; Roitburd-Berman, Anna; Siman-Tov, Dror D; Tarnovitski Freund, Natalia; Weiss, Yael (2007). “Epitope Mapping”. BioDrugs 21 (3): 145-56; and Flanagan, Nina (May 15, 2011); “Mapping Epitopes with H/D-Ex Mass Spec: ExSAR Expands Repertoire of Technology Platform Beyond Protein Characterization”, Genetic Engineering & Biotechnology News 31 (10).

“Valent” or “valency” denotes the presence of a determined number of antigen-binding moieties in the antigen-binding protein. A natural IgG has two antigen-binding moieties and is bivalent. A molecule having one binding moiety for a particular antigen is monovalent for that antigen. By “amino acid modification” herein is meant an amino acid substitution, insertion, and/or deletion in a polypeptide sequence. An example of amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y50W refers to a variant of a parent polypeptide, in which the tyrosine at position 50 is replaced with tryptophan. Amino acid substitutions are indicated by listing the residue present in wild-type protein/position of residue/residue present in mutant protein. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of a polypeptide will exhibit 98%, 98%, or 99% homogeneity for polypeptides in the context of all present peptide species in the composition or at least with respect to substantially active peptide species in the context of proposed use.

“NK cells” refers to a sub-population of lymphocytes that is involved in non-conventional immunity. NK cells can be identified by virtue of certain characteristics and biological properties, such as the expression of specific surface antigens including CD56 and/or NKp46 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell KS and Colonna M). Humana Press. pp. 219-238 (2000).

The term “subject” or “individual” or “patient” are used interchangeably and may encompass a human or a non-human mammal, rodent or non-rodent. The term includes, but is not limited to, mammals, e.g., humans including man, woman and child, other primates (monkey), pigs, rodents such as mice and rats, rabbits, guinea pigs, hamsters, cows, horses, cats, dogs, sheep and goats.

### Modular Chimeric Activating Receptor Systems

Disclosed herein is a modular chimeric activating receptor system, the system comprising at least:
(A) a signaling module comprising at least (i) an extracellular domain; (ii) a transmembrane domain; and (iii) an intracellular signaling domain; and
(B) a potentiator module comprising at least (1) a binding moiety capable of interacting with (e.g. binding) an extracellular domain of the signaling module of (A) and (2) a target antigen (e.g. tumor antigen) binding moiety. In one aspect, the signaling module (A) is embodied as a cell expressing (e.g. made to express) the signaling module. A binding moiety of a potentiator module can also be referred to as an antigen binding domain, e.g. any antigen binding domain further described herein.

The term chimeric activating receptor system can is some aspects be described as a system (e.g., a set of at least two polypeptides, a set of at least two polypeptides wherein the potentiator module polypeptide is provided as a cell (e.g. NK cell) expressing the potentiator). When the potentiator module is expressed at the surface of an immune effector cell, it provides the immune effector cell with a specificity for the soluble potentiator module protein.

In some embodiments a CAR signaling module comprises at least an extracellular ECD binding domain, a transmembrane domain and a cytoplasmic signaling domain (also called intracellular signaling domain) comprising a functional signaling domain derived from a stimulatory molecule and/or costimulatory molecule as defined in the specification.

### A) CAR Signaling Module

Disclosed herein are chimeric activating receptor signaling modules that comprise at least (i) an extracellular domain; (ii) a transmembrane domain; and (iii) an intracellular signaling domain.

### (i) CAR Intracellular Domains

An intracellular component of a CAR signaling module includes one or more intracellular signaling domains. In some embodiments, the intracellular signaling domain generates (e.g. is capable of generating) a signal that promotes an immune effector function of the cell (e.g. activates an NK cell) that expresses the CAR signaling module. Examples of immune effector functions include cytolytic activity (e.g. as assessed by any suitable known marker of cytotoxicity for NK cells) and cytokine production.

A signaling domain can be referred to as a functional portion of a protein which acts by transmitting information within the cell to regulate cellular activity via defined signaling pathways by generating second messengers or functioning as effectors by responding to such messengers. A signaling domain can therefore be characterized as being involved in mediating stimulation. Stimulation refers to a primary response induced by binding of a stimulatory molecule (e.g., a CAR) with its cognate ligand, thereby mediating a signal transduction event, such as signal transduction via appropriate signaling domains of the CAR signaling module. Stimulation can mediate altered expression of certain molecules. A stimulatory molecule can refer to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In some embodiments, the signal is a primary signal that is initiated by, for instance, binding of a CAR to a CAR potentiator module that is bound (e.g. upon administration to an individual) to a antigen expressed by a tumor, which leads to mediation of an immune cell response, including proliferation, activation and/or differentiation.

An intracellular signaling domain can include the entire intracellular portion, or the entire native intracellular signaling domain, of the molecule from which it is derived, or a functional fragment or derivative thereof. In some embodiments, an intracellular signaling domain can include a primary intracellular signaling domain. In some embodiments, primary intracellular signaling domains include those derived from the molecules responsible for primary stimulation, or antigen dependent stimulation. In some embodiments, the intracellular signaling domain can further include a costimulatory intracellular domain.

A primary intracellular signaling domain can include a signaling motif which is known as an immunoreceptor tyrosine-based activation motif or ITAM. An ITAM motif is a four amino acids motifs composed of a tyrosine Y separated from a Leucine L or an isoleucine I by two other amino acids, leading to the following signature: YxxL/I. Examples of ITAM containing primary cytoplasmic signaling sequences include those derived from CD3 zeta, common FcR gamma (FCER1G), Fc gamma RIIa, FcR beta (Fc epsilon R1b), CD3 gamma, CD3 delta, CD3 epsilon, CD79a, CD79b, DAP10, and DAP12, or a combination thereof.

In some embodiments, a CD3 zeta (CD247) stimulatory domain can include amino acid residues from the cytoplasmic domain of the T cell receptor zeta chain, or functional derivatives thereof, that are sufficient to functionally transmit an initial signal necessary for cell activation. In some embodiments, a CD3 zeta stimulatory domain can include human CD3 zeta stimulatory domain or functional orthologs thereof. In some embodiments, a human CD3 zeta stimulatory domain includes SEQ ID NO: 18, or an amino acid sequence at least 90%, 95%, 98% or 99% identical thereto, or a contiguous sequence of at least 20, 30 or 40 amino acid residues thereof. In some embodiments, in the case of an intracellular signaling domain that is derived from a CD3 zeta molecule, the intracellular signaling domain retains sufficient CD3 zeta structure such that it can generate a signal under appropriate conditions.

In some embodiments, the intracellular signaling domain can include a costimulatory intracellular domain. In some embodiments, costimulatory intracellular signaling domains include those derived from molecules responsible for costimulatory signals, or antigen independent stimulation. In some embodiments, a costimulatory intracellular signaling domain can be the intracellular portion of a costimulatory molecule. A costimulatory molecule refers to a cognate binding partner on an immune cell that specifically binds with a costimulatory ligand, thereby mediating a costimulatory response by the immune cell, such as proliferation. Costimulatory molecules include cell surface molecules other than antigen receptors or their ligands that contribute to an efficient immune response. A costimulatory molecule can be represented in the following protein families: TNF receptor proteins, immunoglobulin-like proteins, cytokine receptors, integrins, signaling lymphocytic activation molecules (SLAM proteins), and activating NK cell receptors. Examples of such molecules include: an MHC class I molecule, B and T cell lymphocyte attenuator (BTLA, CD272), a Toll ligand receptor, CD27, CD28, 4-1 BB (CD137), OX40, GITR, CD30, CD40, ICOS (CD278), BAFFR, HVEM (LIGHTR), ICAM-1, lymphocyte function-associated antigen-1 (LFA-1; CD11a/CD18), CD2, CDS, CD7, CD287, LIGHT, NKG2C, NKG2D, SLAMF7, NKp80 (KLRF1), NKp30, NKp44, NKp46, CD160 (BY55), B7-H3 (CD276), CD19, CD4, CD8 alpha, CD8 beta, IL2R beta, IL2R gamma, IL7R alpha, ITGA4, VLA1, CD49a, IA4, CD49d, ITGA6, VLA-6, CD49f, ITGAD, CD11d, ITGAE, CD103, ITGAL, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, and the like, or a combination thereof.

In some embodiments, a costimulatory intracellular signaling domain includes 4-1BB (CD137, TNFRSF9). 4-1BB refers to a member of the tumor necrosis factor receptor (TNFR) superfamily. In some embodiments, a 4-1BB costimulatory domain includes a human 4-1 BB costimulatory domain or a functional ortholog thereof. In some embodiments, a human 4-1 BB costimulatory domain comprises the amino acid sequence of SEQ ID NO: 22 or an amino acid sequence at least 90%, 95%, 98% or 99% identical thereto, or a contiguous sequence of at least 20, 30 or 40 amino acid residues thereof.

In some embodiments, a costimulatory intracellular signaling domain includes 2B4 (CD244). 2B4 belongs to the signaling lymphocytic activation molecule (SLAM) family and binds to CD48. In some embodiments, a 2B4 costimulatory domain includes a human 2B4 costimulatory domain or a functional ortholog thereof. In some embodiments, a human 2B4 costimulatory domain comprises the amino acid sequence of SEQ ID NO: 19, or an amino acid sequence at least 90%, 95%, 98% or 99% identical thereto, or a contiguous sequence of at least 20, 30 or 40 amino acid residues thereof.

In some embodiments, a costimulatory intracellular signaling domain includes DAP10. DAP10 (DNAX-activating protein 10) is a transmembrane adaptor protein that binds to receptor for advanced glycation end-products (RAGE). In one embodiment, a DAP10 costimulatory domain includes a human DAP10 costimulatory domain or a functional ortholog thereof. In some embodiments, a human DAP10 costimulatory domain comprises the amino acid sequence of SEQ ID NO: 20, or an amino acid sequence at least 90%, 95%, 98% or 99% identical thereto, or a contiguous sequence of at least 20, 30 or 40 amino acid residues thereof.

In some embodiments, a costimulatory intracellular domain includes DAP12. DAP12 (DNAX-activating protein 12) is a key signal transduction receptor. In one embodiment, a DAP12 costimulatory domain includes a human DAP12 costimulatory domain or a functional ortholog thereof. In some embodiments, a human DAP12 costimulatory domain comprises the amino acid sequence of SEQ ID NO: 21, or an amino acid sequence at least 90%, 95%, 98% or 99% identical thereto, or a contiguous sequence of at least 20, 30 or 40 amino acid residues thereof.

In some embodiments, a costimulatory intracellular domain includes CD28. CD28 is a homodimer of disulfide-linked chains that is involved in the interaction of T lymphocytes with antigen-presenting cells. In one embodiment, a CD28 costimulatory domain includes a human CD28 costimulatory domain or a functional ortholog thereof. In some embodiments, a CD28 costimulatory domain comprises the amino acid sequence of SEQ ID NO: 23, or an amino acid sequence at least 90%, 95%, 98% or 99% identical thereto, or a contiguous sequence of at least 20, 30 or 40 amino acid residues thereof.

In some embodiments, a costimulatory intracellular domain includes OX40. OX40 (CD134 or TNFRSF4) belongs to the TNFR/TNF superfamily and is expressed on activated CD4 and CD8 T cells as well as a number of other lymphoid and non-lymphoid cells. In one embodiment, a OX40 costimulatory domain includes a human OX40 costimulatory domain includes a human OX40 costimulatory domain or a functional ortholog thereof. In some embodiments, a OX40 costimulatory domain comprises the amino acid sequence of SEQ ID NO: 24, or an amino acid sequence at least 90%, 95%, 98% or 99% identical thereto, or a contiguous sequence of at least 20, 30 or 40 amino acid residues thereof.

### (ii) CAR Transmembrane Domains

A CAR signaling module can be designed to include a transmembrane domain that links the extracellular component to the intracellular component of the CAR signaling module. A transmembrane domain can anchor a CAR signaling module to a cell membrane. A transmembrane domain can include one or more additional amino acids adjacent to the transmembrane region, e.g., one or more amino acids associated with the extracellular region of the protein from which the transmembrane was derived (e.g., 1, 2, 3, 4, 5, 6, 7, 8, 9, 10, 11, 12, 13, 14, 15 amino acids, or more of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15 amino acids, or more of the intracellular region). In some embodiments, the transmembrane domain may be from the same protein that the signaling domain, costimulatory domain, or the hinge domain is derived from. In some embodiments, the transmembrane domain is not derived from the same protein that any other domain of the CAR is derived from. In some embodiments, the transmembrane domain can be selected or modified by amino acid substitution to avoid binding of or to minimize interactions with other domains in the CAR.

In some embodiments, a transmembrane domain has a three-dimensional structure that is thermodynamically stable in a cell membrane, and generally ranges in length from 15 to 30 amino acids. The structure of a transmembrane domain can include an alpha helix, a beta barrel, a beta sheet, a beta helix, or any combination thereof.

The transmembrane domain may be derived from any membrane-bound or transmembrane protein. In some embodiments, the transmembrane domain is capable of signaling to the intracellular domain(s) whenever the CAR has bound to a target. In some embodiments, a transmembrane domain may include at least the transmembrane region(s) of: the alpha, beta, or zeta chain of the T-cell receptor; CD28; CD27; CD3 zeta; CD3 epsilon; CD45; CD4; CD5; CD8; CD9; CD16; CD22; CD33; CD37; CD64; CD80; CD86; CD134; CD137; and/or CD154. In some embodiments, a transmembrane domain may include at least the transmembrane region(s) of: KIRDS2; OX40; CD2; LFA-1; ICOS; 4-1 BB; GITR; CD40; BAFFR; HVEM; SLAMF7; NKp80; NKp44; NKp30; NKp46; CD8 alpha; CD160; CD19; IL2R beta; IL2R gamma; IL7Ra; ITGA1; VLA1; CD49a; ITGA4; IA4; CD49D; ITGA6; VLA-6; CD49f; ITGAD; CDI Id; ITGAE; CD103; ITGAL; CDI Ia; ITGAM; CDI Ib; ITGAX; CDI Ic; ITGB1; CD29; ITGB2; CD18; ITGB7; TNFR2; DAP10; DAP12; 2B4 DNAM1; SLAMF4; CD84; CD96; CEACAM1; CRT AM; Ly9; CD160; PSGL1; CD100; SLAMF6 (NTB-A, Ly108); SLAM; BLAME; SELPLG; LTBR; PAG/Cbp; NKG2D; NKG2C; or a combination thereof.

In some embodiments, a transmembrane domain of a CAR signaling module includes a transmembrane domain from CD3 zeta (CD3z). In some embodiments, a CD3 zeta transmembrane domain comprises an amino acid sequence of SEQ ID NO: 5, or an amino acid sequence at least 90%, 95%, 98% or 99% identical thereto, or a contiguous sequence of at least 5, 10 or 20 amino acid residues thereof. In other embodiments, a CD3 zeta transmembrane domain comprises an amino acid sequence of SEQ ID NO: 6, or an amino acid sequence at least 90%, 95%, 98% or 99% identical thereto, or a contiguous sequence of at least 5, 10 or 20 amino acid residues thereof.

In some embodiments, a transmembrane domain of a CAR signaling module includes a transmembrane domain from 2B4. In some embodiments, a 2B4 transmembrane domain comprises an amino acid sequence of SEQ ID NO: 7, or an amino acid sequence at least 90%, 95%, 98% or 99% identical thereto, or a contiguous sequence of at least 5, 10 or 20 amino acid residues thereof. In other embodiments, a 2B4 transmembrane domain comprises an amino acid sequence of SEQ ID NO: 8, or an amino acid sequence at least 90%, 95%, 98% or 99% identical thereto, or a contiguous sequence of at least 5, 10 or 20 amino acid residues thereof.

In some embodiments, a transmembrane domain of a CAR signaling module includes a transmembrane domain from DAP10. In some embodiments, a DAP10 transmembrane domain comprises an amino acid sequence of SEQ ID NO: 9, or an amino acid sequence at least 90%, 95%, 98% or 99% identical thereto, or a contiguous sequence of at least 5, 10 or 20 amino acid residues thereof. In other embodiments, a DAP10 transmembrane domain comprises an amino acid sequence of SEQ ID NO: 10, or an amino acid sequence at least 90%, 95%, 98% or 99% identical thereto, or a contiguous sequence of at least 5, 10 or 20 amino acid residues thereof.

In some embodiments, a transmembrane domain of a CAR signaling module includes a transmembrane domain from DAP12. In some embodiments, a DAP12 transmembrane domain comprises an amino acid sequence of SEQ ID NO: 11, or an amino acid sequence at least 90%, 95%, 98% or 99% identical thereto, or a contiguous sequence of at least 5, 10 or 20 amino acid residues thereof. In other embodiments, a DAP12 transmembrane domain comprises an amino acid sequence of SEQ ID NO: 12, or an amino acid sequence at least 90%, 95%, 98% or 99% identical thereto, or a contiguous sequence of at least 5, 10 or 20 amino acid residues thereof.

In some embodiments, a transmembrane domain of a CAR signaling module includes a transmembrane domain from 4-1BB. In some embodiments, a 4-1BB transmembrane domain comprises an amino acid sequence of SEQ ID NO: 13, or an amino acid sequence at least 90%, 95%, 98% or 99% identical thereto, or a contiguous sequence of at least 5, 10 or 20 amino acid residues thereof.

In some embodiments, a transmembrane domain of a CAR signaling module includes a transmembrane domain from CD28. In some embodiments, a CD28 transmembrane domain comprises an amino acid sequence of SEQ ID NO: 14, or an amino acid sequence at least 90%, 95%, 98% or 99% identical thereto, or a contiguous sequence of at least 5, 10 or 20 amino acid residues thereof. In other embodiments, a CD28 transmembrane domain comprises an amino acid sequence of SEQ ID NO: 15, or an amino acid sequence at least 90%, 95%, 98% or 99% identical thereto, or a contiguous sequence of at least 5, 10 or 20 amino acid residues thereof.

In some embodiments, a transmembrane domain of a CAR signaling module includes a transmembrane domain from CD8 alpha. In some embodiments, a CD8 alpha transmembrane domain comprises an amino acid sequence of SEQ ID NO: 16, or an amino acid sequence at least 90%, 95%, 98% or 99% identical thereto, or a contiguous sequence of at least 5, 10 or 20 amino acid residues thereof.

In some embodiments, a transmembrane domain of a CAR signaling module includes a transmembrane domain from OX40. In some embodiments, a OX40 transmembrane domain comprises an amino acid sequence of SEQ ID NO: 17, or an amino acid sequence at least 90%, 95%, 98% or 99% identical thereto, or a contiguous sequence of at least 5, 10 or 20 amino acid residues thereof.

(iii) CAR Spacer

As used herein, a linker within a CAR signaling module can be any portion of a CAR molecule that serves to connect two components or domains of the molecule. In some embodiments, linkers can provide flexibility for a CAR or module/portion of a CAR. Spacer regions are a type of linker region that are used to create appropriate distances and/or flexibility from other linked components. In some embodiments, the length of a spacer region can be customized for individual cellular markers on tumor cells to optimize tumor cells recognition and destruction. The spacer can be of a length that provides for increased responsiveness of the CAR expressing cell following binding to a CAR potentiator module bound or anchored to an antigen on a target cell, as compared to in the absence of the spacer. Spacer regions can also allow for high expression levels in CAR modified cells. In some embodiments, an extracellular spacer region of a CAR is located between a transmembrane domain and the extracellular binding domain.

Exemplary spacers include those having 10 to 250 amino acids, 10 to 200 amino acids, 10 to 150 amino acids, 10 to 100 amino acids, 10 to 50 amino acids, or 10 to 25 amino acids. In some embodiments, a spacer region is 12 amino acids, 20 amino acids, 21 amino acids, 26 amino acids, 27 amino acids, 45 amino acids, or 50 amino acids. In some embodiments, a longer spacer is greater than 119 amino acids, an intermediate spacer is 13-119 amino acids, and a short spacer is 10-12 amino acids.

Junction amino acids can be a linker which can be used to connect the sequences of CAR domains when the distance provided by a spacer is not needed and/or wanted. In some embodiments, junction amino acids are short amino acid sequences that can be used to connect intracellular signaling domains. In some embodiments, junction amino acids are 9 amino acids or less.

Junction amino acids can be a short oligo- or protein linker, preferably between 2 and 9 amino acids (e.g., 2, 3, 4, 5, 6, 7, 8, or 9 amino acids) in length to form the linker. In some embodiments, a glycine-serine doublet can be used as a suitable junction amino acid linker. In some embodiments, a single amino acid, e.g., an alanine, a glycine, can be used as a suitable junction amino acid.

In some embodiments, a short oligo- or polypeptide linker, between 2 and 9 amino acids in length may link the transmembrane domain and the intracellular component of the CAR. A glycine-serine doublet provides a particularly suitable linker. In some embodiments, a linker can include SEQ ID NOs: 25-28.

### (iv) CAR Extracellular NKp46 Domain

In some embodiments, a CAR signaling module can include an extracellular domain, preferably an extracellular domain of a protein expressed at the surface of an NK cell.

In some embodiments, a NK cell protein is a natural cytotoxicity receptor (NCR). In particular embodiment, a NK cell marker is selected from NKp44, NKp46 and NKp30. In some embodiments, a NK cell cellular marker is NKp46. NKp46 is a 46 kDa type I transmembrane glycoprotein with extracellular (Ig) domains, a transmembrane domain containing a positively charged amino acid residue, and a short cytoplasmic tail. NKp46 is encoded by the Ncr1 gene or by a cDNA prepared from such a gene. Any naturally occurring isoform, allele, ortholog or variant is encompassed by the term NKp46 polypeptide (e.g., an NKp46 polypeptide 80%, 90%, 95%, 98% or 99% identical to the amino acid sequence of SEQ ID NO: 1, or a contiguous sequence of at least 20, 30, 50, 100 or 200 amino acid residues thereof). The 304 amino acid residue sequence of human NKp46 (isoform a) is of SEQ ID NO: 1, as reproduced below:

(SEQ ID NO: 1)
MSSTLPALLC VGLCLSQRIS AQQQTLPKPF IWAEPHFMVP KEKQVTICCQ GNYGAVEYQL
HFEGSLFAVD RPKPPERINK VKFYIPDMNS RMAGQYSCIY RVGELWSEPS NLLDLVVTEM
YDTPTLSVHP GPEVISGEKV TFYCRLDTAT SMFLLLKEGR SSHVQRGYGK VQAEFPLGPV
TTAHRGTYRC FGSYNNHAWS FPSEPVKLLV TGDIENTSLA PEDPTFPADT WGTYLLTTET
GLQKDHALWD HTAQNLLRMG LAFLVLVALV WFLVEDWLSR KRTRERASRA STWEGRRRLN
TQTL.

The amino acid sequence of SEQ ID NO: 1 corresponds to NKBI accession number NP_004820, the disclosure of which is incorporated by reference. The human NKp46 mRNA sequence is described in NCBI accession number NM_004829, also incorporated herein by reference.

The NKp46 ECD can for example be characterized as comprising the D1 domain of NKp46, the D2 domain of NKp46 and/or a region spanning the D1 and D2 domains (at the border of the D1 and D2 domains) referred to as the D1/D2 junction.

In some embodiments, a CAR signaling module comprises a fragment of NKp46. In some embodiments, a CAR signaling module comprises the extracellular binding domain of NKp46, or a fragment thereof, e.g. an NKp46 polypeptide fragment having an amino acid sequence 80%, 90%, 95%, 98% or 99% identical to the amino acid sequence of SEQ ID NOS: 2 or 3, or a contiguous sequence of at least 20, 30, 50, 100 or 200 amino acid residues thereof. The extracellular binding domain of NKp46 is a polypeptide of 258 amino acid residues having the amino acid sequence of SEQ ID NO: 2, as reproduced below (including the peptide leader).

(SEQ ID NO: 2)
MSSTLPALLC VGLCLSQRIS AQQQTLPKPF IWAEPHFMVP KEKQVTICCQ GNYGAVEYQL
HFEGSLFAVD RPKPPERINK VKFYIPDMNS RMAGQYSCIY RVGELWSEPS NLLDLVVTEM
YDTPTLSVHP GPEVISGEKV TFYCRLDTAT SMFLLLKEGR SSHVQRGYGK VQAEFPLGPV
TTAHRGTYRC FGSYNNHAWS FPSEPVKLLV TGDIENTSLA PEDPTFPADT WGTYLLTTET
GLQKDHALWD HTAQNLLR

The mature extracellular domain of NKp46 (without the peptide leader) has an amino acid sequence of SEQ ID NO: 3, as reproduced below.

(SEQ ID NO: 3)
QQQTLPKPFI WAEPHFMVPK EKQVTICCQG NYGAVEYQLH FEGSLFAVDR PKPPERINKV
KFYIPDMNSR MAGQYSCIYR VGELWSEPSN LLDLVVTEMY DTPTLSVHPG PEVISGEKVT
FYCRLDTATS MFLLLKEGRS SHVQRGYGKV QAEFPLGPVT TAHRGTYRCF
GSYNNHAWSF PSEPVKLLVT GDIENTSLAP EDPTFPADTW GTYLLTTETG
LQKDHALWDH TAQNLLR

In some embodiments, a CAR signaling module comprises an extracellular NKp46 domain having an amino acid sequence of SEQ ID NO: 2 or SEQ ID NO: 3 a variant thereof having at least 85%, 90%, 95%, 98%, 99% of identity with the amino acid sequences of SEQ ID NO: 2 or SEQ ID NO: 3. For example, a variant of an extracellular NKp46 domain that can be comprised in a CAR signaling module can have an amino acid sequence of SEQ ID NO: 4.

### (v) Format of CARs Signaling Modules

In some embodiments, a CAR signaling module can be constructed according to the following domains arrangement:

NKp46 ECD-TM-CO1-CO2-Signaling module
Wherein:
TM is a transmembrane domain as described herein,
CO1 is a first costimulatory domain as described herein, and can be present or absent,
CO2 is a second costimulatory domain as described herein, and can be present or absent.

In some embodiments, a CAR signaling module comprises an NKp46 extracellular domain (ECD) and a transmembrane and signaling module domains from CD3 zeta, according to the following domains arrangement:
NKp46 ECD-CD3 zeta TM-CD3 zeta signaling module

In a particular embodiment, a CAR signaling module comprises the following amino acid sequence:

(SEQ ID NO: 29)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRLCYLLDGILFIYGVILTALFLRVKFSRSADAPAYQQGQNQLYNELNLGRRE
EYDVLDKRRGRDPEMGGKPQRRKNPQEGLYNELQKDKMAEAYSEIGMKGERRRGKGHD
GLYQGLSTATKDTYDALHMQALPPR

In an alternative embodiment, a CAR signaling module comprises the following amino acid sequence:

(SEQ ID NO: 30)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLDPKLCYLLDGILFIYGVILTALFLRVKFSRSADAPAYQQGQNQLYNELNLGR
REEYDVLDKRRGRDPEMGGKPQRRKNPQEGLYNELQKDKMAEAYSEIGMKGERRRGKGH
DGLYQGLSTATKDTYDALHMQALPPR

In some embodiments, a CAR signaling module comprises an extracellular NKp46 domain, a transmembrane domain and costimulatory domain from 2B4, and a signaling module domain from CD3 zeta, according to the following domains arrangement:
NKp46 ECD-2B4 TM-2B4 CO1-CD3 zeta signaling module

In a particular embodiment, a CAR signaling module comprises the following amino acid sequence:

(SEQ ID NO: 31)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRFLVIIVILSALFLGTLACFCVWRRKRKEKQSETSPKEFLTIYEDVKDLKTRR
NHEQEQTFPGGGSTIYSMIQSQSSAPTSQEPAYTLYSLIQPSRKSGSRKRNHSPSFNSTIYE
VIGKSQPKAQNPARLSRKELENFDVYSRVKFSRSADAPAYQQGQNQLYNELNLGRREEYD
VLDKRRGRDPEMGGKPQRRKNPQEGLYNELQKDKMAEAYSEIGMKGERRRGKGHDGLYQ
GLSTATKDTYDALHMQALPPR

In an alternative embodiment, a CAR signaling module comprises the following amino acid sequence:

(SEQ ID NO: 32)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRFWPFLVIIVILSALFLGTLACFCVWRRKRKEKQSETSPKEFLTIYEDVKDLK
TRRNHEQEQTFPGGGSTIYSMIQSQSSAPTSQEPAYTLYSLIQPSRKSGSRKRNHSPSFNS
TIYEVIGKSQPKAQNPARLSRKELENFDVYSRVKFSRSADAPAYQQGQNQLYNELNLGRRE
EYDVLDKRRGRDPEMGGKPQRRKNPQEGLYNELQKDKMAEAYSEIGMKGERRRGKGHD
GLYQGLSTATKDTYDALHMQALPPR

In some embodiments, a CAR signaling module comprises an extracellular NKp46 domain, a transmembrane domain and costimulatory domain form DAP10, and a signaling module from CD3 zeta, according to the following domain arrangement:
NKp46 ECD-DAP10 TM-DAP10 CO1-CD3 zeta signaling module

In a particular embodiment, a CAR signaling module comprises the following amino acid sequence:

(SEQ ID NO: 33)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRLLAGLVAADAVASLLIVGAVFLCARPRRSPAQEDGKVYINMPGRGRVKF
SRSADAPAYQQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPR

In an alternative embodiment, a CAR signaling module comprises the following amino acid sequence:

(SEQ ID NO: 34)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRLPLLAGLVAADAVASLLIVGAVFLCARPRRSPAQEDGKVYINMPGRGRVK
FSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPR

In some embodiments, a CAR signaling module comprises an extracellular NKp46 domain, a transmembrane domain and costimulatory domain form DAP12, and a signaling module from CD3 zeta, according to the following domain arrangement:
NKp46 ECD-DAP12 TM-DAP12 CO1-CD3 zeta signaling module

In a particular embodiment, a CAR signaling module comprises the following amino acid sequence:

(SEQ ID NO: 35)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRGVLAGIVMGDLVLTVLIALAVYFLGRLVPRGRGAAEAATRKQRITETESPY
QELQGQRSDVYSDLNTQRPYYKRVKFSRSADAPAYQQGQNQLYNELNLGRREEYDVLDK
RRGRDPEMGGKPQRRKNPQEGLYNELQKDKMAEAYSEIGMKGERRRGKGHDGLYQGLST
ATKDTYDALHMQALPPR

In an alternative embodiment, a CAR signaling module comprises the following amino acid sequence:

(SEQ ID NO: 36)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRSPGVLAGIVMGDLVLTVLIALAVYFLGRLVPRGRGAAEAATRKQRITETES
PYQELQGQRSDVYSDLNTQRPYYKRVKFSRSADAPAYQQGQNQLYNELNLGRREEYDVLD
KRRGRDPEMGGKPQRRKNPQEGLYNELQKDKMAEAYSEIGMKGERRRGKGHDGLYQGL
STATKDTYDALHMQALPPR

In some embodiments, a CAR signaling module comprises an extracellular NKp46 domain, a transmembrane domain and costimulatory domain from CD28, and a signaling module from CD3 zeta, according to the following domain arrangement:
NKp46 ECD-CD28 TM-CD28 CO1-CD3 zeta signaling module

In a particular embodiment, a CAR signaling module comprises the following amino acid sequence:

(SEQ ID NO: 37)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRFWVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRREEYDVLDKRRGR
DPEMGGKPQRRKNPQEGLYNELQKDKMAEAYSEIGMKGERRRGKGHDGLYQGLSTATKD
TYDALHMQALPPR

In an alternative embodiment, a CAR signaling module comprises the following amino acid sequence:

(SEQ ID NO: 38)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRPFWVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLGRREEYDVLDKRRG
RDPEMGGKPQRRKNPQEGLYNELQKDKMAEAYSEIGMKGERRRGKGHDGLYQGLSTATK
DTYDALHMQALPPR

In some embodiments, a CAR signaling module comprises an extracellular NKp46 domain, a transmembrane domain and a first costimulatory domain from CD28, a second costimulatory domain from 4-1BB and a signaling module from CD3 zeta, according to the following domain arrangement:
NKp46 ECD-CD28 TM-CD28 CO1-4-1BB CO2-CD3 zeta signaling module

In a particular embodiment, a CAR signaling module comprises the following amino acid sequence:

(SEQ ID NO: 39)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRFWVLVVVGGVLACYSLLVTVAFIIFWVRSKRSRLLHSDYMNMTPRRPGP
TRKHYQPYAPPRDFAAYRSKRGRKKLLYIFKQPFMRPVQTTQEEDGCSCRFPEEEEGGCE
LRVKFSRSADAPAYQQGQNQLYNELNLGRREEYDVLDKRRGRDPEMGGKPQRRKNPQEG
LYNELQKDKMAEAYSEIGMKGERRRGKGHDGLYQGLSTATKDTYDALHMQALPPR

In an alternative embodiment, a CAR signaling module comprises the following amino acid sequence:

(SEQ ID NO: 40)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRPFWVLVVVGGVLACYSLLVTVAFIIFWVRSKRSRLLHSDYMNMTPRRPG
PTRKHYQPYAPPRDFAAYRSKRGRKKLLYIFKQPFMRPVQTTQEEDGCSCRFPEEEEGGC
ELRVKFSRSADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHMQALPPR

In some embodiments, a CAR signaling module comprises an extracellular NKp46 domain, a transmembrane domain and costimulatory domain from 4-1EE and a signaling module from CD3 zeta, according to the following domain arrangement:
NKp46 ECD-4-1 BB TM-4-11BE CO1-CD3 zeta signaling module

In a particular embodiment, a CAR signaling module comprises the following amino acid sequence:

(SEQ ID NO: 41)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRIISFFLALTSTALLFLLFFLTLRFSVVKRGRKKLLYIFKQPFMRPVQTTQEE
DGCSCRFPEEEEGGCELRVKFSRSADAPAYQQGQNQLYNELNLGRREEYDVLDKRRGRD
PEMGGKPQRRKNPQEGLYNELQKDKMAEAYSEIGMKGERRRGKGHDGLYQGLSTATKDT
YDALHMQALPPR

In some embodiments, a CAR signaling module comprises an extracellular NKp46 domain, a transmembrane domain from CD8 alpha, a costimulatory domain from CD28 and a signaling module from CD3 zeta, according to the following domain arrangement:
NKp46 ECD-CD8 alpha TM-CD28 CO1-CD3 zeta signaling module

In a particular embodiment, a CAR signaling module comprises the following amino acid sequence:

(SEQ ID NO: 42)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRIYIWAPLAGTCGVLLLSLVITRSKRSRLLHSDYMNMTPRRPGPTRKHYQP
YAPPRDFAAYRSRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLSTATKDTYDALH
MQALPPR

In some embodiments, a CAR signaling module comprises an extracellular NKp46 domain, a transmembrane domain from CD8 alpha, a costimulatory domain from 4-1BB and a signaling module from CD3 zeta, according to the following domain arrangement:
NKp46 ECD-CD8 alpha TM-4-1 BB CO1-CD3 zeta signaling module

In a particular embodiment, a CAR signaling module comprises the following amino acid sequence:

(SEQ ID NO: 43)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRIYIWAPLAGTCGVLLLSLVITKRGRKKLLYIFKQPFMRPVQTTQEEDGCSC
RFPEEEEGGCELRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLSTATKDTYDALH
MQALPPR

In some embodiments, a CAR signaling module comprises an extracellular NKp46 domain, a transmembrane domain from CD8 alpha, a first costimulatory domain from CD28, a second costimulatory domain from 4-1BB and a signaling module from CD3 zeta, according to the following domain arrangement:
NKp46 ECD-CD8 alpha TM-CD28 CO1-4-1BB CO2-CD3 zeta signaling module

In a particular embodiment, a CAR signaling module comprises the following amino acid sequence:

(SEQ ID NO: 44)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRIYIWAPLAGTCGVLLLSLVITRSKRSRLLHSDYMNMTPRRPGPTRKHYQP
YAPPRDFAAYRSKRGRKKLLYIFKQPFMRPVQTTQEEDGCSCRFPEEEEGGCELRVKFSRS
ADAPAYQQGQNQLYNELNLGRREEYDVLDKRRGRDPEMGGKPQRRKNPQEGLYNELQKD
KMAEAYSEIGMKGERRRGKGHDGLYQGLSTATKDTYDALHMQALPPR

In some embodiments, a CAR signaling module comprises an extracellular NKp46 domain, a transmembrane domain and costimulatory domain from OX40 and a signaling module from 25 CD3 zeta, according to the following domain arrangement:
NKp46 ECD-OX40 TM-OX40 CO1-CD3 zeta signaling module

In a particular embodiment, a CAR signaling module comprises the following amino acid sequence:

(SEQ ID NO: 45)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRVAAILGLGLVLGLLGPLAILLALYLLRRDQRLPPDAHKPPGGGSFRTPIQE
EQADAHSTLAKIRVKFSRSADAPAYQQGQNQLYNELNLGRREEYDVLDKRRGRDPEMGGK
PQRRKNPQEGLYNELQKDKMAEAYSEIGMKGERRRGKGHDGLYQGLSTATKDTYDALHM
QALPPR

In some embodiments, a CAR signaling module comprises an extracellular NKp46 domain, a transmembrane domain and a first costimulatory domain from OX40, a second costimulatory domain from 4-1BB and a signaling module from CD3 zeta, according to the following domain arrangement:
NKp46 ECD-OX40 TM-OX40 CO1-4-1BB CO2-CD3 zeta signaling module

In a particular embodiment, a CAR signaling module comprises the following amino acid sequence:

(SEQ ID NO: 46)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRVAAILGLGLVLGLLGPLAILLALYLLRRDQRLPPDAHKPPGGGSFRTPIQE
EQADAHSTLAKIKRGRKKLLYIFKQPFMRPVQTTQEEDGCSCRFPEEEEGGCELRVKFSRS
ADAPAYQQGQNQLYNELNLGRREEYDVLDKRRGRDPEMGGKPQRRKNPQEGLYNELQKD
KMAEAYSEIGMKGERRRGKGHDGLYQGLSTATKDTYDALHMQALPPR

In some embodiments, a CAR signaling module comprises an extracellular NKp46 domain, a transmembrane domain from CD8 alpha, a costimulatory domain from OX40 and a signaling module from CD3 zeta, according to the following domain arrangement:
NKp46 ECD-CD8 alpha TM-OX40 CO1-CD3 zeta signaling module

In a particular embodiment, a CAR signaling module comprises the following amino acid sequence:

(SEQ ID NO: 47)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRIYIWAPLAGTCGVLLLSLVITALYLLRRDQRLPPDAHKPPGGGSFRTPIQE
EQADAHSTLAKIRVKFSRSADAPAYQQGQNQLYNELNLGRREEYDVLDKRRGRDPEMGGK
PQRRKNPQEGLYNELQKDKMAEAYSEIGMKGERRRGKGHDGLYQGLSTATKDTYDALHM
QALPPR

In some embodiments, a CAR signaling module comprises an extracellular NKp46 domain, a transmembrane domain from CD8 alpha, a first costimulatory domain from CD28, a second costimulatory domain from 4-1EE and a signaling module from CD3 zeta, according to the following domain arrangement:
NKp46 ECD-CD8 alpha TM-OX40 CO1-4-1BB CO2-CD3 zeta signaling module

In a particular embodiment, a CAR signaling module comprises the following amino acid sequence:

(SEQ ID NO: 48)
MSSTLPALLCVGLCLSQRISAQQQTLPKPFIWAEPHFMVPKEKQVTICCQGNYGAVEYQLHF
EGSLFAVDRPKPPERINKVKFYIPDMNSRMAGQYSCIYRVGELWSEPSNLLDLVVTEMYDTP
TLSVHPGPEVISGEKVTFYCRLDTATSMFLLLKEGRSSHVQRGYGKVQAEFPLGPVTTAHR
GTYRCFGSYNNHAWSFPSEPVKLLVTGDIENTSLAPEDPTFPADTWGTYLLTTETGLQKDH
ALWDHTAQNLLRIYIWAPLAGTCGVLLLSLVITALYLLRRDQRLPPDAHKPPGGGSFRTPIQE
EQADAHSTLAKIKRGRKKLLYIFKQPFMRPVQTTQEEDGCSCRFPEEEEGGCELRVKFSRS
ADAPAYQQGQNQLYNELNLGRREEYDVLDKRRGRDPEMGGKPQRRKNPQEGLYNELQKD
KMAEAYSEIGMKGERRRGKGHDGLYQGLSTATKDTYDALHMQALPPR

### B) Potentiator Module

The CAR potentiator module proteins that can be used together with cell made to express the CAR signaling module comprise at least (1) a ECD binding moiety capable of binding to the extracellular domain of the CAR signaling module and (2) a moiety that binds an antigen of interest on a target cell (e.g. a target cell that is to be eliminated). For example, the moiety that binds an antigen of interest can be a moiety that binds a tumor antigen. The moiety (1) can also be referred to as the ECD binding moiety.

### (1) ECD Binding Moiety

When the extracellular domain of a CAR signaling module is an ECD of an NCR protein, or a fragment thereof, the ECD binding moiety of the CAR potentiator module is an anti-NCR binding domain, e.g. an antigen binding domain that specifically binds to the particular human NCR polypeptide. In particular embodiment, when the extracellular domain of a CAR signaling module is an ECD of a NKp46 protein, or a fragment thereof, the ECD binding moiety of the CAR potentiator module is an anti-NKp46 binding domain, e.g. an antigen binding domain that specifically binds to human NKp46 polypeptide. In some embodiments, the ECD binding moiety of the CAR potentiator module (or the anti-NKp46 binding domain) binds the D1 domain of NKp46, the D2 domain of NKp46, or binds a region spanning the D1 and D2 domains (at the border of the D1 and D2 domains, the D1/D2 junction), of the NKp46 polypeptide of SEQ ID NO: 1. In some embodiments, the ECD binding moiety of the CAR potentiator module (or the anti-NKp46 binding domain) comprises an immunoglobulin or non-immunoglobulin scaffold, optionally a VH and/or VL domain (e.g. a VHH domain or a VH/VL pair from an anti-NKp46 antibody), having an affinity for human NKp46, as a full-length IgG antibody, characterized by a KD of less than 10⁻⁸ M, less than 10⁻⁹ M, or less than 10⁻¹⁰ M. In some embodiments, the potentiator module protein (or the NKp46-binding ABD thereof) has an affinity (KD) for human NKp46 of between 1 and 100 nM, optionally between 1 and 50 nM, optionally between 1 and 20 nM, optionally about 10 or 15 nM, as determined by SPR. In some embodiments, the ECD binding moiety of the CAR potentiator module binds NKp46 at substantially the same region, site or epitope on NKp46 as antibody NKp46-1, NKp46-2, NKp46-3, NKp46-4, NKp46-6 or NKp46-9. In another embodiment, the antibodies at least partially overlaps, or includes at least one residue in the segment or epitope bound by NKp46-1, NKp46-2, NKp46-3, NKp46-4, NKp46-6 or NKp46-9. In one embodiment, all key residues of the epitope are in a segment corresponding to domain D1 or D2. In one embodiment, the antibody or ECD binding moiety of the CAR potentiator module binds a residue present in the D1 domain as well as a residue present in in the D2 domain. In some embodiments, the antibodies bind an epitope comprising 1, 2, 3, 4, 5, 6, 7 or more residues in the segment corresponding to domain D1 or D2 of the NKp46 polypeptide of SEQ ID NO: 1. In some embodiments, the antibodies bind domain D1 and further bind an epitope comprising 1, 2, 3, or 4 of the residues R101, V102, E104 and/or L105. In other embodiments, the antibodies or ECD binding moiety of the CAR potentiator module bind NKp46 at the D1/D2 domain junction and bind an epitope comprising or consisting of 1, 2, 3, 4 or 5 of the residues K41, E42, E119, Y121 and/or L105. In other embodiments, the antibodies or ECD binding moiety of the CAR potentiator module bind domain D2 and bind an epitope comprising 1, 2, 3 or 4 of the residue P132, E133, I135 and/or S136.

The amino acid sequence of the heavy chain variable region of antibodies NKp46-1, NKp46-2, NKp46-3, NKp46-4, NKp46-6 and NKp46-9 are listed herein in Table B (SEQ ID NOS: 135, 137, 139, 141, 143, 145 respectively), the amino acid sequence of the light chain variable region of antibodies NKp46-1, NKp46-2, NKp46-3, NKp46-4, NKp46-6 and NKp46-9 are also listed herein in Table B (SEQ ID NOS: 136, 138, 140, 142, 144, 146 respectively). Other NKp46 ECD binding VH/VL pairs, CDRs and proteins comprising them (e.g. multispecific NK cell engager proteins) are disclosed in WO2018/0147154, WO2022/212470, WO2022/216723 and NKp46 ECD-binding VHH domains and CDRs thereof are disclosed in WO2023/034741 (e.g. SEQ ID NOS 1-27) and Lipinski et al. (2023) Protein Sci. 32(3):e4593; NKp30 ECD-binding CDRs, VHH domains and VH/VL pairs are disclosed in WO2022/268857 and WO2021/217085; all of which disclosures and sequences are incorporated herein by reference.

In some embodiments, an ECD binding moiety of the CAR potentiator module that binds NKp46 binds essentially the same epitope or determinant as monoclonal antibody NKp46-1, NKp46-2, NKp46-3, NKp46-4, NKp46-6 or NKp46-9; optionally the antibody or ECD binding moiety comprises a hypervariable region of antibody NKp46-1, NKp46-2, NKp46-3, NKp46-4, NKp46-6 or NKp46-9. In any of the embodiments herein, antibody NKp46-1, NKp46-2, NKp46-3, NKp46-4, NKp46-6 or NKp46-9 can be characterized by its amino acid sequence and/or nucleic acid sequence encoding it. In some embodiments, the antibody or ECD binding moiety comprises a Fab or F(ab′)2 portion of NKp46-1, NKp46-2, NKp46-3, NKp46-4, NKp46-6 or NKp46-9. In some embodiments, the ECD binding moiety comprises one, two or three of the CDRs of the heavy and/or light chain variable regions of NKp46-1, NKp46-2, NKp46-3, NKp46-4, NKp46-6 or NKp46-9. Optionally any one or more of said light or heavy chain CDRs may contain one, two, three, four or five or more amino acid modifications (e.g. substitutions, insertions or deletions).

A ECD binding moiety of the CAR potentiator module (or an anti-NKp46 binding domain) can for example comprise:
(a) a heavy chain variable region having the H-CDRs of NKp46-1, NKp46-2, NKp46-3, NKp46-4, NKp46-6 or NKp46-9 as set forth herein, optionally wherein one, two, three or more amino acids may be substituted by a different amino acid;
(b) a light chain variable region having the L-CDRs NKp46-1, NKp46-2, NKp46-3, NKp46-4, NKp46-6 or NKp46-9 as set forth herein, optionally wherein one, two, three or more amino acids may be substituted by a different amino acid;
(c) a heavy chain variable region having the H-CDRs of NKp46-1, NKp46-2, NKp46-3, NKp46-4, NKp46-6 or NKp46-9 as set forth herein, optionally wherein one or more of these amino acids may be substituted by a different amino acid; and a light chain variable region having the L-CDRs of NKp46-1, NKp46-2, NKp46-3, NKp46-4, NKp46-6 or NKp46-9 as set forth herein, optionally wherein one, two, three or more amino acids may be substituted by a different amino acid;
(d) the heavy chain CDR 1, 2 and 3 (HCDR1, HCDR2) amino acid sequence of NKp46-1, NKp46-2, NKp46-3, NKp46-4, NKp46-6 or NKp46-9 as shown in Table A, optionally wherein one, two, three or more amino acids in a CDR may be substituted by a different amino acid;
(e) the light chain CDR 1, 2 and 3 (LCDR1, LCDR2, LCDR3) amino acid sequence of NKp46-1, NKp46-2, NKp46-3, NKp46-4, NKp46-6 or NKp46-9 as shown in Table A, optionally wherein one, two, three or more amino acids in a CDR may be substituted by a different amino acid; or
(f) the heavy chain CDR 1, 2 and 3 (HCDR1, HCDR2, HCDR3) amino acid sequence of NKp46-1, NKp46-2, NKp46-3, NKp46-4, NKp46-6 or NKp46-9 as shown in Table A, optionally wherein one, two, three or more amino acids in a CDR may be substituted by a different amino acid; and the light chain CDRs 1, 2 and 3 (LCDR1, LCDR2, LCDR3) amino acid sequence of the respective NKp46-1, NKp46-2, NKp46-3, NKp46-4, NKp46-6 or NKp46-9 antibody as shown in Table A, optionally wherein one, two, three or more amino acids in a CDR may be substituted by a different amino acid.

In some embodiments, the aforementioned CDRs are according to Kabat, e.g. as shown in Table A. In some embodiments, the aforementioned CDRs are according to Chothia numbering, e.g. as shown in Table A. In some embodiments, the aforementioned CDRs are according to IMGT numbering, e.g. as shown in Table A.

In some embodiments, any of the CDR1, CDR2 and CDR3 of the heavy and light chains may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs listed in the corresponding SEQ ID NO or Table A.

In another embodiments, a ECD binding moiety of the CAR potentiator module (or an anti-NKp46 binding domain) competes for binding to an epitope on NKp46 with a monoclonal antibody according to (a) to (f), above.

The sequences of the CDRs, according to IMGT, Kabat and Chothia definitions systems, are summarized in Table A below and variable regions are shown in Table B below. In any embodiment herein, a VL or VH sequence can be specified or numbered so as to contain or lack a signal peptide or any part thereof.

TABLE A

| | | HCDR1 | | HCDR2 | | HCDR3 | |
|---|---|---|---|---|---|---|---|
| | CDR | SEQ | | SEQ | | SEQ | |
| mAb | definition | ID | Sequence | ID | Sequence | ID | Sequence |
| NKp46-1 | Kabat | 49 | DYVIN | 52 | EIYPGSGTNYYNEK | 55 | RGRYGLYAMDY |
| | | | | | FKA | | |
| | Chothia | 50 | GYTFTDY | 53 | PGSG | 56 | GRYGLYAMD |
| | IMGT | 51 | GYTFTDYV | 54 | IYPGSGTN | 57 | ARRGRYGLYAMD |
| | | | | | | | Y |
| NKp46-2 | Kabat | 58 | SDYAWN | 61 | YITYSGSTSYNPSL | 63 | GGYYGSSWGVFA |
| | | | | | ES | | Y |
| | Chothia | 59 | GYSITSDY | | YSG | 64 | GYYGSSWGVFA |
| | IMGT | 60 | GYSITSDYA | 62 | ITYSGST | 65 | ARGGYYGSSWGV |
| | | | | | | | FAY |
| NKp46-3 | Kabat | 66 | EYTMH | 69 | GISPNIGGTSYNQK | 71 | RGGSFDY |
| | | | | | FKG | | |
| | Chothia | 67 | GYTFTEY | | PNIG | 72 | GGSFD |
| | IMGT | 68 | GYTFTEYT | 70 | ISPNIGGT | 73 | ARRGGSFDY |
| NKp46-4 | Kabat | 74 | SFTMH | 77 | YINPSSGYTEYNQKE | 79 | GSSRGFDY |
| | | | | | KD | | |
| | Chothia | 75 | GYTFTSF | | PSSG | 80 | SSRGED |
| | IMGT | 76 | GYTFTSFT | 78 | INPSSGYT | 81 | VRGSSRGFDY |
| NKp46-6 | Kabat | 82 | SSWMH | 85 | HIHPNSGISNYNEK | 87 | GGREDD |
| | | | | | FKG | | |
| | Chothia | 83 | GYTFTSS | | PNSG | | GRED |
| | IMGT | 84 | GYTFTSSW | 86 | IHPNSGIS | 88 | ARGGREDD |
| NKp46-9 | Kabat | 89 | SDYAWN | 92 | YITYSGSTNYNPSL | 94 | CWDYALYAMDC |
| | | | | | KS | | |
| | Chothia | 90 | GYSITSDY | | YSG | 95 | WDYALYAMD |
| | IMGT | 91 | GYSITSDYA | 93 | ITYSGST | 96 | ARCWDYALYAMD |
| | | | | | | | C |
| NKp46-1 | Kabat | 97 | RASQDISNYLN | 100 | YTSRLHS | 101 | QQGNTRPWT |
| | Chothia | 98 | SQDISNY | | YTS | 102 | GNTRPW |
| | IMGT | 99 | QDISNY | | YTS | 103 | QQGNTRPWT |
| NKp46-2 | Kabat | 104 | RVSENIYSYLA | 107 | NAKTLAE | 108 | QHHYGTPWT |
| | Chothia | 105 | SENIYSY | | NAK | 109 | HYGTPW |
| | IMGT | 106 | ENIYSY | | NAK | 110 | QHHYGTPWT |
| NKp46-3 | Kabat | 111 | RASQSISDYLH | 114 | YASQSIS | 115 | QNGHSFPLT |
| | Chothia | 112 | SQSISDY | | YAS | 116 | GHSFPL |
| | IMGT | 113 | QSISDY | | YAS | 115 | QNGHSFPLT |
| NKp46-4 | Kabat | 117 | RASENIYSNLA\| | 120 | AATNLAD | 121 | QHFWGTPRT |
| | Chothia | 118 | SENIYSN | | AAT | 122 | FWGTPR |
| | IMGT | 119 | ENIYSN | | AAT | 121 | QHFWGTPRT |
| NKp46-6 | Kabat | 123 | RASQDIGSSLN | 126 | ATSSLDS | 127 | LQYASSPWT |
| | Chothia | 124 | SQDIGSS | | ATS | 128 | YASSPWT |
| | IMGT | 125 | QDIGSS | | ATS | 129 | LQYASSPWT |
| NKp46-9 | Kabat | 130 | RTSENIYSYLA | 131 | NAKTLAE | 132 | QHHYDTPLT |
| | Chothia | 105 | SENIYSY | | NAK | 133 | HYDTPL |
| | IMGT | 106 | ENIYSY | | NAK | 132 | QHHYDTPLT |

TABLE B

| | SEQ ID | |
|---|---|---|
| Antibody | NO | Amino acid sequence |
| NKp46-1 VH | 134 | QVQLQQSGPELVKPGASVKMSCKASGYTFTDYVINWGKQRSGQG |
| | | LEWIGEIYPGSGTNYYNEKFKAKATLTADKSSNIAYMQLSSLTSEDS |
| | | AVYFCARRGRYGLYAMDYWGQGTSVTVSS |
| NKp46-1 VL | 135 | DIQMTQTTSSLSASLGDRVTISCRASQDISNYLNWYQQKPDGTVKL |
| | | LIYYTSRLHSGVPSRFSGSGSGTDYSLTINNLEQEDIATYFCQQGNT |
| | | RPWTFGGGTKLEIK |
| NKp46-2 VH | 136 | EVQLQESGPGLVKPSQSLSLTCTVTGYSITSDYAWNWIRQFPGNKL |
| | | EWMGYITYSGSTSYNPSLESRISITRDTSTNQFFLQLNSVTTEDTAT |
| | | YYCARGGYYGSSWGVFAYWGQGTLVTVSA |
| NKp46-2 VL | 137 | DIQMTQSPASLSASVGETVTITCRVSENIYSYLAWYQQKQGKSPQL |
| | | LVYNAKTLAEGVPSRFSGSGSGTQFSLKINSLQPEDFGSYYCQHHY |
| | | GTPWTFGGGTKLEIK |
| NKp46-3 VH | 138 | EVQLQQSGPELVKPGASVKISCKTSGYTFTEYTMHWVKQSHGKSL |
| | | EWIGGISPNIGGTSYNQKFKGKATLTVDKSSSTAYMELRSLTSEDSA |
| | | VYYCARRGGSFDYWGQGTTLTVSS |
| NKp46-3 VL | 139 | DIVMTQSPATLSVTPGDRVSLSCRASQSISDYLHWYQQKSHESPRL |
| | | LIKYASQSISGIPSRFSGSGSGSDFTLSINSVEPEDVGVYYCQNGHS |
| | | FPLTFGAGTKLELK |
| NKp46-4 VH | 140 | QVQLQQSAVELARPGASVKMSCKASGYTFTSFTMHWVKQRPGQG |
| | | LEWIGYINPSSGYTEYNQKFKDKTTLTADKSSSTAYMQLDSLTSDD |
| | | SAVYYCVRGSSRGFDYWGQGTLVTVSA |
| NKp46-4 VL | 141 | DIQMIQSPASLSVSVGETVTITCRASENIYSNLAWFQQKQGKSPQLL |
| | | VYAATNLADGVPSRFSGSGSGTQYSLKINSLQSEDFGIYYCQHFW |
| | | GTPRTFGGGTKLEIK |
| NKp46-6 VH | 142 | QVQLQQPGSVLVRPGASVKLSCKASGYTFTSSWMHWAKQRPGQ |
| | | GLEWIGHIHPNSGISNYNEKFKGKATLTVDTSSSTAYVDLSSLTSED |
| | | SAVYYCARGGRFDDWGAGTTVTVSS |
| NKp46-6 VL | 143 | DIQMTQSPSSLSASLGERVSLTCRASQDIGSSLNWLQQEPDGTIKR |
| | | LIYATSSLDSGVPKRFSGSRSGSDYSLTISSLESEDFVDYYCLQYAS |
| | | SPWTFGGGTKLEIK |
| NKp46-9 VH | 144 | DVQLQESGPGLVKPSQSLSLTCTVTGYSITSDYAWNWIRQFPGNKL |
| | | EWMGYITYSGSTNYNPSLKSRISITRDTSKNQFFLQLNSVTTEDTAT |
| | | YYCARCWDYALYAMDCWGQGTSVTVSS |
| NKp46-9 VL | 145 | DIQMTQSPASLSASVGETVTITCRTSENIYSYLAWCQQKQGKSPQL |
| | | LVYNAKTLAEGVPSRFSGSGSGTHFSLKINSLQPEDFGIYYCQHHY |
| | | DTPLTFGAGTKLELK |

VH and VL pairs of a ECD binding moiety of the CAR potentiator module that binds NKp46 (or a NKp46 binding domain) can optionally be function-conservative variants of the VH and VL of any of antibodies NKp46-1, NKp46-2, NKp46-3, NKp46-4, NKp46-6 or NKp46-9. “Function-conservative variants” are those in which a given amino acid residue in a protein (e.g. an antibody or antibody fragment) has been changed without altering the overall conformation and function of the protein, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity with the antibody capable of specifically binding to a NKp46 polypeptide as defined hereinabove as determined by BLAST or FASTA algorithms, preferably at least 75%, more preferably at least 85%, still preferably at least 90%, and even more preferably at least 95%, and which has the same or substantially similar properties or functions as the antibodies capable of specifically binding to a NKp46 polypeptide as defined hereinabove.

Exemplary humanized VH and VL domains can comprise all of an antigen binding region of antibody NKp46-1, NKp46-2, NKp46-3, NKp46-4, NKp46-6 or NKp46-9, for example having the amino acids of the SEQ ID NOS shown in Table C hereinafter.

A light chain variable region of a NKp46-1, NKp46-2, NKp46-3, NKp-46-4, NKp46-6 or NKp46-9 antibody may comprise, for the respective antibody: a human light chain FR1 framework region; a LCDR1 region comprising an amino acid sequence as set forth in Table A, or a sequence of at least 4, 5, 6, 7, 8, 9 or 10 contiguous amino acids thereof, wherein one or more of these amino acids may be substituted by a different amino acid; a human light chain FR2 framework region; a LCDR2 region comprising an amino acid sequence as set forth in Table A, or a sequence of at least 4, 5, 6, 7, 8, 9 or 10 contiguous amino acids thereof, wherein one or more of these amino acids may be substituted by a different amino acid; a human light chain FR3 framework region; and a LCDR3 region comprising an amino acid sequence as set forth in Table A, or a sequence of at least 4, 5, 6, 7, 8, 9 or 10 contiguous amino acids thereof, wherein one or more of these amino acids may be deleted or substituted by a different amino acid. Optionally, the variable region further comprises a human light chain FR4 framework region. Humanization of NKp46-1, NKp46-2, NKp46-3, NKp-46-4, and NKp46-9 VH/VL domains is described in PCT publication no. WO2017114694, the disclosure of which is incorporated herein by reference, and amino acid sequences are shown below.

NKp46-1: “H1” heavy chain variable region
(SEQ ID NO: 146)
QVQLVQSGAEVKKPGSSVKVSCKASGYTFSDYVINWVRQAPGQGLEWMGEIYPGSGTNYY
NEKFKAKATITADKSTSTAYMELSSLRSEDTAVYYCARRGRYGLYAMDYWGQGTTVTVSS
NKp46-1: “H3” heavy chain variable region
(SEQ ID NO: 147)
QVQLVQSGAEVKKPGSSVKVSCKASGYTFTDYVINWGRQAPGQGLEWIGEIYPGSGTNYY
NEKFKAKATITADKSTSTAYMELSSLRSEDTAVYECARRGRYGLYAMDYWGQGTTVTVSS
NKp46-1: “L1” light chain variable region
(SEQ ID NO: 148)
DIQMTQSPSSLSASVGDRVTITCRASQDISNYLNWYQQKPGKAPKLLIYYTSRLHSGVPSRF
SGSGSGTDFTFTISSLQPEDIATYFCQQGNTRPWTFGGGTKVEIK
NKp46-2: “H1” heavy chain variable region
(SEQ ID NO: 149)
QVQLQESGPGLVKPSQTLSLTCTVSGYSISSDYAWNWIRQPPGKGLEWIGYITYSGSTSYN
PSLESRVTISRDTSKNQFSLKLSSVTAADTAVYYCARGGYYGSSWGVFAYWGQGTLVTVSS
NKp46-2: “H2” heavy chain variable region
(SEQ ID NO: 150)
QVQLQESGPGLVKPSQTLSLTCTVSGYSISSDYAWNWIRQPPGKGLEWMGYITYSGSTSYN
PSLESRITISRDTSKNQFSLKLSSVTAADTAVYYCARGGYYGSSWGVFAYWGQGTLVTVSS
NKp46-2: “H3” heavy chain variable region
(SEQ ID NO: 151)
QVQLQESGPGLVKPSQTLSLTCTVSGYSITSDYAWNWIRQPPGKGLEWMGYITYSGSTSYN
PSLESRITISRDTSKNQFSLKLSSVTAADTAVYYCARGGYYGSSWGVFAYWGQGTLVTVSS
NKp46-2: “L1” light chain variable region
(SEQ ID NO: 152)
DIQMTQSPSSLSASVGDRVTITCRVSENIYSYLAWYQQKPGKAPKLLVYNAKTLAEGVPSRF
SGSGSGTDFTLTISSLQPEDFATYYCQHHYGTPWTFGGGTKVEIK
NKp46-3: “H1” heavy chain variable region
(SEQ ID NO: 153)
QVQLVQSGAEVKKPGSSVKVSCKASGYTFSEYTMHWVRQAPGQGLEWMGGISPNIGGTS
YNQKFKGRVTITADKSTSTAYMELSSLRSEDTAVYYCARRGGSFDYWGQGTTVTVSS
NKp46-3: “H3” heavy chain variable region
(SEQ ID NO: 154)
QVQLVQSGAEVKKPGSSVKVSCKASGYTFSEYTMHWVRQAPGQGLEWIGGISPNIGGTSY
NQKFKGRATITADKSTSTAYMELSSLRSEDTAVYYCARRGGSFDYWGQGTTVTVSS
NKp46-3: “H4” heavy chain variable region
(SEQ ID NO: 155)
QVQLVQSGAEVKKPGSSVKVSCKASGYTFSEYTMHWVRQAPGQGLEWIGGISPNIGGTSY
NQKFKGRATLTADKSTSTAYMELSSLRSEDTAVYYCARRGGSFDYWGQGTTVTVSS
NKp46-3: “L1” light chain variable region
(SEQ ID NO: 156)
EIVMTQSPATLSVSPGERATLSCRASQSISDYLHWYQQKPGQAPRLLIKYASQSISGIPARFS
GSGSGTDFTLTISSLEPEDFAVYYCQNGHSFPLTFGQGTKLEIK
NKp46-4: “H1” heavy chain variable region
(SEQ ID NO: 157)
QVQLVQSGAEVKKPGASVKVSCKASGYTFTSFTMHWVRQAPGQGLEWIGYINPSSGYTEY
NQKFKDRVTITADKSTSTAYMELSSLRSEDTAVYYCVRGSSRGFDYWGQGTLVTVSS
NKp46-4: “H2” heavy chain variable region
(SEQ ID NO: 158)
QVQLVQSGAEVKKPGASVKVSCKASGYTFTSFTMHWVRQAPGQGLEWIGYINPSSGYTEY
NQKFKDRTTITADKSTSTAYMELSSLRSEDTAVYYCVRGSSRGFDYWGQGTLVTVSS
NKp46-4: “H3” heavy chain variable region
(SEQ ID NO: 159)
QVQLVQSGAEVKKPGASVKVSCKASGYTFTSFTMHWVRQAPGQGLEWIGYINPSSGYTEY
NQKFKDRITLTADKSTSTAYMELSSLRSEDTAVYYCVRGSSRGFDYWGQGTLVTVSS
NKp46-4: “L2” light chain variable region
(SEQ ID NO: 160)
DIQMTQSPSSLSASVGDRVTITCRASENIYSNLAWEQQKPGKAPKLLVYAATNLADGVPSRF
SGSGSGTDYTLTISSLQPEDFATYYCQHFWGTPRTFGGGTKVEIK
NKp46-9: “H1” heavy chain variable region
(SEQ ID NO: 161)
QVQLQESGPGLVKPSQTLSLTCTVSGGSISSDYAWNWIRQPPGKGLEWIGYITYSGSTNYN
PSLKSRVTISRDTSKNQFSLKLSSVTAADTAVYYCARCWDYALYAMDCWGQGTTVTVSS
NKp46-9: “H2” heavy chain variable region
(SEQ ID NO: 162)
QVQLQESGPGLVKPSQTLSLTCTVSGYSISSDYAWNWIRQPPGKGLEWIGYITYSGSTNYN
PSLKSRVTISRDTSKNQFSLKLSSVTAADTAVYYCARCWDYALYAMDCWGQGTTVTVSS
NKp46-9: ″H3” heavy chain variable region
(SEQ ID NO: 163)
QVQLQESGPGLVKPSQTLSLTCTVSGYSISSDYAWNWIRQPPGKGLEWMGYITYSGSTNY
NPSLKSRITISRDTSKNQFSLKLSSVTAADTAVYYCARCWDYALYAMDCWGQGTTVTVSS
NKp46-9: ″L1” light chain variable region
(SEQ ID NO: 164)
DIQMTQSPSSLSASVGDRVTITCRTSENIYSYLAWCQQKPGKAPKLLIYNAKTLAEGVPSRFS
GSGSGTDFTLTISSLQPEDFATYYCQHHYDTPLTFGQGTKLEIK
NKp46-9: ″L2” light chain variable region
(SEQ ID NO: 165)
DIQMTQSPSSLSASVGDRVTITCRTSENIYSYLAWCQQKPGKAPKLLVYNAKTLAEGVPSRF
SGSGSGTDFTLTISSLQPEDFATYYCQHHYDTPLTFGQGTKLEIK
Examples of VH and VL combinations include:

a VH comprising a CDR1, 2 and 3 of SEQ ID NO: 134 and a FR1, 2 and 3 derived from a human IGHV1-69 gene segment, and a VL comprising a CDR1, 2 and 3 of SEQ ID NO: 135 and a FR1, 2 and 3 derived from a human IGKV1-33 gene segment;
a VH comprising a CDR1, 2 and 3 of SEQ ID NO: 136 and a FR1, 2 and 3 of a human IGHV4-30-4 gene segment, and a VL comprising a CDR1, 2 and 3 of SEQ ID NO: 137 and a FR1, 2 and 3 derived from a human IGKV1-39 gene segment;
a VH comprising a CDR1, 2 and 3 of SEQ ID NO: 138 and a FR1, 2 and 3 derived from a human IGHV1-69 gene segment, and a VL comprising a CDR1, 2 and 3 of SEQ ID NO: 139 and a FR1, 2 and 3 derived from a human IGKV3-11 and/or IGKV3-15 gene segment;
a VH comprising a CDR1, 2 and 3 of SEQ ID NO: 140 and a FR1, 2 and 3 derived from a human IGHV1-46 and/or a IGHV1-69 gene segment, and a VL comprising a CDR1, 2 and 3 of SEQ ID NO: 141 and a FR1, 2 and 3 derived from a human IGKV1-NL1 gene segment; or
a VH comprising a CDR1, 2 and 3 of SEQ ID NO: 144 and a FR1, 2 and 3 derived from a human IGHV4-30-4 gene segment, and a VL comprising a CDR1, 2 and 3 of SEQ ID NO: 145 and a FR1, 2 and 3 derived from a human IGKV1-39 gene segment.

In another aspect, examples of humanized anti-NKp46 VH and VL combinations include:
a VH comprising an amino acid sequence at least 70%, 80%, 90%, 95%, 98% or 100% identical to the amino acid sequence of the NKp46-1 H1 or H3 variable domain, and a VL comprising an amino acid sequence at least 70%, 80%, 90%, 95%, 98% or 100% identical to the amino acid sequence of the NKp46-1 L1 variable domain;
a VH comprising an amino acid sequence at least 70%, 80%, 90%, 95%, 98% or 100% identical to the amino acid sequence of the NKp46-2 H1, H2 or H3 variable domain, and a VL comprising an amino acid sequence at least 70%, 80%, 90%, 95%, 98% or 100% identical to the amino acid sequence of the NKp46-2 L1 variable domain;
a VH comprising an amino acid sequence at least 70%, 80%, 90%, 95%, 98% or 100% identical to the amino acid sequence of the NKp46-3 H1, H3 or H4 variable domain, and a VL comprising an amino acid sequence at least 70%, 80%, 90%, 95%, 98% or 100% identical to the amino acid sequence of the NKp46-3 L1 variable domain;
a VH comprising an amino acid sequence at least 70%, 80%, 90%, 95%, 98% or 100% identical to the amino acid sequence of the NKp46-4 H1 variable domain, and a VL comprising an amino acid sequence at least 70%, 80%, 90%, 95%, 98% or 100% identical to the amino acid sequence of the NKp46-4 L2 variable domain;
a VH comprising an amino acid sequence at least 70%, 80%, 90%, 95%, 98% or 100% identical to the amino acid sequence of the NKp46-9 H2 variable domain, and a VL comprising an amino acid sequence at least 70%, 80%, 90%, 95%, 98% or 100% identical to the amino acid sequence of the NKp46-9 L1 or L2 variable domain; or
a VH comprising an amino acid sequence at least 70%, 80%, 90%, 95%, 98% or 100% identical to the amino acid sequence of the NKp46-9 H3 variable domain, and a VL comprising an amino acid sequence at least 70%, 80%, 90%, 95%, 98% or 100% identical to the amino acid sequence of the NKp46-9 L1 or L2 variable domain.

TABLE C

| Antibody | VH (SEQ ID NO) | VL (SEQ ID NO) |
|---|---|---|
| NKp46-1 H1L1 | 146 | 148 |
| NKp46-1 H3L1 | 147 | 148 |
| NKp46-2 H1L1 | 149 | 152 |
| NKp46-2 H2L1 | 150 | 152 |
| NKp46-2 H3L1 | 151 | 152 |
| NKp46-3 H1L1 | 153 | 154 |
| NKp46-3 H3L1 | 154 | 156 |
| NKp46-3 H4L1 | 155 | 156 |
| NKp46-4 H1L2 | 157 | 160 |
| NKp46-4 H2L2 | 158 | 160 |
| NKp46-4 H3L2 | 159 | 160 |
| NKp46-9 H2L1 | 162 | 164 |
| NKp46-9 H2L2 | 162 | 165 |
| NKp46-9 H3L1 | 163 | 164 |
| NKp46-9 H3L2 | 163 | 165 |

Other examples of anti-NKp46 antigen binding domains include single domains such as a VHH domain comprising the amino acid sequence of SEQ ID NOS: 232 or 233, or a CDR1, 2 and 3 thereof, or an amino acid sequence at least 70%, 80%, 90%, 95%, 98% or 100% identical thereto. Alternatively, antigen binding domains used in the proteins described herein can be derived from any of a variety of non-immunoglobulin scaffolds, for example affibodies based on the Z-domain of staphylococcal protein A, engineered Kunitz domains, monobodies or adnectins based on the 10th extracellular domain of human fibronectin Ill, anticalins derived from lipocalins, DARPins® (designed ankyrin repeat domains, multimerized LDLR-A module, avimers or cysteine-rich knottin peptides. See, e.g., Gebauer and Skerra (2009) Current Opinion in Chemical Biology 13:245-255, the disclosure of which is incorporated herein by reference.

### (2) Tumor Antigen Binding Moiety

In some embodiments, a CAR potentiator module comprises a tumor antigen binding moiety.

Disclosed herein are also chimeric activating receptor potentiator modules comprising at least (1) a ECD binding moiety capable of binding an ECD of a CAR signaling module and (2) a tumor antigen binding moiety. A tumor antigen binding moiety may for example be characterized as an antigen binding domain that binds (e.g. specifically) to a tumor antigen.

As used herein, the terms “tumor antigen” or “cancer antigen” are used interchangeably and refer to antigens (other than NKp46, and CD16) that are differentially expressed by cancer cells or are expressed by non-tumoral cells (e.g. immune cells) having a pro-tumoral effect (e.g. an immunosuppressive effect), and can thereby be exploited in order to target cancer cells. Cancer antigens can be antigens which can potentially stimulate apparently tumor-specific immune responses. Some of these antigens are encoded, although not necessarily expressed, or expressed at lower levels or less frequently, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Still other cancer antigens can be expressed on immune cells capable of contributing to or mediating a pro-tumoral effect, e.g. cell that contributes to immune evasion, a monocyte or a macrophage, optionally a suppressor T cell, regulatory T cell, or myeloid-derived suppressor cell.

The cancer antigens are usually normal cell surface antigens which are either over-expressed or expressed at abnormal times, or are expressed by a targeted population of cells. Ideally the target antigen is expressed only on proliferative cells (e.g., tumor cells) or pro-tumoral cells (e.g. immune cells having an immunosuppressive effect), however this is rarely observed in practice. As a result, target antigens are in many cases selected on the basis of differential expression between proliferative/disease tissue and healthy tissue. Example of cancer antigens include: Receptor Tyrosine Kinase-like Orphan Receptor 1 (ROR1), Crypto, CD4, CD19, CD20, CD30, CD38, CD47, Glycoprotein NMB, CanAg, Her2 (ErbB2/Neu), a Siglec family member, for example CD22 (Siglec2) or CD33 (Siglec3), CD79, CD123, CD138, CD171, PSCA, L1-CAM, PSMA (prostate specific membrane antigen), BCMA, CD52, CD56, CD80, CD70, E-selectin, EphB2, Melanotransferrin, Mud 6 and TMEFF2. Examples of cancer antigens also include Immunoglobulin superfamily (IgSF) such as cytokine receptors, Killer-Ig Like Receptor, CD28 family proteins, for example, Killer-Ig Like Receptor 3DL2 (KIR3DL2), B7-H3, B7-H4, B7-H6, PD-L1. Examples also include MAGE, MART-1/Melan-A, gp100, major histocompatibility complex class I-related chain A and B polypeptides (MICA and MICB), HLA-G, adenosine deaminase-binding protein (ADAbp), cyclophilin b, colorectal associated antigen (CRC)-CO17-1A/GA733, protein tyrosine kinase 7(PTK7), receptor protein tyrosine kinase 3 (TYRO-3), nectins (e.g. nectin-4), major histocompatibility complex class I-related chain A and B polypeptides (MICA and MICB), proteins of the UL16-binding protein (ULBP) family, proteins of the retinoic acid early transcript-1 (RAET1) family, carcinoembryonic antigen (CEA) and its immunogenic epitopes CAP-1 and CAP-2, etv6, aml1, prostate specific antigen (PSA), T-cell receptor/CD3-zeta chain, MAGE-family of tumor antigens, GAGE-family of tumor antigens, anti-Mullerian hormone Type II receptor, delta-like ligand 4 (DLL4), DR5, ROR1 (also known as Receptor Tyrosine Kinase-Like Orphan Receptor 1 or NTRKR1 (EC 2.7.10.1), BAGE, RAGE, LAGE-1, NAG, GnT-V, MUM-1, CDK4, MUC family, VEGF, VEGF receptors, Angiopoietin-2, PDGF, TGF-alpha, EGF, EGF receptor, members of the human EGF-like receptor family, e.g., HER-2/neu, HER-3, HER-4 or a heterodimeric receptor comprised of at least one HER subunit, gastrin releasing peptide receptor antigen, Muc-1, CA125, integrin receptors, αvβ3 integrins, α5β1 integrins, αIIbβ3-integrins, PDGF beta receptor, SVE-cadherin, hCG, CSF1R (tumor-associated monocytes and macrophages), α-fetoprotein, E-cadherin, α-catenin, β-catenin and delta-catenin, p120ctn, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2, although this is not intended to be exhaustive.

By way of examples, when the tumor antigen binding domain of a CAR potentiator module binds to a HER2 polypeptide, exemplary VH and VL pairs can be selected from antibodies trastuzumab, pertuzumab or margetuximab:

### Trastuzumab Heavy Chain Variable Region

(SEQ ID NO: 166)
EVQLVESGGG LVQPGGSLRL SCAASGFNIK DTYIHWVRQA
PGKGLEWVAR IYPTNGYTRY ADSVKGRFTI SADTSKNTAY
LQMNSLRAED TAVYYCSRWG GDGFYAMDYW GQGTLVTVSS.

### Trastuzumab Light Chain Variable Region

(SEQ ID NO: 167)
DIQMTQSPSS LSASVGDRVT ITCRASQDVN TAVAWYQQKP GKAPKLLIYS ASFLYSGVPS
RFSGSRSGTD FTLTISSLQP EDFATYYCQQ HYTTPPTFGQ GTKVEIK.
Margetuximab VH:
(SEQ ID NO: 168)
QVQLQQSGPE LVKPGASLKL SCTASGFNIK DTYIHWVKQR PEQGLEWIGRIYPTNGYTRY
DPKFQDKATI TADTSSNTAY LQVSRLTSED TAVYYCSRWG GDGFYAMDYW
GQGASVTVSS.
Margetuximab VL:
(SEQ ID NO: 169)
DIVMTQSHKF MSTSVGDRVS ITCKASQDVN TAVAWYQQKP GHSPKLLIYS ASFRYTGVPD
RFTGSRSGTD FTFTISSVQA EDLAVYYCQQ HYTTPPTFGG
GTKVEIK

In another example, when the tumor antigen binding domain of a CAR potentiator module binds to a CD19 polypeptide, exemplary VH and VL pairs can be selected from the VH and VL pair from blinatumomab.

Blinatumomab VH:
(SEQ ID NO: 170)
QVQLQQSGAELVRPGSSVKISCKASGYAFSSYWMNWVKQRPGQGLEWIGQIWPGDGDTN
YNGKFKGKATLTADESSSTAYMQLSSLASEDSAVYFCARRETTTVGRYYYAMDYWGQGTT
VTVSS.
Blinatumomab VL:
(SEQ ID NO: 171)
DIQLTQSPASLAVSLGQRATISCKASQSVDYDGDSYLNWYQQIPGQPPKLLIYDASNLVSGIP
PRFSGSGSGTDFTLNIHPVEKVDAATYHCQQSTEDPWTFGGGTKLEIK

In another example, when tumor antigen binding domain of a CAR potentiator module binds to a CD20 polypeptide, exemplary VH and VL pairs can be selected from VH and VL pair from rituximab and obinutuzumab:

Rituximab VH:
(SEQ ID NO: 172)
QVQLQQPGAELVKPGASVKMSCKASGYTFTSYNMHWVKQTPGRGLEWIGAIYPGNGDTSY
NQKFKGKATLTADKSSSTAYMQLSSLTSEDSAVYYCARSTYYGGDWYFNVWGAGTTVTVS
A.
Rituximab VL:
(SEQ ID NO: 173)
QIVLSQSPAILSASPGEKVTMTCRASSSVSYIHWFQQKPGSSPKPWIYATSNLASGVPVRFS
GSGSGTSYSLTISRVEAEDAATYYCQQWTSNPPTFGGGTKLEIK
Obinutuzumab VH:
(SEQ ID NO: 174)
QVQLVQSGAEVKKPGSSVKVSCKASGYAFSYSWINWVRQAPGQGLEWMGRIFPGDGDTD
YNGKFKGRVTITADKSTSTAYMELSSLRSEDTAVYYCARNVFDGYWLVYWGQGTLVTVSS.
Obinutuzumab VL:
(SEQ ID NO: 175)
DIVMTQTPLSLPVTPGEPASISCRSSKSLLHSNGITYLYWYLQKPGQSPQLLIYQMSNLVSGV
PDRFSGSGSGTDFTLKISRVEAEDVGVYYCAQNLELPYTFGGGTKVEIK.

In another example, when tumor antigen binding domain of a CAR potentiator module binds to a EGFR polypeptide, exemplary VH and VL pairs can be selected from the EGFR-binding VH and VL pair from cetuximab, panitumumab, nimotuzumab, depatuxizumab and necitumumab:

Cetuximab VH:
(SEQ ID NO: 176)
QVQLKQSGPGLVQPSQSLSITCTVSGFSLTNYGVHWVRQSPGKGLEWLGVIWSGGNTDYN
TPFTSRLSINKDNSKSQVFFKMNSLQSNDTAIYYCARALTYYDYEFAYWGQGTLVTVSA.
Cetuximab VL:
(SEQ ID NO: 177)
DILLTQSPVILSVSPGERVSFSCRASQSIGTNIHWYQQRTNGSPRLLIKYASESISGIPSRFSG
SGSGTDFTLSINSVESEDIADYYCQQNNNWPTTFGAGTKLELK.
Panitumumab VH:
(SEQ ID NO: 178)
QVQLQESGPGLVKPSETLSLTCTVSGGSVSSGDYYWTWIRQSPGKGLEWIGHIYYSGNTNY
NPSLKSRLTISIDTSKTQFSLKLSSVTAADTAIYYCVRDRVTGAFDIWGQGTMVTVSS.
Panitumumab VL:
(SEQ ID NO: 179)
DIQMTQSPSSLSASVGDRVTITCQASQDISNYLNWYQQKPGKAPKLLIYDASNLETGVPSRF
SGSGSGTDFTFTISSLQPEDIATYFCQHFDHLPLAFGGGTKVEIK.
Nimotuzumab VH:
(SEQ ID NO: 180)
QVQLQQSGAEVKKPGSSVKVSCKASGYTFTNYYIYWVRQAPGQGLEWIGGINPTSGGSNF
NEKFKTRVTITADESSTTAYMELSSLRSEDTAFYFCTRQGLWFDSDGRGFDFWGQGTTVTV
SS
Nimotuzumab VL:
(SEQ ID NO: 181)
DIQMTQSPSSLSASVGDRVTITCRSSQNIVHSNGNTYLDWYQQTPGKAPKLLIYKVSNRFSG
VPSRFSGSGSGTDFTFTISSLQPEDIATYYCFQYSHVPWTFGQGTKLQI.
Necitumumab VH:
(SEQ ID NO: 182)
QVQLQESGPGLVKPSQTLSLTCTVSGGSISSGDYYWSWIRQPPGKGLEWIGYIYYSGSTDY
NPSLKSRVTMSVDTSKNQFSLKVNSVTAADTAVYYCARVSIFGVGTFDYWGQGTLVTVSS.
Necitumumab VL:
(SEQ ID NO: 183)
EIVMTQSPATLSLSPGERATLSCRASQSVSSYLAWYQQKPGQAPRLLIYDASNRATGIPARF
SGSGSGTDFTLTISSLEPEDFAVYYCHQYGSTPLTFGGGTKAEIK.
Depatuxizumab VH:
(SEQ ID NO: 184)
QVQLQESGPGLVKPSQTLSLTCTVSGYSISSDFAWNWIRQPPGKGLEWMGYISYSGNTRY
QPSLKSRITISRDTSKNQFFLKLNSVTAADTATYYCVTAGRGFPYWGQGTLVTVSS.
Depatuxizumab VL:
(SEQ ID NO: 185)
DIQMTQSPSSMSVSVGDRVTITCHSSQDINSNIGWLQQKPGKSFKGLIYHGTNLDDGVPSRF
SGSGSGTDYTLTISSLQPEDFATYYCVQYAQFPWTFGGGTKLEIK.

In another example, when tumor antigen binding domain of a CAR potentiator module binds to a BCMA polypeptide, exemplary VH and VL pairs can be selected from the BCMA-binding VH and VL pair from belantamab, teclistamab, elranatamab or pavurutamab:

Belantamab VH:
(SEQ ID NO: 186)
QVQLVQSGAEVKKPGSSVKVSCKASGGTFSNYWMHWVRQAPGQGLEWMGATYRGHSDT
YYNQKFKGRVTITADKSTSTAYMELSSLRSEDTAVYYCARGAIYDGYDVLDNWGQGTLVTV
SS.
Belantamab VL:
(SEQ ID NO: 187)
DIQMTQSPSSLSASVGDRVTITCSASQDISNYLNWYQQKPGKAPKLLIYYTSNLHSGVPS
RFSGSGSGTDFTLTISSLQPEDFATYYCQQYRKLPWTFGQGTKLEIK.
Pavurutamab VH:
(SEQ ID NO: 188)
QVQLVQSGAEVKKPGASVKVSCKASGYTFTNHIIHWVRQAPGQCLEWMGYINPYPGYHAY
NEKFQGRATMTSDTSTSTVYMELSSLRSEDTAVYYCARDGYYRDTDVLDYWGQGTLVTVS
S
Pavurutamab VL:
(SEQ ID NO: 189)
DIQMTQSPSSLSASVGDRVTITCQASQDISNYLNWYQQKPGKAPKLLIYYTSRLHTGVPSRF
SGSGSGTDFTFTISSLEPEDIATYYCQQGNTLPWTFGCGTKVEIK.

In another example, when the tumor antigen binding domain of a CAR potentiator module binds to a PD-L1 polypeptide, exemplary VH and VL pairs can be selected from the PD-L1-binding VH and VL pair from antibodies 3G10, 12A4, 10A5, 5F8, 10H10, 1B12, 7H1, 11E6, 12B7, and 13G4 shown in U.S. Pat. No. 7,943,743, the disclosure of which is incorporated herein by reference, or of any of the antibodies MPDL3280A (atezolizumab, Tecentriq™, see, e.g., U.S. Pat. No. 8,217,149, anti-PD-L1 from Roche/Genentech), MDX-1105 (anti-PD-L1 from Bristol-Myers Squibb), MSB0010718C (avelumab; anti-PD-L1 from Pfizer) and MED14736 (durvalumab; anti-PD-L1 from AstraZeneca).

In another example, when the tumor antigen binding domain of a CAR potentiator module binds to a B7-H3 polypeptide, exemplary VH and VL pairs can be selected from the B7-H3-binding VH and VL pairs of enoblituzumab, of TRL4542 shown in PCT publication no. WO2018/129090, of 8H9 shown in PCT publication no. WO2018/209346, or of any of the antibodies of PCT publication nos. WO2016/106004, WO2017/180813, WO2019/024911, WO2019/225787, WO2020/063673, WO2020/094120, WO2020/102779, WO2020/140094 and WO2020/151384. Examples of single domain B7H3 ABDs that can be used include Affibody™ formats described in PCT publication WO2020/041626 and single domain antibodies (sdAb) of PCT publication nos. WO2020/076970 and WO2021/247794. The disclosures of VH, VL and CDRs sequences of the above are incorporated herein by reference.

In another example, when the tumor antigen binding domain of a CAR potentiator module binds to a B7-H6 polypeptide, exemplary VH and VL pairs can be selected from the B7-H6-binding VH and VL pairs shown in U.S. Pat. Nos. 11,034,766; 8,822,652; 9,676,855; 11,034,766; 11,034,767 or in PCT publication nos. WO2013/037727 or WO2021/064137.

In another example, when the tumor antigen binding domain of a CAR potentiator module binds to a B7-H4 polypeptide, exemplary VH and VL pairs can be selected from the B7-H4-binding VH and VL of alsevalimab or the VH and VL pairs shown in U.S. Pat. Nos. 10,626,176; 9,676,854; 9,574,000; 10,150,813; 10,814,011 or in PCT publication nos. WO2009/073533, WO2019/165077, WO2019/169212, WO2019/147670, WO2021/155307, WO2022/039490, WO2019/154315 or WO2021/185934.

In some embodiments, the tumor antigen potentiator module is a polypeptide selectively expressed or overexpressed on a tumor cell. In some embodiments, said polypeptide when inhibited, decreases the proliferation and/or the survival of a tumor cell.

In other embodiments, the tumor antigen binding domain of a CAR potentiator module can be replaced by a viral, bacterial or microbial binding domain (e.g., for use in the treatment of an infectious disease), a binding domain that binds a protein expressed by a pro-inflammatory cell, or a binding domain specific to any other antigen of interest expressed by a target cell (e.g. a target cell which is to be eliminated), for example where the target cell is an infected cell (e.g. virally infected), a bacterial cell, or a pro-inflammatory cell.

In some embodiments, a bacterial or microbial binding domain can bind a bacterial antigen. As used herein, the term bacterial antigen includes, but is not limited to, intact, attenuated or killed bacteria, any structural or functional bacterial protein or carbohydrate, or any peptide portion of a bacterial protein of sufficient length (typically about 8 amino acids or longer) to be antigenic. Examples include gram-positive bacterial antigens and gram-negative bacterial antigens. In some embodiments the bacterial antigen is derived from a bacterium selected from the group consisting of Helicobacter species, in particular Helicobacter pyloris; Borrelia species, in particular Borrelia burgdorferi; Legionella species, in particular Legionella pneumophilia; Mycobacterias species, in particular M. tuberculosis, M. avium, M. intracellulare, M. kansasii, M. gordonae; Staphylococcus species, in particular Staphylococcus aureus; Neisseria species, in particular N. gonorrhoeae, N. meningitidis; Listeria species, in particular Listeria monocytogenes; Streptococcus species, in particular S. pyogenes, S. agalactiae; S. faecalis; S. bovis, S. pneumoniae; anaerobic Streptococcus species; pathogenic Campylobacter species; Enterococcus species; Haemophilus species, in particular Haemophilus influenzae; Bacillus species, in particular Bacillus anthracis; Corynebacterium species, in particular Corynebacterium diphtheriae; Erysipelothrix species, in particular Erysipelothrix rhusiopathiae; Clostridium species, in particular C. perfringens, C. tetani; Enterobacter species, in particular Enterobacter aerogenes, Klebsiella species, in particular Klebsiella 1S pneumoniae, Pasteurella species, in particular Pasteurella multocida, Bacteroides species; Fusobacterium species, in particular Fusobacterium nucleatum; Streptobacillus species, in particular Streptobacillus moniliformis; Treponema species, in particular Treponema pertenue; Leptospira; pathogenic Escherichia species; and Actinomyces species, in particular Actinomyces israeli.

In some embodiments, a viral binding domain binds to a viral antigen. As used herein, the term viral antigen includes, but is not limited to, intact, attenuated or killed whole virus, any structural or functional viral protein, or any peptide portion of a viral protein of sufficient length (typically about 8 amino acids or longer) to be antigenic. Sources of a viral antigen include, but are not limited to viruses from the families: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., Ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviruses and rotaviruses); Bornaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), Hepatitis C; Norwalk and related viruses, and astroviruses). Alternatively, a viral antigen may be produced recombinantly.

### (3) Format of the Potentiator Module

A CAR potentiator module can take several forms such as single chain proteins or polypeptides, heterodimeric proteins, heterotrimer proteins and heterotetramer proteins, and that can each be produced according to a variety of formats. The potentiator module can include different domains positioned on different polypeptide chain that associate to form a multimeric protein. A wide range of protein formats can advantageously be constructed around immunoglobulin Fc domain dimers that are capable of binding to human FcRn polypeptide (neonatal Fc receptor), with or without additionally binding to CD16 or CD16A and optionally other Fcγ receptors, e.g., CD16B, CD32A, CD32B and/or CD64). A CAR potentiator module can have an intrinsic activity by itself when not bound to the CAR signaling module. In particular, the potentiator module may mediate NK cell recruitment at the tumor site in the absence of engineered cells expressing the CAR signaling module. The greatest potentiation of NK cell cytotoxicity can be obtained through use of Fc moieties that have substantial binding to the activating human CD16 receptor (CD16A) binding; such CD16 binding can be obtained through the use of suitable CH2 and/or CH3 domains, as further described herein. In some embodiments, an Fc moiety is derived from a human IgG1 isotype constant region. Use of modified CH3 domains also contributes to the possibility of use a wide range of heteromultimeric structures. A CAR potentiator module can for example comprise a first and a second polypeptide chain each comprising a human Fc domain monomer (i.e. a CH2-CH3 unit), optionally a Fc domain monomer comprising a CH3 domain capable of undergoing preferential CH3-CH3 hetero-dimerization, wherein the first and second chain associate via CH3-CH3 dimerization and the protein consequently comprises a Fc domain dimer. Binding moieties (e.g. tumor antigen and NKp46 binding moieties) can respectively be connected, optionally via a peptide linker, to the first and/or second polypeptide. In one example, a CAR potentiator module can for example comprise a first and a second polypeptide chain each comprising a variable domain fused to a human Fc domain monomer (i.e. a CH2-CH3 unit), optionally a Fc domain monomer comprising a CH3 domain capable of undergoing preferential CH3-CH3 hetero-dimerization, wherein the first and second chain associate via CH3-CH3 dimerization and the protein consequently comprises a Fc domain dimer. The variable domains of each chain can be part of the same or different antigen binding domain (e.g. tumor antigen binding moiety, ECD binding moiety, or other binding domains).

In some optional embodiments, the CAR potentiator module can further comprise an ABD that binds to a cytokine receptor, for example a cytokine receptor present on NK cells (e.g. a receptor for IL2, IL15, IL18, IL21 or IFN-α).

In some embodiments, CAR potentiator module can thus be conveniently constructed using VH and VL pairs arranged as scFv or Fab structures, together with CH1 domains, CL domain, Fc domains and cytokines, and domain linkers. Preferably, the proteins will use minimal non-natural sequences, e.g. minimal use of non-Ig linkers, optionally no more than 5, 4, 3, 2 or 1 domain linker(s) that is not an antibody-derived sequence, optionally wherein domain linker(s) are no more than 15, 10 or 5 amino acid residues in length.

In one embodiment, the CAR potentiator module is configured such that the ECD-binding moiety (e.g. NKp46 binding moiety), where present the Fc domain (or other CD16A-binding domain), and where present the cytokine receptor-binding domain, are each capable of binding to their respective NKp46, CD16A or cytokine receptor binding partner when such binding partners are present together at the surface of a cell (e.g. co-binding to an NK cell). In any embodiment, an ABD that binds to a human NKp46 polypeptide and an ABD that binds a human cytokine receptor can be specified as being configured to be capable of adopting a membrane planar binding confirmation. Optionally, the CAR potentiator module can be specified as being capable of interacting with, binding to or co-engaging NKp46 and the cytokine receptor, and optionally further CD16A, on the surface of an NK cell.

In some embodiments, the CAR potentiator module can be characterized by monovalent binding to NKp46 (e.g. the multispecific protein comprises only one NKp46 ABD), monovalent (or optionally bivalent) binding to antigen of interest (i.e. the protein has one or optionally two ABDs that bind a tumor antigen), monovalent binding to CD16A (e.g. the CAR potentiator module comprises only one Fc domain dimer or CD16A-binding ABD), and where a cytokine is present, monovalent binding to cytokine receptor (e.g., the CAR potentiator module comprises only one cytokine receptor ABD). In some embodiment, the ABD that binds to a human NKp46 polypeptide and the ABD that binds a human cytokine receptor, and optionally further the Fc domain, are specified as being positioned or connected within the multispecific protein in series (e.g. with respect to the N- and C-termini of the protein).

The ABD that binds NKp46 (e.g. a Fab, single variable domain or scFv) can optionally be specified as being connected to the CD16A-binding domain (e.g. an Fc domain) by an Ig-derived (e.g. a peptide from a hinge domain or heavy or light chain constant domain) or non-Ig-derived domain linker, optionally wherein the domain linker is a flexible polypeptide linker. The optional ABD that binds a cytokine receptor can optionally be specified as comprising a wild-type or variant cytokine connected to the rest of the CAR potentiator or to the NKp46 ABD by a domain linker, optionally a flexible polypeptide linker. The cytokine can be specified as being positioned C-terminal to both the NKp46- and CD16A-binding domains on the CAR potentiator, and optionally further the cytokine is connected to the rest of the CAR potentiator (or e.g., a domain thereof, the NKp46 ABD) via a peptide linker of 15, 10 or 5 residues or less. The NKp46- and CD16A-binding domains can optionally be specified as being placed adjacent to one another on the CAR potentiator protein and optionally connected to one another by a peptide linker (e.g. an immunoglobulin-derived linker such as a hinge-derived linker, a non-immunoglobulin-derived linker, a flexible linker) having a length of 15, 10 or 5 residues or less.

In some embodiments, the CAR potentiator module (e.g. dimers, trimers, tetramers) may comprise a domain arrangement of any of the following in which domains can be placed on any of the 2, 3 or 4 polypeptide chains, wherein the ECD binding moiety (e.g. a NKp46 ABD) is connected to one of the polypeptide chains of the Fc domain dimer via a hinge polypeptide or a flexible linker, wherein “n” is 1 or 2:
(Anti-tumor antigen ABD)n-(Fc domain dimer)-(ECD binding moiety)

In some embodiment, a CAR potentiator module (e.g. dimers, trimers, tetramers) may comprise a cytokine receptor antigen binding domain. In some embodiments, the CAR potentiator module may comprise a domain arrangement of any of the following in which domains can be placed on any of the 2, 3 or 4 polypeptide chains, wherein the ECD binding moiety (e.g. a NKp46 binding moiety) is interposed between the Fc domain and the cytokine receptor ABD (e.g. the protein has a terminal or distal cytokine receptor ABD at the C-terminal end and a terminal or distal tumor antigen of interest ABD at the topological N-terminal end), wherein the ECD binding moiety (e.g. a NKp46 binding moiety) is connected to one of the polypeptide chains of the Fc domain dimer via a hinge polypeptide or a flexible linker, and wherein the ABD that binds the cytokine receptor is connected to the ECD binding moiety (e.g. a NKp46 binding moiety, e.g. to one of the polypeptide chains thereof when the NKp46 binding moiety is contained on two chains) via a flexible linker (e.g. a linker comprising G and S residues), wherein “n” is 1 or 2:
(Anti-tumor antigen ABD)n-(Fc domain dimer)-(ECD binding moiety)-(cytokine receptor ABD).

In some embodiments, the cytokine receptor ABD can be an IL2, IL15, IL18, IL21 or IFN-α polypeptide. In some embodiments, the Fc domain can be specified to be a Fc domain dimer (e.g. that binds human FcRn and/or Fcγ receptors). In one embodiment, one or both of the antigen of interest (e.g. cancer antigen) ABD and NKp46 ABD is formed from two variable regions present within tandem variable regions, wherein the variable regions that associate to form a particular ABD can be on the same polypeptide chain or on different polypeptide chains. In another embodiment, one or both of the antigen of interest (tumor antigen) ABD and ECD binding moiety (e.g. NKp46 ABD) comprises a tandem variable region and the other comprises a Fab structure. In another embodiment, both of the antigen of interest and the ECD binding moiety (e.g. NKp46 ABD) comprises a Fab structure. In other embodiments one of the antigen of interest and ECD binding moiety (e.g. NKp46 ABD) comprises a Fab structure and the other comprises an scFv structure.

According to some embodiments, heterodimeric or heterotrimeric polypeptides have an NKp46 binding ABD as ECD binding moiety, a tumor antigen binding ABD, optionally a cytokine receptor-binding ABD (e.g. IL-2, IL-15, IL-21, IL-7, IL-27, IL-12, IL-18, IFN-α or IFN-β polypeptide) and a Fc domain dimer can optionally be produced as one or more chains that each associate with a central chain, e.g. by CH1-Ck heterodimerization and/or by CH3-CH3 dimerization. Different variants can be produced, as illustrated in the Examples herein.

In some embodiments, the cytokine is a modified IL-2 (IL2v) which has decreased or abolished binding to CD25 (expressed inter alia on regulatory T cells) compared to wild-type IL-2, but retains at least partial binding to human CD122 (or to a CD122:CD132 complex). The modified IL-2 comprise can optionally be specified as exhibiting a KD for binding to CD25 or to a CD25:CD122:CD132 complex that is decreased by at least 1-log, optionally at least 2-log, optionally at least 3-log, compared to a wild-type human IL-2 polypeptide. A modified IL-2 can optionally be specified as exhibiting less than 20%, 30%, 40% or 50% of binding affinity to CD25 or to a CD25:CD122:CD132 complex compared to a wild-type human IL-2 polypeptide. An IL2 can optionally be specified as exhibiting at least 50%, 70%, 80% or 90% of binding affinity to CD122 or to a CD122:CD132 complex compared to a wild-type human IL-2 polypeptide. In some embodiments, an IL-2 exhibits at least 50%, 60%, 70% or 80% but less than 100% of binding affinity to CD122 or to a CD122:CD132 complex compared to a wild-type human IL-2 polypeptide. In some embodiments, an IL2v exhibits less than 50% of binding affinity to CD25 and at least 50%, 60%, 70% or 80% of binding affinity to CD122, compared to wild-type IL-2 polypeptide.

Differences in binding affinity of wild-type and disclosed mutant polypeptide for CD25 and CD122 and complexes thereof can be measured, e.g., in standard surface plasmon resonance (SPR) assays that measure affinity of protein-protein interactions familiar to those skilled in the art.

Exemplary IL2 variant polypeptides have one or more, two or more, or three or more CD25-affinity-reducing amino acid substitutions relative to the wild-type mature IL-2 polypeptide having an amino acid sequence of SEQ ID NO: 404. In one embodiment, the exemplary IL2v polypeptides comprise one or more, two or more, or three or more substituted residues selected from the following group: Q11, H16, L18, L19, D20, D84, S87, Q22, R38, T41, F42, K43, Y45, E62, P65, E68, V69, L72, D84, S87, N88, V91, I92, T123, Q126, S127, I129, and S130.

In one embodiment, the exemplary IL2 variant polypeptide has one, two, three, four, five or more of amino acid residues position R38, T41, F42, F44, E62, P65, E68, Y107, or S125 substituted with another amino acid.

In one embodiment, decreased affinity to CD25 or a protein complex comprising such (e.g., a CD25:CD122:CD132 complex) may be obtained by substituting one or more of the following residues in the sequence of the wild-type mature IL-2 polypeptide: R38, F42, K43, Y45, E62, P65, E68, V69, and L72.

In one embodiment, an isolated or purified heterodimeric or heterotrimeric CAR potentiator module comprises at least two or three polypeptide chains, each comprising a V-(CH1/Ck) unit, whereby the chains are bound to one another by non-covalent interactions and optionally further bound via disulfide bonds between CH1 and Ck domains, and still further optionally, whereby the chains are bound by non-covalent interactions between the respective variable regions, CH1 and Ck domains, and CH3 domains of the Fc portion.

In one embodiment, the CAR potentiator module comprises a first and a second polypeptide chain each comprising a variable domain fused to a CH1 or Ck domain (a V-(CH1/Ck) unit), in turn fused at its C-terminus to a human Fc domain monomer comprising a CH2 domain and a CH3 domain capable of undergoing CH3-CH3 dimerization, wherein the first and second chain associate via CH1-Ck and CH3-CH3 dimerization such that the protein comprises a Fc domain dimer. The variable domains of each chain can be part of the same or different antigen binding domains.

The variable and constant regions can be selected and configured such that each chain will preferentially associate with its desired complementary partner chain. The resulting multimeric CAR potentiator module can be produced reliably and with high productivity using conventional production methods using recombinant host cells. The choice of which VH or VL to associate with a CH1 and Ck in a unit is based on affinity between the units to be paired so as to drive the formation of the desired multimer. The resulting multimer will be bound by non-covalent interactions between complementary VH and VL domains, by non-covalent interactions between complementary CH1 and Ck domains, and optionally by further disulfide bonding between complementary CH1 and Ck domains (and optionally further disulfide bonds between complementary hinge domains). VH-VL associations are stronger than VH-VH or VL-VL, consequently, as shown herein, one can place a VH or a VL next to either a CH1 or a Ck, and the resulting V-C unit will partner preferably with its V-C counterpart. For example VH-Ck will pair with VL-CH1 preferentially over VH-CH1. Additionally, by including an Fc domain, preferred chain pairing is further improved, as the two Fc monomer-containing chains are bound by non-covalent interactions between CH3 domains of the Fc domain monomers.

In one embodiment, the CAR potentiator module is a heterodimer comprising a first and a second polypeptide chain each comprising a variable domain fused to a CH1 or Ck domain (a V-(CH1/Ck) unit), in turn fused at its C-terminus to a human Fc domain monomer, wherein the V-(CH1/Ck) unit of the first chain has undergone CH1-Ck dimerization with the V-(CH1/Ck) unit of the second chain thereby forming a first antigen binding domain (ABD1) and a Fc domain dimer, wherein one of the polypeptide chains further comprises an antigen binding domain that forms a second antigen binding domain (ABD2), and wherein the Fc domain dimer binds to a human CD16 polypeptide, wherein one of ABD1 and ABD2 is a ECD binding domain (e.g. binds NKp46) and the other binds the antigen of interest (e.g. tumor antigen).

In one example, the protein has a domain arrangement as shown below, where “Cyt” is optional (can be present or absent) and indicates a cytokine:

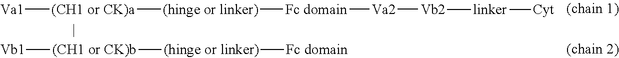

wherein Va-1, Vb-1, Va-2 and Vb-2 are each a VH domain or a VL domain, and wherein one of Va-1 and Vb-1 is a VH and the other is a VL such that Va-1 and Vb-1 form a first antigen binding domain (ABD), wherein one of Va-2 and Vb-2 is a VH and the other is a VL such that Va-2 and Vb-2 form a second antigen binding domain, wherein one of the ABD is a ECD binding domain (e.g. binds NKp46) and the other binds an antigen of interest.

In other embodiments, the protein is a heterotrimer and comprises three polypeptide chains, each comprising a variable domain fused to a CH1 or Ck domain (a V-(CH1/Ck) unit), wherein a first (central) chain comprises two V-(CH1/Ck) units and a human Fc domain interposed between the units, the second chain comprises one V-(CH1/Ck) unit and a human Fc domain monomer, and the third chain comprises one V-(CH1/Ck) unit and optionally a cytokine polypeptide (Cyt), wherein one of the V-(CH1/Ck) units of the central chain has undergone CH1-Ck dimerization with the V-(CH1/Ck) unit of the second chain thereby forming a first antigen binding domain (ABD1) and a Fc domain dimer, and wherein the other of the V-(CH1/Ck) units of the central chain has undergone CH1-Ck dimerization with the V-(CH1/Ck) unit of the third chain thereby forming a second antigen binding domain (ABD2), and wherein the Fc domain binds to a human CD16 polypeptide. In one embodiment, the Fc domain comprises N-linked glycosylation at residue N297 (Kabat EU numbering).

In one embodiment, the binding protein comprises a first, second and third polypeptide chains (I), (II) and (Ill) that comprise two ABDs and has a domain arrangement:

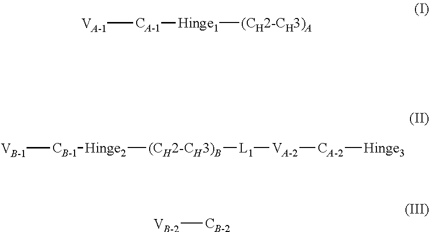

wherein:
V_{A-1} and V_{B-1} form a binding pair V₁ (V_{H1}/V_{L1});
V_{A-2} and V_{B-2} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein CH1 is an immunoglobulin heavy chain constant domain 1 and C_{L} is an immunoglobulin light chain constant domain;
Hinge₁, Hinge₂ and optional Hinge₃ are identical or different and correspond to all or part of an immunoglobulin hinge region;
(C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
L₁ is an amino acid linker. In one embodiment, binding pair V₁ binds to an antigen of interest (e.g. tumor antigen) and binding pair V₂ is an ECD binding domain (e.g., V₂ binds to NKp46).

In one example, the protein has a domain arrangement:

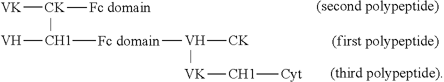

In another embodiment, the protein has a domain arrangement:

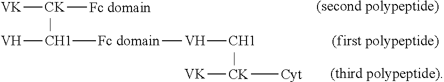

In the above heterotrimeric proteins, the cytokine (Cyt) is optional and can be present or absent. The Fab structure interposed between the Fc domain and the cytokine is the ECD binding moiety (e.g. NKp46 binding ABD) (i.e. the ECD binding moiety is interposed between the Fc domain and the C-terminal cytokine). The Fc domain in the first polypeptide is connected (e.g. fused) at its C terminus to the N-terminus of the VH domain via a linker. The constant domain (the CH1 or CK domain in the respective domain arrangements) in the third polypeptide is connected (e.g. fused) at its C terminus to the N-terminus of the cytokine polypeptide via a linker. Each constant domain (the CH1 or CK domain in the respective domain arrangements) that is N-terminal to the Fc domain is fused at the C terminus of the constant domain to the N-terminus of the Fc domain via a hinge region.

Optionally, any of the CAR potentiator modules may include CH1, CL or CH3 domains which comprise amino acid modifications (e.g. substitutions) to promote heterodimerization. For example, heterodimerization modifications often involve steric repulsion, charge steering interaction, or interchain disulfide bond formation, wherein the CH3 domain interface of the antibody Fc region is mutated to create altered charge polarity across the Fc dimer interface such that co-expression of electrostatically matched Fc chains supports favorable attractive interactions thereby promoting desired Fc heterodimer formation, whereas unfavorable repulsive charge interactions suppress unwanted Fc homodimer formation.

In one embodiment, the first (central) polypeptide chain will provide one variable domain that will, together with a complementary variable domain on a second polypeptide chain, form a first antigen binding domain (e.g. the ABD that binds the antigen of interest), and an Fc domain. The first (central) polypeptide chain will also provide a second variable domain (e.g., placed on the opposite end of the interposed Fc domain from the first variable domain, at the C-terminus of the Fc domain) that will be paired with a complementary variable domain to form a second antigen binding domain (e.g. the switching binding domain, that can be an ABD that binds NKp46); the variable domain that is complementary to the second variable domain can be placed on the central polypeptide (e.g. adjacent to the second variable domain in a tandem variable domain construct such as an scFv), or can be placed on a separate polypeptide chain, notably a third polypeptide chain. The second (and third, if present) polypeptide chains will associate with the central polypeptide chain by CH1-Ck heterodimerization, forming non-covalent interactions and optionally further interchain disulfide bonds between complementary CH1 and Ck domains (and optionally interchain disulfide bonds between hinge regions), with a primary multimeric polypeptide being formed so long as CH/Ck and VH/Vk domains are chosen to give rise to a preferred dimerization configuration that results preferentially in the desired VH-VL pairings. Remaining unwanted pairings can remain minimal during production and/or are removed during purification steps. In a trimer, or when polypeptides are constructed for preparation of a trimer, there will generally be one polypeptide chain that comprises a non-naturally occurring VH-Ck or Vk-CH1 domain arrangement. Optionally, a cytokine (e.g., IL-2, IL-15, IL-21, IL-7, IL-27, IL-12, IL-18, IFN-α or IFN-β) can then be placed at the C-terminus of one of polypeptide chains. The cytokine can be fused via a domain linker, and while not shown in certain domain arrangements herein, any domain arrangement can be specified as comprising a domain linker separating two domains. For example, in these structures, the cytokine can be placed at the C-terminus of the first (central) polypeptide chain or at the C-terminus of the third polypeptide chain (when such third chain is present).

In some embodiments, when the CAR potentiator module is a heterodimer, the antigen binding domain for the second antigen of interest (i.e. the ECD binding domain can then be formed from Va-2 and Vb-2 which are configured as tandem variable domains on the central chain forming an ABD (e.g. forming an scFv unit).

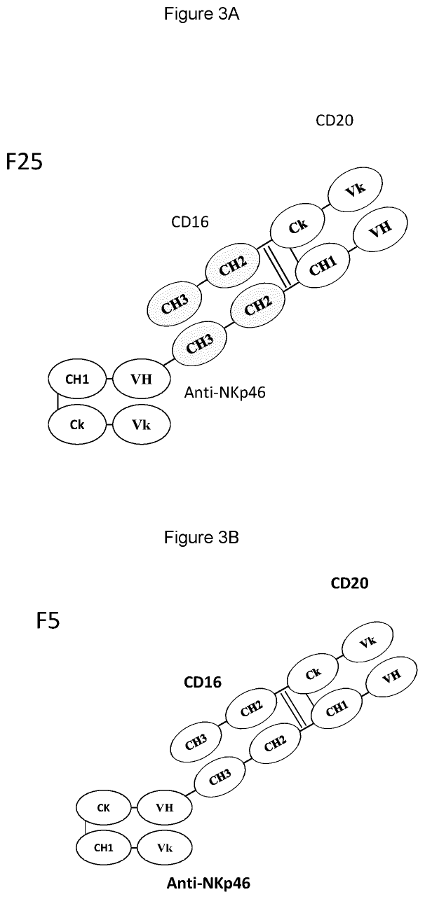

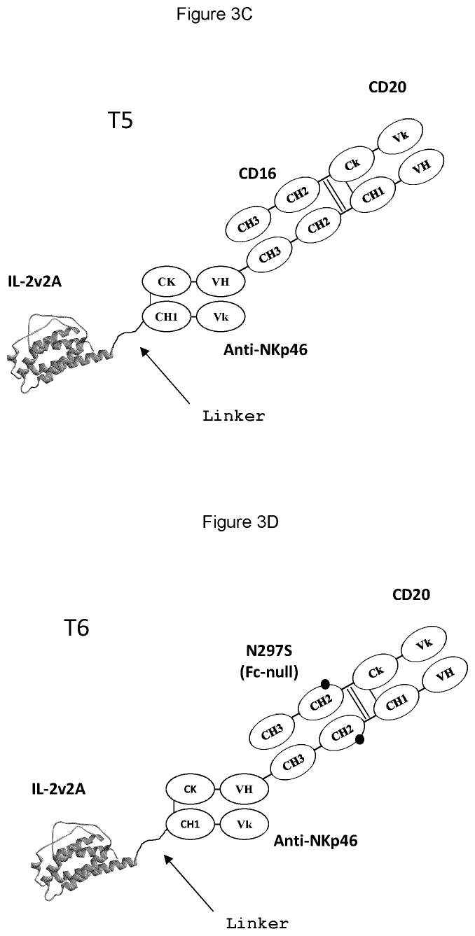

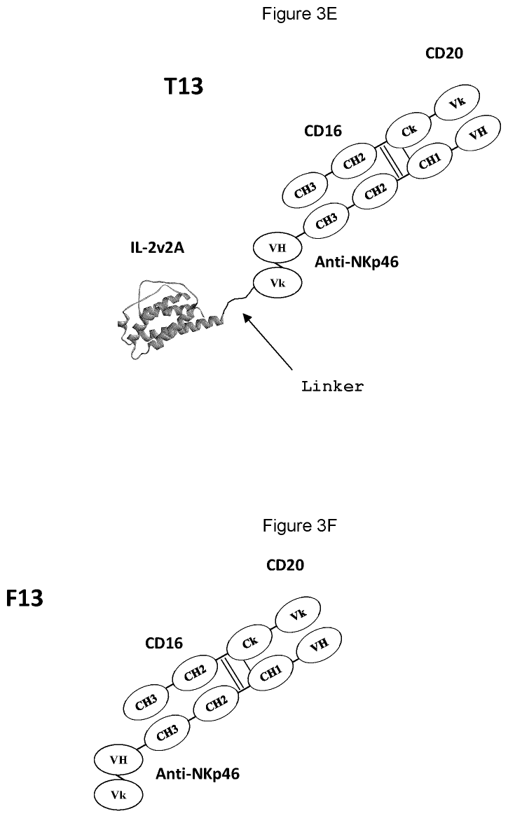

The resulting heterodimer can, for example, have the following configuration (see also example of such protein shown as format T13 in FIG. 3E).

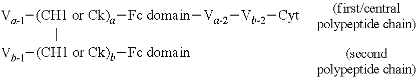

wherein one of Va-1 of the first polypeptide chain and Vb-1 of the second polypeptide chain is a light chain variable domain and the other is a heavy chain variable domain, and wherein one of Va-2 and Vb-2 is a light chain variable domain and the other is a heavy chain variable domain. Va-2 and Vb-2 can be specified as being separated by a polypeptide linker (Va-2 and Vb-2 form an scFv). Va-2 and Vb-2 forms the ABD that binds NKp46 and Va-1 and Vb-1 forms the ABD that binds the antigen of interest (e.g. cancer antigen). The cytokine (Cyt) is optional and can be present or absent.

In some embodiments, when the CAR potentiator module is a heterotrimer wherein the antigen binding domain for the second antigen of interest (i.e. the ECD binding domain, e.g. a NKp46 binding domain) is interposed between the Fc domain and the optional cytokine polypeptide, it can be formed by using a central (first) polypeptide chain comprising a first variable domain (V) fused to a first CH1 or CK constant region, a second variable domain (V) fused to a second CH1 or CK constant region, and an Fc domain or portion thereof interposed between the first and second variable domains (i.e. the Fc domain is interposed between the first and second (V-(CH1/Cκ) units. For example, a central polypeptide chain for use in a heterotrimeric protein can have the domain arrangements (N- to C-termini) as follows:

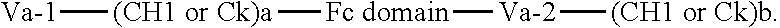

The first polypeptide chain can optionally further have a Cyt is placed at its C-terminus.

A second polypeptide chain can then comprise a domain arrangement (N- to C-termini from left to right):

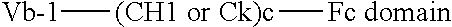

such that the (CH1 or Ck)c dimerizes with the (CH1 or Ck)a on the central chain, and the Va-1 and Vb-1 form an antigen binding domain that binds the antigen of interest.

A third polypeptide chain can then comprise the following domain arrangement (N- to C-termini from left to right):

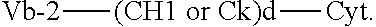

such that the (CH1 or Cκ)d dimerizes with the (CH1 or Cκ)b unit on the central chain, and the Va-2 and Vb-2 form the NKp46 binding domain.

Optionally, when the optional Cyt is placed at the C-terminus of the first polypeptide chain, the third polypeptide chain can then comprise the following domain arrangement (N- to C-termini from left to right):

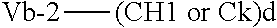

The resulting heterotrimers can, for example, have the following configuration (See also example of such protein shown as formats T5 or T6 in FIGS. 3C and 3D).

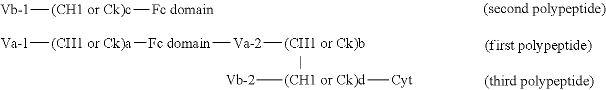

A domain configuration of a resulting heterotrimer where the Cyt is placed on the first polypeptide chain is shown below:

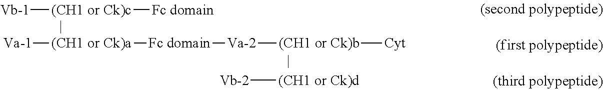

In the above heterotrimeric proteins, the cytokine (Cyt) is optional and can be present or absent. In a trimeric polypeptide in which the ECD binding moiety (e.g. a NKp46 ABD) is interposed between the Fc domain and the cytokine polypeptide, the first polypeptide can have two variable domains that each form an antigen binding domain with a variable domain on a separate polypeptide chain (i.e. the variable domain of the second and third chains), the second polypeptide chain has one variable domain, and the third polypeptide has one variable domain, and one of the polypeptide chains comprises a cytokine polypeptide fused to its C-terminus.

In one embodiment, a heterotetramer comprises a polypeptide chain 1, 2, 3 and 4:

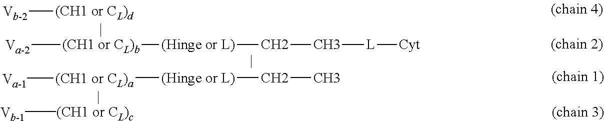

wherein:
Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD) that binds an antigen of interest, wherein one of Vₐ₋₂ and V_{b-2} is a V_{H} and the other is a V_{L} and wherein Vₐ₋₂ and V_{b-2} form a second ABD that binds NKp46;
CH1 is a human immunoglobulin CH1 domain and CL is a light chain constant domain;
one of (CH1 or CL)ₐ and (CH1 or CL)_{c} is a CH1 and the other is a CL such that a (CH1/CL) pair is formed;
one of (CH1 or CL)_{b} and (CH1 or CL)_{d} is a CH1 and the other is a CL such that a (CH1/CL) pair is formed;
Hinge is an immunoglobulin hinge region or portion thereof;
L is an amino acid domain linker, wherein each L can be different or the same;
CH2 and CH3 are human immunoglobulin CH2 and CH3 domains, respectively; and
Cyt, optional, can be absent (together with the L that connects it to CH3) or is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

In one embodiment, provided is a heterotrimer having a polypeptide chain 1, 2 and 3:

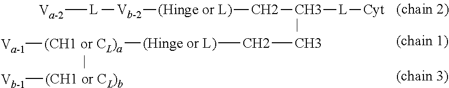

wherein:
Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD) that binds an antigen of interest, wherein one of Vₐ₋₂ and V_{b-2} is a V_{H} and the other is a V_{L} and wherein Vₐ₋₂ and V_{b-2} form a second ABD that binds NKp46;
CH1 is a human immunoglobulin CH1 domain and CL is a light chain constant domain;
one of (CH1 or CL)ₐ and (CH1 or CL)_{b} is a CH1 and the other is a CL such that a (CH1/CL) pair is formed;
Hinge is an immunoglobulin hinge region or portion thereof;
L is an amino acid domain linker, wherein each L can be different or the same;
CH2 and CH3 are human immunoglobulin CH2 and CH3 domains, respectively; and
Cyt, optional, can be absent (together with the L that connects it to CH3) or is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

In any embodiment herein, it may be specified that the protein has a Fc domain dimer comprised of a first and second Fc domain monomer placed on separate chains that dimerize via CH3-CH3 association, wherein one of the Fc domain monomers is connected to the both the ECD binding domain (e.g. anti-NKp46 ABD) and the optional cytokine, and the other (second) Fc domain monomer has a free C-terminus (no ECD binding domain or cytokine fused to its C-terminus).

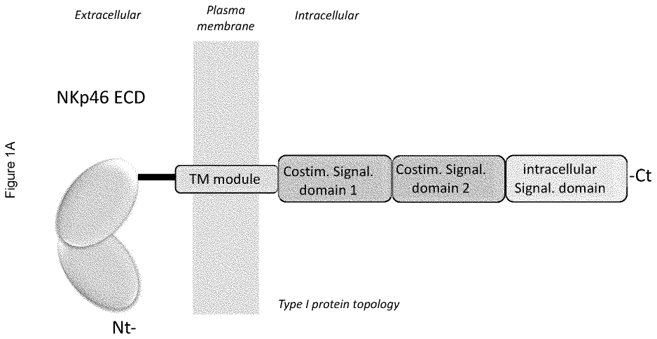

Optionally in any embodiment herein, fusions or linkages on the same polypeptide chain between different domains (e.g., between two V domains placed in tandem, between V domains and CH1 or Ck domains, between CH1 or Ck domains and Fc domains, between Fc domains monomers and V domains, between Fc domain monomers and the optional cytokine) may occur via intervening amino acid sequences, for example via a hinge region or linker peptide. Generally, domain arrangements or structures herein are depicted without showing domain linkers, and it will be appreciated that the domain arrangements can be specified as having domain linkers between a specified domain. For example, the cytokine, when present, can be specified as being fused to an adjacent domain via a domain linker, and a domain linker can be inserted in the relevant domain arrangement or structure. In another example, tandem variable domains (e.g. in an scFv) can be specified as being fused to one another via a domain linker, and a domain linker can be inserted between the two V regions in the relevant domain arrangement or structure. In another example, a CH1 or CL (or Ck) constant region can be fused to an Fc domain or CH2 domain thereof via a domain linker or hinge domain or portion thereof, and accordingly a domain linker or hinge domain or portion thereof can be inserted between CH1 or CL domain and the Fc domain or CH2 domain in the relevant domain arrangement or structure. An example of the domain arrangement of a multispecific protein with linkers shown is shown in FIG. 1B for the representative heterotrimer in format “T5”, shows domain linkers such as hinge and glycine-serine linkers, and interchain disulfide bridges.

In any embodiment herein, a polypeptide chain (e.g., chain 1, 2, 3 or 4) can be specified as having a free N and/or C terminus (no other protein domains at the terminus of the polypeptide chain).

In any embodiment herein, the proteins domains described herein can optionally be specified as being indicated from N- to C-termini. Protein arrangements of the disclosure for purposes of illustration are shown from N-terminus (on the left) to C-terminus (on the right). Adjacent domains on a polypeptide chain can be referred to as being fused to one another (e.g. a domain can be said to be fused to the C-terminus of the domain on its left, and/or a domain can be said to be fused to the N-terminus of the domain on its right). The proteins domains described herein can be fused to one another directly (e.g. V domains fused directly to CH1 or CL domains) or via linkers or short intervening amino acid sequences that serve to connect the domains on a polypeptide chain (e.g. they may optionally be specified to lack other predetermined functionality, or to lack specific binding to a predetermined ligand). Two polypeptide chains will be bound to one another (indicated by “|”), by non-covalent interactions, and optionally can further be attached via interchain disulfide bonds, formed between cysteine residues within complementary CH1 and C domains.

In some embodiments, a CAR potentiator module can include a large variety of linkers (e.g. a glycine-serine linkers) suitable to connect adjacent protein domains. Such linkers are described in above part (iii) as well as hereinafter. Adjacent protein domains can be specified as being connected or fused to one another by a domain linker. An exemplary domain linker is a (poly)peptide linker, optionally a flexible (poly)peptide linker. Peptide linkers or polypeptide linkers, used interchangeably herein, may have a subsequence derived from a particular domain such as a hinge, CH1 or CL domain, or may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 2 to 30 amino acids in length. In one embodiment, linkers of 4 to 20 amino acids in length may be used, with from about 5 to about 15 amino acids finding use in some embodiments. While any suitable linker can be used, many embodiments, linkers (e.g. flexible linkers) can utilize a glycine-serine polypeptide or polymer, including for example comprising (GS)n, (GSGGS)n, (GGGGS)n, (GSSS)n, (GSSSS)n and (GGGS)n, where n is an integer of at least one (optionally n is 1, 2, 3 or 4), glycine-alanine polypeptide, alanine-serine polypeptide, and other flexible linkers. Linkers comprising glycine and serine residues generally provides protease resistance. One example of a (GS)1 linker is a linker having the amino acid sequence STGS; such a linker can be useful to fuse a domain to the C-terminus of an Fc domain (or a CH3 domain thereof). In some embodiments peptide linkers comprising (G2S)n are used, wherein, for example, n=1-20, e.g., (G2S), (G2S)2, (G2S)3, (G2S)4, (G2S)5, (G2S)6, (G2S)7 or (G2S)8, or (G3S)n, wherein, for example, n is an integer from 1-15. In one embodiment, a domain linker comprises a (G4S)n peptide, wherein, for example, n is an integer from 1-10, optionally 1-6, optionally 1-4. In some embodiments peptide linkers comprising (GS2)n (GS3)n or (GS4)n are used, wherein, for example, n=1-20, e.g., (GS2), (GS2)2, (GS2)3, (GS3)1, (GS3)2, (GS3)3, (GS4)1, (GS4)2, (GS4)3, wherein, for example, n is an integer from 1-15. In one embodiment, a domain linker comprises a (GS4)n peptide, wherein, for example, n is an integer from 1-10, optionally 1-6, optionally 1-4. In one embodiment, a domain linker comprises a C-terminal GS dipeptide, e.g., the linker comprises (GS4) and has the amino acid sequence a GSSSS (SEQ ID NO: 25), GSSSSGSSSS (SEQ ID NO: 26), GSSSSGSSSSGS (SEQ ID NO: 27) or GSSSSGSSSSGSSSS (SEQ ID NO: 28).

Any of the (poly)peptide or domains linkers described herein may be specified to comprise a length of at least 2 residues, 3 residues, 4 residues, at least 5 residues, at least 10 residues, or more. In other embodiments, the linkers comprise a length of between 2-4 residues, between 2-6 residues, between 2-8 residues, between 2-10 residues, between 2-12 residues, between 2-14 residues, between 2-15 residues, between 2-16 residues, between 2-18 residues, between 2-20 residues, between 2-22 residues, between 2-24 residues, between 2-26 residues, between 2-28 residues, between 2-30 residues, between 2-50 residues, or between 10-50 residues.

Examples of polypeptide linkers may include sequence fragments from CH1 or CL domains; for example the first 4-12 or 5-12 amino acid residues of the CL/CH1 domains are particularly useful for use in linkages of scFv moieties. Linkers can be derived from immunoglobulin light chains, for example Ck or Cλ. Linkers can be derived from immunoglobulin heavy chains of any isotype, including for example Cy1, Cy2, Cy3, Cy4 and Cμ. Linker sequences may also be derived from other proteins such as Ig-like proteins (e.g. TCR, FcR, KIR), hinge region-derived sequences, and other natural sequences from other proteins. In certain domain arrangements, VH and VL domains are linked to another in tandem separated by a linker peptide (e.g. a scFv) and in turn be fused to the N- or C-terminus of an Fc domain (or CH2 domain thereof). Such tandem variable regions or scFv can be connected to the Fc domain via a hinge region or a portion thereof, an N-terminal fragment of a CH1 or CL domain, or a glycine- and serine-containing flexible polypeptide linker.

Fc domains can be connected to other domains via immunoglobulin-derived sequence or via non-immunoglobulin sequences, including any suitable linking amino acid sequence. Advantageously, immunoglobulin-derived sequences can be readily used between CH1 or CL domains and Fc domains, in particular, where a CH1 or CL domain is fused at its C-terminus to the N-terminus of an Fc domain (or CH2 domain). An immunoglobulin hinge region or portion of a hinge region can and generally will be present on a polypeptide chain between a CH1 domain and a CH2 domain. A hinge or portion thereof can also be placed on a polypeptide chain between a CL (e.g. Ck) domain and the CH2 domain of an Fc domain when a CL is adjacent to an Fc domain on the polypeptide chain. However, it will be appreciated that a hinge region can optionally be replaced for example by a suitable linker peptide, e.g. a flexible polypeptide linker.

Examples of polypeptide linkers may include sequence fragments from CH1 or CL domains; for example the first 4-12 or 5-12 amino acid residues of the CL/CH1 domains are particularly useful for use in linkages of scFv moieties. Linkers can be derived from immunoglobulin light chains, for example Ck or Cλ. Linkers can be derived from immunoglobulin heavy chains of any isotype, including for example Cy1, Cy2, Cy3, Cy4 and Cμ. Linker sequences may also be derived from other proteins such as Ig-like proteins (e.g. TCR, FcR, KIR), hinge region-derived sequences, and other natural sequences from other proteins. In certain domain arrangements, VH and VL domains are linked to another in tandem separated by a linker peptide (e.g. a scFv) and in turn be fused to the N- or C-terminus of an Fc domain (or CH2 domain thereof). Such tandem variable regions or scFv can be connected to the Fc domain via a hinge region or a portion thereof, an N-terminal fragment of a CH1 or CL domain, or a glycine- and serine-containing flexible polypeptide linker.

Fc domains can be connected to other domains via immunoglobulin-derived sequence or via non-immunoglobulin sequences, including any suitable linking amino acid sequence. Advantageously, immunoglobulin-derived sequences can be readily used between CH1 or CL domains and Fc domains, in particular, where a CH1 or CL domain is fused at its C-terminus to the N-terminus of an Fc domain (or CH2 domain). An immunoglobulin hinge region or portion of a hinge region can and generally will be present on a polypeptide chain between a CH1 domain and a CH2 domain. A hinge or portion thereof can also be placed on a polypeptide chain between a CL (e.g. Ck) domain and the CH2 domain of an Fc domain when a CL is adjacent to an Fc domain on the polypeptide chain. However, it will be appreciated that a hinge region can optionally be replaced for example by a suitable linker peptide, e.g. a flexible polypeptide linker.

The ECD binding moiety and the cytokine receptor ABD (when present) are linked to the rest of the CAR potentiator module (e.g. or to a constant region or Fc domain thereof) through a flexible linker (e.g. a polypeptide linker) that leads to less structural rigidity or stiffness (e.g. between or amongst the binding moiety and Fc domain) compared to a conventional (e.g. wild-type full length human IgG) antibody. For example, the CAR potentiator module may have a structure or a flexible linker between the ECD binding moiety and constant domain or Fc domain that permits an increased range or domain motion compared to the two binding moiety in a conventional (e.g. wild-type full length human IgG) antibody. In particular, the structure or a flexible linker can be configured to confer on the antigen binding sites greater intrachain domain movement compared to antigen binding sites in a conventional human IgG1 antibody. Rigidity or domain motion/interchain domain movement can be determined, e.g., by computer modeling, electron microscopy, spectroscopy such as Nuclear Magnetic Resonance (NMR), X-ray crystallography, or Sedimentation Velocity Analytical ultracentrifugation (AUC) to measure or compare the radius of gyration of proteins comprising the linker or hinge. A test protein or linker may have lower rigidity relative to a comparator protein if the test protein has a value obtained from one of the tests described in the previous sentence differs from the value of the comparator, e.g., an IgG1 antibody or a hinge, by at least 5%, 10%, 25%, 50%, 75%, or 100%. A cytokine can for example be fused to the C-terminus of a CH3 domain by a linker selected from GSSSS (SEQ ID NO: 25), GSSSSGSSSS (SEQ ID NO: 26), GSSSSGSSSSGS (SEQ ID NO: 27) or GSSSSGSSSSGSSSS (SEQ ID NO: 28).

In some embodiments, the C-terminal end of a CH1 or Ck domain (e.g. of a Fab) is connected to the N-terminal end of a CH2 domain by a hinge region, that can be a fragment of a hinge region (e.g. a truncated hinge region without cysteine residues) or may comprise one or more amino acid modifications which remove (e.g. substitute by another amino acid, or delete) a cysteine residue, optionally both cysteine residue in a hinge region. Removing cysteines can be useful to prevent undesired disulfide bond formation, e.g., the formation of disulfide bridges in a monomeric polypeptide.

A “hinge” or “hinge region” or “antibody hinge region” herein refers to the flexible polypeptide or linker between the first and second constant domains of an antibody. Structurally, the IgG CH1 domain ends at EU position 220, and the IgG CH2 domain begins at residue EU position 237. Thus for an IgG the hinge generally includes positions 221 (D221 in IgG1) to 236 (G236 in IgG1), wherein the numbering is according to the EU index as in Kabat. References to specific amino acid residues within constant region domains found within the polypeptides shall be, unless otherwise indicated or as otherwise dictated by context, be defined according to Kabat, in the context of an IgG antibody.

In some embodiments, the hinge region (or fragment thereof) is derived form a hinge domain of a human IgG1 antibody. For example a hinge domain may comprise the amino acid sequence: THTCPPCPAPELL (SEQ ID NO: 190) or a fragment comprising the first 8 resides thereof, or an amino acid sequence at least 60%, 70%, 80% or 90% identical to any of the foregoing, optionally wherein one or both cysteines are deleted or substituted by a different amino acid residue, optionally a serine.

In some embodiments, the hinge region (or fragment thereof) is derived from a Cμ2-C Cμ3 hinge domain of a human IgM antibody. For example a hinge domain may comprise the amino acid sequence: NASSMCVPSPAPELL (SEQ ID NO: 191), or an amino acid sequence at least 60%, 70%, 80% or 90% identical thereto, optionally wherein one or both cysteines are deleted or substituted by a different amino acid residue.

### Production of CAR Signaling and CAR Potentiator Modules

CAR modules can be produced by any means known in the art. In some embodiments, a CAR module is produced using recombinant DNA techniques. Nucleic acid sequences encoding each of the regions of the chain(s) of the CAR signaling modules and the CAR module can be prepared and assembled into complete coding sequences by standard techniques of molecular cloning. The resulting coding region can be inserted into an expression vector and used to transform a suitable expression cell line.

Polynucleotide gene sequences encoding more than one portion of an expressed CAR can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

In some embodiments, for expression of CAR signaling modules, an exogenous transcriptional initiation region can be used that allows for constitutive or inducible expression, wherein expression can be controlled depending upon the target host, the level of expression desired, the nature of the target host, and the like. CAR signaling modules are dedicated to be bound or anchored to the membrane of the effector cell (e.g., a T cell, a NK cell).

Likewise, a signal sequence directing the CAR signaling module to the surface membrane can be an endogenous signal sequence of the N-terminal component of the CAR. Optionally, in some instances, it may be desirable to exchange this sequence for a different signal sequence. However, the signal sequence selected should be compatible with the secretory pathway of the CAR expressing cells so that the CAR signaling module is presented on the surface of the CAR expressing cell.

Similarly, a termination region may be provided by the naturally occurring or endogenous transcriptional termination region of the nucleic acid sequence encoding the C-terminal component of the CAR module. Alternatively, the termination region may be derived from a different source. For the most part, the source of the termination region is generally not considered to be critical to the expression of a recombinant protein and a wide variety of termination regions can be employed without adversely affecting expression.

As will be appreciated by one of skill in the art, in some instances, a few amino acids at the ends of the binding domain in the CAR can be deleted, usually not more than 10, more usually not more than 5 residues, for example. Also, it may be desirable to introduce a small number of amino acids at the borders, usually not more than 10, more usually not more than 5 residues. The deletion or insertion of amino acids may be as a result of the needs of the construction, providing for convenient restriction sites, ease of manipulation, improvement in levels of expression, or the like. In addition, the substitute of one or more amino acids with a different amino acid can occur for similar reasons.

In some embodiments, CAR potentiator modules are produced by standard techniques.

Once appropriate antigen binding domains of the CAR potentiator modules having desired specificity and/or activity are identified, nucleic acids encoding each of the or binding domain can be separately placed, in suitable arrangements, in an appropriate expression vector or set of vectors, together with DNA encoding any elements such as CH1, Ck, CH2 and CH3 domains or portions thereof, optional mutant IL2 polypeptides and any other optional elements (e.g. DNA encoding a hinge-derived or linker elements) for transfection into an appropriate host. Binding domains will be arranged in an expression vector, or in separate vectors as a function of which type of polypeptide is to be produced, so as to produce the Fc-polypeptides having the desired domains operably linked to one another. The host is then used for the recombinant production of the multispecific polypeptide.

For example, a polypeptide fusion product can be produced from a vector in which one binding domain or a part thereof (e.g. a VH, VL, VHH or a VH/VL pair) is operably linked (e.g. directly, or via a CH1, Ck or C lambda constant region and/or hinge region) to the N-terminus of a CH2 domain, and the CH2 domain is operably linked at its C-terminus to the N-terminus a CH3 domain. Another binding domain or part thereof can be on a second polypeptide chain that forms a dimer, e.g. heterodimer, with the polypeptide comprising the first ABD.

CAR potentiator modules can then be produced in an appropriate host cell or by any suitable synthetic process. A host cell chosen for expression of the multispecific polypeptide is an important contributor to the final composition, including without limitation, the variation in composition of the oligosaccharide moieties decorating the protein in the immunoglobulin CH2 domain. The host cell may be of mammalian origin or may be selected from COS-1, COS-7, HEK293, BHK21, CHO, BSC-1, Hep G2, 653, QP2/0, 193, HeLa, myeloma, lymphoma, yeast, insect or plant cells, or any derivative, immortalized or transformed cell thereof. The host cell may be any suitable species or organism capable of producing N-linked glycosylated polypeptides, e.g., a mammalian host cell capable of producing human or rodent IgG type N-linked glycosylation.

In any of the embodiments described herein, a polynucleotide can include a polynucleotide that encodes a self-cleaving polypeptide, wherein the polynucleotide encoding the self-cleaving polypeptide is located between the polynucleotide encoding the CAR and a polynucleotide encoding a transduction marker (e.g., BFP). Exemplary self-cleaving polypeptides include 2A peptides from porcine teschovirus-1 (P2A), Thosea asigna virus (T2A), equine rhinitis A virus (E2A), foot-and-mouth disease virus (F2A), potyvirus 2A, cardiovirus 2A, or variant thereof.

Desired genes encoding CAR signalling or potentiator modules can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, mammalian artificial chromosomes (Vos, 1998, Curr. Op. Genet. Dev. 8:351-359), liposomes (Tarahovsky and Ivanitsky, 1998, Biochemistry (Mosc) 63:607-618), ribozymes (Branch and Klotman, 1998, Exp. Nephrol. 6:78-83), triplex DNA (Chan and Glazer, 1997, J. Mol. Med. 75:267-282), etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressed by the cell and, in certain instances, preferably heritable and expressed in its cell progeny.

### C) Cells Genetically Modified to Express a CAR

Provided herein are cells genetically modified to express a CAR (i.e. a CAR signaling module). The cells are preferably human cells. As used herein, the term “genetically modified” or “genetically engineered” refers to the addition of extragenetic material in the form of DNA or RNA into the total genetic material in a cell. The terms “genetically modified”, “engineered” and “modified”, when referring to cells, are used interchangeably. In some embodiments, a cell “genetically modified cells” and “modified cells” are used interchangeably. In some embodiments, a cell genetically modified to express a CAR is an immune effector cell. An immune effector cell may be characterized by exerting effector functions (e.g., cytotoxic cell killing activity, secretion of cytokines, induction of antibody-dependent cell cytotoxicity (ADCC) and/or complement dependent cytotoxicity (CDC).

Provided also are method of making a genetically engineered cell. The method can generally comprise genetically engineering a cell (e.g. a T cell, an NK cell) to express a nucleic acid or set of nucleic acids encoding a CAR signaling module of the disclosure. For example provided is a method for preparing a cell composition comprising introducing to a cell (e.g. a T cell, an NK cell) a nucleic acid or set of nucleic acids encoding a CAR signaling module of the disclosure. In one embodiment, provided is a method for preparing a cell composition, comprising: providing a cell, optionally an isolated a cell, optionally wherein the cell is a T cell or an NK cell; and introducing to the cell a nucleic acid or set of nucleic acids encoding a CAR signaling module of the disclosure, under conditions suitable such that the cell expresses at its surface the CAR signaling module. Optionally, a method further comprises a step of isolated the cell expressing the CAR signaling module. Optionally, a method further comprises a step of expanding (e.g. expanding the population of cells expressing the CAR signaling module) and/or growing in cell culture the cells expressing the CAR signaling module.

Immune cells of the disclosure can be autologous/autogeneic (“self”) or non-autologous (“non-self”, e.g., allogeneic, syngeneic or xenogeneic). “Allogeneic” refers to cells of the same species that differ genetically to a cell in comparison. “Syngeneic” refers to cells of a different subject that are genetically identical to the cell in comparison. “Xenogeneic” refers to cells of a different species to the cell in comparison. In some embodiments, modified cells of the disclosure are autologous or allogeneic.

In some embodiments, genetically modified cells include lymphocytes. In some embodiments, genetically modified cells include T cells, B cells, natural killer (NK) cells, monocytes/macrophages, and HSPC.

In a preferred embodiment, the cells that express a CAR signaling module are NK cells.

In other embodiments, the cells that express a CAR signaling module are T cells. T cells have a T-cell receptor (TCR) composed of two separate peptide chains (the a- (alpha-) and b- (beta-) TCR chains). Gamma-delta (gd) T cells represent a small subset of T cells that possess a distinct T cell receptor (TCR) made up of one g- (gamma-) chain and one d-(delta-) chain. CD3 is expressed on all mature T cells. T cells can further be classified into cytotoxic T cells (CD8+ T cells, also referred to as CTLs) and helper T cells (CD4+ T cells). Central memory T cells (TCM) refer to antigen experienced CTL that express CD62L or CCR7 and CD45RO and does not express or has decreased expression of CD45RA as compared to naive cells. Effector memory T cells (TEM) refer to an antigen experienced T-cell that does not express or has decreased expression of CD62L as compared to central memory cells and does not express or has decreased expression of CD45RA as compared to a naive cell. In some embodiments, effector memory T cells are negative for expression of CD62L and CCR7, compared to naive cells or central memory cells, and have variable expression of CD28 and CD45RA. Effector T cells are positive for granzyme B and perforin as compared to memory or naive T cells. Helper T cells assist other immune cells such as activating of cytotoxic T cells and macrophages and facilitating the maturation of B cells, among other functions. Helper T cells become activated when they are presented with peptide antigens by MHC class II molecules that are expressed on the surface of antigen presenting cells (APCs). Once activated, they divide rapidly and secrete cytokines that regulate or assist in the active immune response. Natural killer T (NKT) cells are a subset of T cells that co-express an ab T-cell receptor, but also express a variety of molecular markers that are typically associated with natural killer cells, such as CD16, and/or CD56.

Macrophages (and their precursors, monocytes) reside in every tissue of the body where they engulf apoptotic cells, pathogens and other non-self-components. Monocytes/macrophages express CD11b, CD68, CD11c, IL-4Ra, and/or CD163.

Immature dendritic cells (i.e., pre-activation) engulf antigens and other non-self-components in the periphery and subsequently, in activated form, migrate to T cell areas of lymphoid tissues where they provide antigen presentation to T cells. Dendritic cells express CD1 a, CD1b, CD1c, CD1d, CD21, CD35, CD39, CD40, CD86, CD101, CD148, CD209, and DEC-205.

Hematopoietic stem cells (HSC) refer to undifferentiated hematopoietic cells that are capable of self-renewal and differentiation into all other hematopoietic cell types. HSC are CD34+.

Hematopoietic progenitor cells (HPC) are derived from HSC and are capable of further differentiation into mature cell types. HPC can self-renew or can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T cells, B cells, and NK cells. HPC are CD24^{lo} Lin CD117+.

HSPC refer to a cell population having HSC and HPC. HSPC cell populations can be positive for CD34, CD43, CD45RO, CD45RA, CD59, CD90, CD109, CD117, CD133, CD166, HLA DR, or a combination thereof.

CAR T cells or CAR NK cells can off-the-shelf cells. The term “off-the-shelf” when referring to a cell as used herein means that the cells (i.e. allogeneic) are prepared in advance from healthy donors, validated and stored for until being administered as needed to a patient. Such off-the-shelf cells can be engineered to limit immune rejection, graft-versus-host disease (GvHD) and/or fratricide. Such engineering can include but is not limited to gene deletion (e.g. deletion of CD7 expression to prevent T cell fratricide, TCRs disruption to reduce GvHD induced by recognition of recipient antigens by donor TCRs, deletion of HLA class II expression to limit host immune rejection of allogeneic products (allo-rejection). Immune rejection occurs when a transplanted cell, tissue, or organ is not accepted by the body of a patient (recipient). Immune rejection is mediated by T cells and B cells of the adaptive immune system, along with NK cells of the innate immune system. For example, parts of the CAR can be recognized as foreign by T cells and NK cells of the host and targeted for destruction. Immune rejection of transplants can include hyperacute rejection, acute rejection, and chronic rejection. In some embodiments, hyperacute rejection occurs shortly after transplantation. In some embodiments, hyperacute rejection includes pre-existing antibodies reactive to the donor tissue. In some embodiments, hyperacute rejection includes severe systemic inflammatory responses following by blood clotting. In some embodiments, acute rejection occurs within one week after transplantation due to HLA antigen mismatch. In some embodiments, chronic rejection includes mismatched minor histocompatibility complex, resulting in long-term rejection of the transplant. In some embodiments, treatment for acute rejection includes re-transplantation or administration of chemotherapeutic immune suppressants (e.g. corticosteroids and calcineurin inhibitors). However, immune suppressants can lead to immunocompromise complications. In some embodiments, rejection of CAR modified cells by a patient includes no therapeutic response to the CAR therapy. In some embodiments, rejection of CAR modified cells by a patient includes destruction of the CAR modified cells by cytotoxic T cells and/or NK cells of the recipient.

### Methods of Collecting and Modifying Cells

In some embodiments, provided are methods for collecting, enriching for, culturing, and modifying cells to express a CAR of the disclosure. In some embodiments, T cells or NK cells are isolated from a sample such blood or blood-derived sample, an apheresis or a leukapheresis product. Exemplary samples include whole blood, peripheral blood mononuclear cells (PBMCs), leukocytes, bone marrow, thymus, cancer tissue, lymphoid tissue, spleen, or other appropriate sources. Sources of HSPC include umbilical cord blood, placental blood, and peripheral blood (see U.S. Pat. Nos. 5,004,681; 7,399,633; and 7,147,626; Craddock, et at, 1997, Blood 90(12):4779-4788. Well-known methods can be used for collection, anti-coagulation and processing etc. of blood samples.

In some embodiments, collected cells are washed, centrifuged, and/or incubated in the presence of one or more reagents, for example, to remove unwanted components, enrich for desired components, lyse or remove cells sensitive to particular reagents. The isolation an include one or more of various cell preparation and separation steps, including separation based on one or more properties, such as size, density, or resistance to particular reagents and/or affinity, e.g. immunoaffinity, to antibodies or other binding partners.

In some embodiments, one or more of the cell populations enriched, isolated and/or selected from a sample by the methods are cells that are positive for at least one marker (marker+) or express high levels of at least one marker (marker^{hi}) such as surface markers, or that are negative for at least one marker (marker-) or express relatively low levels of at least one marker (marker^{lo}). In some embodiments, the cell populations (such as T cells) are enriched for cells that are positive or expressing high surface levels of cell markers described elsewhere herein.

In some embodiments, T cells can be isolated from PBMCs by lysing the red blood cells and depleting the monocytes, for example, by centrifugation through a PERCOLL gradient. In some embodiments, a specific subpopulation of T cells, expressing CD3, CD28, CD4, CD8, CD45RA, and CD45RO is further isolated by positive or negative selection techniques. For example, enrichment of a T cell population by negative selection can be accomplished with a combination of antibodies directed to surface markers unique to the negatively selected cells. In some embodiments, cell sorting and/or selection occurs via negative magnetic immunoadherence or flow cytometry using a cocktail of monoclonal antibodies directed to cell surface markers present on the cells negatively selected. For example, to enrich for CD4+ cells by negative selection, a monoclonal antibody cocktail that typically includes antibodies to CD14, CD20, CD11b, CD16, HLA-DR, and CD8 can be used. Flow cytometry and cell sorting may also be used to isolate cell populations of interest for use in the present disclosure.

Following isolation and/or enrichment, cells can be expanded to increase the number of cells. In some embodiments, T cells can be activated and expanded before or after genetic modification to express a CAR, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US 2006/0121005.

Generally, the T cells are expanded by contact with a surface having attached thereto an agent that stimulates a CD3 TCR complex associated signal and a ligand that stimulates a co-stimulatory molecule on the surface of the T cells. In some embodiments, PBMCs or isolated T cells are contacted with a stimulatory agent and costimulatory agent, such as anti-CD3 and anti-CD28 antibodies, generally attached to a bead or other surface, in a culture medium with appropriate cytokines. In some embodiments, anti-CD3 and anti-CD28 antibodies attached to the same bead serve as a “surrogate” antigen presenting cell (APC). In some embodiments, the T cells may be activated and stimulated to proliferate with feeder cells and appropriate antibodies and cytokines using methods such as those described in U.S. Pat. Nos. 6,040,177; 5,827,642; and WO 2012/129514.

In some embodiments, NK cells can also be isolated from PBMCs by lysing the red blood cells and depleting the monocytes, for example, by centrifugation through a PERCOLL gradient. In some embodiments, a specific subpopulation of NK cells, expressing markers such as CD107, CD69, Sca-1 or Ly-6A/E, KLRG1 can be further isolated by positive or negative selection techniques.

In some embodiments, the isolating, incubating, expansion, and/or engineering steps are carried out in a sterile or contained environment and/or in an automated fashion, such as controlled by a computer attached to a device in which the steps are performed.

### CAR Detection and Control Elements

In some embodiments, a CAR signaling module or a cell that expresses such can include one or more tags to activate, promote proliferation of, enrich for, isolate, track, deplete and/or eliminate genetically modified cells in vitro, in vivo, and/or ex vivo. A tag may be for example a unique synthetic peptide sequence fused to a CAR, or that is part of a CAR, to which a cognate binding molecule (e.g., ligand, antibody, or other binding partner) is capable of specifically binding, where the binding property can be used to activate, promote proliferation of, detect, enrich for, isolate, track deplete and/or eliminate the tagged CAR.

Tags that can be included in a CAR may include, for example, His tag (Life Technologies) (SEQ ID NO: 192), Flag tag (Pierce Antibodies) (SEQ ID NOs: 193-195), Xpress tag (Pierce Antibodies) (SEQ ID NO: 196), Avi tag (Pierce Antibodies) (SEQ ID NO: 197), Calmodulin binding peptide tag (Santa Cruz Biotechnology) (SEQ ID NO: 198), Polyglutamate tag (SEQ ID NO: 199), HA tag (Pierce Antibodies) (SEQ ID NOs: 200-202), Myc tag (Santa Cruz Biotechnology) (SEQ ID NO: 203), Strep tag (which refers to the original STREP tag (SEQ ID NO: 204), STREP tag II (SEQ ID NO: 205) (IBA); see, e.g., U.S. Pat. No. 7,981,632), Softag 1 (SEQ ID NO: 206), Softag 3 (SEQ ID NO: 207), and V5 tag (SEQ ID NO: 208). Conjugate binding molecules that specifically bind tag sequences herein are commercially available.

In some embodiments, a cell made to express a CAR signaling module (e.g. a CAR T cell or a CAR NK cell) or CAR molecule can include elements to regulate the activity of the CAR to manage toxicity or tune the magnitude of CAR activity. In some embodiments, molecular safety switches in the form of suicide genes, such as inducible-caspase-9 (iCASP9), herpes simplex virus thymidine kinase (HSV-TK), or truncated surface receptors (e.g., tEGFR) can be used. A suicide gene encodes a molecule which allows selective destruction of cells expressing this molecule upon administration of a nontoxic prodrug or antibody. For example, the iCASP9 system is based on the fusion of caspase 9 and a drug-sensitive FK-modified binding protein. Upon being exposed to the synthetic molecule AP1903, the fusion protein dimerizes and leads to the rapid apoptosis of cells expressing the fusion protein. In some embodiments, the use of an inhibitory CAR (iCAR) can selectively limit cytokine secretion, cytotoxicity, and/or proliferation induced through an activating CAR and can be utilized to protect healthy tissue from anti-NK CAR-mediated destruction.

In some embodiments, CAR expressing cells administered to a subject can be controlled by depleting (eliminating) the CAR expressing cells at a desired time after administration. A CAR including at least one tag and/or transduction marker can be depleted using a respective cognate binding molecule that binds the tag or transduction marker. In some embodiments, the present disclosure provides a method for depleting a CAR modified cell by using a cognate binding molecule specific for a tag or transduction marker (e.g., an antibody) or by using a second modified cell expressing a CAR that has specificity for the tag or transduction marker. In some embodiments, a cognate binding molecule includes a depletion agent specific for a tag or transduction marker. For example, if tEFGR is used as the transduction marker, then an anti-tEFGR binding domain (e.g., antibody, scFv) fused to or conjugated to a cell-toxic reagent (such as a toxin or radiometal) may be used, or an anti-tEFGR/anti-CD3 bispecific scFv, or an anti-tEFGR CAR T cell may be used.

Some embodiments provide for elements that can be used to turn on, induce, or increase the activity of a CAR. Systems have been developed that allow pharmacologic induction of CAR expression. In some embodiments, a system can include a bipartite receptor system containing separate antigen-targeting and signal transduction polypeptides, each containing an extracellular dimerization domain. T cell activation is antigen dependent but can only be achieved in the presence of a dimerizing drug, rapamycin. Regulation of CAR T cell activity is reviewed in Brandt et al. (2020) Frontiers in Immunology, 11: 326.

### Activity Assays

Any relevant assay or test well known to those in the art may be used to determine whether a cell expressing a CAR signaling module can bind a CAR potentiator module, or can bind and/or kill a target cell a tumor antigen. In some embodiments, a CAR modified cell can bind and/or kill a target cell when the CAR modified cell is activated. Assessment of CAR modified cell activation includes: (i) induction of CD137 (4-1BB) expression on CAR modified cells upon binding the tumor antigen marker on the surface of the tumor cell optionally when CAR modified cells further bind NKp46 on the surface of NK cell; (ii) secretion of cytokines including IL-2, IFN gamma, tumor necrosis factors (e.g. TNF alpha), IL-15, and IL-13, and/or (iii) cytotoxicity towards tumor cells expressing the tumor antigen marker.

For example, assessing CAR modified cell function can include the following. Target cells genetically modified to express a tumor surface marker (e.g. EGFR, CD20, HER2, etc.) can be contacted with a CAR modified cells (effector cells expressing a CAR signaling module) and CAR potentiator module having a binding domain specific to said tumor surface marker, In a ratio of CAR modified cells:target cells of 2:1. In some embodiments, a ratio of CAR modified cells: target cells of 1:1, 2:1, 3:1, 4:1, 5:1, 6:1, 7:1, 8:1, 9:1, 10:1. In some embodiments, target cells include RAJI cells, Daudi cells, or generally any suitable available tumor cells line.

In some embodiments, induction of CD137 expression on cells made to express a CAR signaling module can be measured by flow cytometry using antibodies that recognize CD137, when the cells are brought into contact with a target cell in the presence of the CAR potentiator. The percentage of CD137+ CAR modified cells can be measured by flow cytometry after the modified cells have been contacted with a labeled antibody that binds CD137. Antibodies that bind CD127 are commercially available, including a rabbit monoclonal anti-human CD137 antibody (BLR051F; Abeam, Cambridge, UK), a mouse monoclonal 4B4-1 anti-human CD137 antibody (Thermo Fisher, Waltham, MA), and mouse monoclonal BBK-2 anti-human CD137 antibody (Santa Cruz Biotechnology, Dallas, TX).

In some embodiments, secretion of cytokines from cells can be measured by assays known in the art, when the cells are brought into contact with a target cell in the presence of the CAR potentiator. For example, secretion of cytokines can be measured by ELISA, Western blot, flow cytometry, single cell multiplex cytokine profiling, and high throughput single cell 3′ mRNA transcriptome sequencing. In some embodiments the assays use antibodies that specifically bind to particular cytokines. In some embodiments, the assays use flow cytometry to measure intracellular staining of cytokines. In some embodiments, the assays use cellular barcoding of 3′ mRNA and high-throughput sequencing. In some embodiments, the cytokines measured include II-2, IFN gamma, and tumor necrosis factor (e.g., TNF alpha). Cytokine assays are further described in Wilkie et al. (2008) J Immunol. 180:4901-4909, Jena et al. (2014) Curr Hematol Malig Rep. 9:50-56, Kaiser et al. (2015) Cancer Gene Ther. 22:72-78, Xue et al. (2017) J Immunother Cancer 5:85, and Xhangolli et al. (2019) Genomics Proteomics Bioinformatics. 17(2): 129-139.

In some embodiments, the cytotoxic activity of cells can be measured by flow cytometry, when the cells are brought into contact with a target cell in the presence of the CAR potentiator. In some embodiments, a labeled antibody against a surface marker of the targeted tumor cells can be used to quantify the amount of target cells in the presence or absence of modified cells and CAR potentiator module by flow cytometry. In some embodiments, cytotoxicity assays include chromium-51 release assay or similar assay using a non-radioactive reporter such as enhanced green fluorescent protein (GFP)-firefly luciferase fusion (Xiong et al. (2018) J Immunol 200(1): Supplement 1, 179.2), lactate dehydrogenase (LDH) release assay, real time cell analyzing systems that use gold microelectrode biosensors to quantify cell viability by electrical impedance (e.g., Sener et al. (2017) Exp Ther Med. 14(3): 1866-1870), and real time systems based on probes such as propidium iodide and SYTOX Green based on compromised cellular membrane integrity. In a release assay, tumor cell killing can be quantified by the amount of chromium-51 (preloaded into target cells before contact with a modified cell) or LDH released from lysed cells. Target cell cytotoxicity can be calculated with the following formula: % of cytotoxicity=100° [(CAR-T: target cells−CAR-T cells alone−target cells alone)/(maximum target cell lysis−target cells alone without lysis buffer)].

The CAR potentiator module can also be tested for its biological activity, e.g. antigen ability to elicit target cell lysis and/or specific signaling activities, in the presence or in the absence of cells made to express a CAR signaling module. It will be appreciated that when the specific contribution or activity of one of the components of the CAR potentiator module is to be assessed (e.g. an NKp46 binding domain, an antigen-of-interest binding domain, an Fc domain, cytokine receptor binding domain, etc.), the CAR potentiator module can be produced in a suitable format which allows for assessment of the component (e.g. binding domain) of interest. The present disclosure also provides such methods, for use in testing, assessing, making and/or producing a CAR potentiator module. For example, where the contribution or activity of a cytokine is assessed, the CAR potentiator module can be produced as a protein having the cytokine and another protein in which the cytokine is modified or absent (e.g. wherein the two CAR potentiator modules otherwise have the same or comparable structure), and tested in an assay of interest. In another example, where the contribution or activity of an anti-NKp46 binding domain is assessed, the CAR potentiator module can be produced as a protein having the NKp46 binding domain and another CAR potentiator module in which said binding domain is absent or is replaced by a binding domain that does not bind NKp46 (e.g. a binding domain that binds an antigen not present in the assay system), wherein the two CAR modules otherwise have the same or comparable structure, and compared in an assay of interest. In another example, where the contribution or activity of an antigen of interest (e.g. tumor antigen) binding domain is assessed, the CAR potentiator module can be produced as a protein having the binding domain an another protein in which said binding domain is absent or is replaced by a binding domain that does not bind the tumor antigen of interest (e.g. a binding domain that binds an antigen not present in the assay system, a binding domain that bind to a different tumor antigen), wherein the two CAR potentiator module otherwise have the same or comparable structure, and the two CAR potentiator module are tested in an assay of interest.

In some aspect, the CAR potentiator module is capable of binding a CAR signaling module expressed by a modified cell (e.g. a modified NK cell, a modified T cell, etc.) and further induces activation of said modified cell, when incubated in the presence of a tumor cell that express a tumor antigen recognized by the CAR potentiator module. In another aspect, the CAR potentiator module is capable of inducing activation of an NKp46-expressing cell (e.g. a NK cell) when incubated in the presence of the NKp46-expressing cell (e.g. a NK cell) and a tumor cell that express a tumor antigen recognized by the CAR potentiator module. Such activation of an NKp46-expressing cell can occurs without any involvement of a cell expressing a CAR signaling module.

In some embodiments, NK cell activation or signaling is characterized by an increased expression of a cell surface marker of activation (e.g. CD107a, CD107b, CD69, KLRG1, etc.).

In some embodiments, the CAR potentiator module is capable of inducing an increase of CD137 present on the cell surface of an NKp46 and/or CD16-expressing cell (e.g. a NK cell) when said CAR potentiator module is incubated in the presence of the NKp46 and/or CD16-expressing cell (e.g. a NK cell), in the absence of modified cells expressing CAR signaling molecule, and optionally in the presence of tumor cells.

In some embodiments, a CAR potentiator module that comprise a cytokine receptor binding domain is capable of activating or enhancing the proliferation of NK cells by at least 10-fold, at least 50 fold or 100 fold compared to the same CAR potentiator module lacking the cytokine receptor binding domain (e.g. a cytokine, a CD122 binding domain). Optionally, the CAR potentiator module displays an EC50 for activation or enhancing the proliferation of NK cells that is at least 10-fold, 50-fold or 100-fold lower than its EC50 for activation or enhancing the proliferation of CD25-expressing T cells.

In some embodiments, the CAR potentiator module is capable of activating or enhancing the proliferation of NK cells over CD25-expressing T cells, by at least 10-fold, at least 50-fold, or at least 100-fold. Optionally, the CD25-expressing T cells are CD4 T cells, optionally Tregs, or CD8 T cells.

Activation or enhancement of proliferation via cytokine receptor in cells (e.g. NK cells, CD4 T cells, CD8 Tcells or Treg cells) by the CAR potentiator module comprising a cytokine receptor binding domain can be determined by measuring the expression of pSTAT or the cell proliferation markers (e.g. Ki67) in said cells following the treatment with the multispecific protein. Activation or enhancement of proliferation via the IL-2R pathway in cells (e.g. NK cells, CD4 T cells, CD8 Tcells or Treg cells) by the CAR potentiator module comprising a CD122 binding domain can be determined by measuring the expression of pSTAT5 or the cell proliferation marker Ki67 in said cells following the treatment with the multispecific protein. IL-2 and IL-15 lead to the phosphorylation of the STAT5 protein, which is involved in cell proliferation, survival, differentiation and apoptosis. Phosphorylated STAT5 (pSTAT5) translocates into the nucleus to regulate transcription of the target genes including the CD25. STAT5 is also required for NK cell survival and NK cells are tightly regulated by the JAK-STAT signaling pathway. In one aspect, the CAR potentiator is capable of inducing STAT5 signaling in an NKp46-expressing cell (e.g. an NK cell) when the protein is incubated in the presence of an NKp46-expressing cell (e.g. purified NK cells). In one aspect, the CAR potentiator is capable of causing an increase of expression of pSTAT5 in NK cells over CD25-expressing T cells, by at least 10-fold, at least 50-fold, or at least 100-fold. Optionally the CAR potentiator displays an EC50 for induction of expression of pSTAT5 in NK cells that is at least 10-fold, 50-fold or 100-fold lower than its EC50 for induction of expression of pSTAT5 in CD25-expressing T cells. Similarly, cytokine receptor signal transduction can also be assessed for other cytokine/cytokine receptor pairs, such as IL-15 (STAT5), IL-21 (STAT3), IL-27 (STAT1), IL-12 (STAT4), etc.

The intrinsic activity of CAR potentiator modules (i.e. without being bound to CAR signaling modules) can be measured for instance by bringing NKp46-expressing cells (or CD25-expressing cells, depending on the assay) into contact with the CAR potentiator modules, optionally further in presence of tumor cells. In some embodiments, activity is measured for example by bringing target cells and NK cells (i.e. NKp46-expressing cells) into contact with one another, in presence of the CAR potentiator module. The NKp46-expressing cells within a population of peripheral blood mononuclear cells (PBMC). In one example, the CAR potentiator module can be assessed for the ability to cause a measurable increase in any property or activity known in the art as associated with NK cell activity, respectively, such as a marker of cytotoxicity (CD107a) or cytokine production (e.g. IFN-gamma or TNF-alpha), increases in intracellular free calcium levels, the ability to lyse target cells, for example in a redirected killing assay, etc.

In the presence of tumor cells and NK cells that express NKp46, the CAR potentiator module will be capable of causing an increase in a property or activity associated with NK cell activity (e.g. activation of NK cell cytotoxicity, CD107a expression, IFN gamma production, killing of target cells) in vitro. For example, a CAR potentiator module can be selected based on its ability to increase NK cell activity by more than about 20%, preferably by least about 30%, at least about 40%, at least about 50%, or more compared to that achieved with the same effector: tumor cell ratio with the same NK cells and target cells that are not brought into contact with the CAR potentiator module, as measured by an assay that detects NK cell activity, e.g., an assay which detects the expression of an NK cell activation marker or which detects NK cell cytotoxicity, e.g., an assay that detects CD107 or CD69 expression, IFN gamma production, or a classical in vitro chromium release test of cytotoxicity. Examples of protocols for detecting NK cell activation and cytotoxicity assays are described in the Examples herein, as well as for example, in Pessino et al, J. Exp. Med, 1998, 188 (5): 953-960; Sivori et al, Eur J Immunol, 1999. 29:1656-1666; Brando et al, (2005) J. Leukoc. Biol. 78:359-371; EI-Sherbiny et al, (2007) Cancer Research 67(18):8444-9; and Nolte-'t Hoen et al, (2007) Blood 109:670-673). In a classical in vitro chromium release test of cytotoxicity, the target cells are labeled with ⁵¹Cr prior to addition of NK cells, and then the killing is estimated as proportional to the release of ⁵¹Cr from the cells to the medium, as a result of killing. Optionally, a CAR potentiator module can be selected for or characterized by its ability to have greater ability to induce NK cell activity towards tumor cells, i.e., lysis of tumor cells compared to a conventional human IgG1 antibody that binds to the same tumor antigen, as measured by an assay of NK cell activity (e.g. an assay that detects NK cell-mediated lysis of target cells that express the tumor antigen).

In some embodiments, a CAR potentiator module alone or in combination with a CAR signaling module expressed by a cell (e.g. a modified T cell, a modified NK cell) can for example be characterized by:
(a) Capable of inducing cytokine receptor (e.g. CD122) signaling (e.g. as determined by assessing STAT signaling, for example assessing STAT phosphorylation) in an NKp46-expressing cell (e.g. a NK cell) when the CAR potentiator module is incubated in the presence of an NKp46-expressing cell (e.g. purified NK cells), and when said CAR potentiator module comprises a cytokine receptor binding domain/cytokine moiety;
(b) Being capable of inducing NK cells that express NKp46 (and optionally further CD16) to lyse tumor cells, when incubated in the presence of NK cells and tumor cells; and
(c) Lack of NK cell activation or cytotoxicity and/or lack of agonist activity at NKp46 when incubated with NK cells (optionally CD16-negative NK cells, NKp46-expressing cells that do not express CD16a), in the absence of tumor cells, optionally wherein the NK cells are purified NK cells, when the CAR potentiator module lack the cytokine receptor binding domain (e.g. CD122 binding domain) or comprise an inactivated cytokine receptor binding domain.

### D) Compositions and Formulations

The CAR potentiator module proteins and the cells genetically modified ex vivo to express a CAR signaling module can each be formulated for administration to subjects, either together or in separate formulations.

A pharmaceutical composition refers to a composition formulated in pharmaceutically-acceptable or physiologically acceptable solutions for administration to a subject, either alone, or in combination with one or more other modalities of therapy. It will also be understood that, if desired, the compositions may be administered in combination with one or more other additional modalities of therapy. It will also be understood that, if desired, the compositions may be administered in combination with other agents as well, such as, e.g., cytokines growth factors, hormones, small molecules, chemotherapeutics, pro-drugs, drugs, antibodies, or other various pharmaceutically active agents, generally provided that the additional agents do not adversely affect the cells or the ability of the composition to deliver the intended therapy. It will also be understood that, if desired, the CAR potentiator module proteins and the cells genetically modified ex vivo to express a CAR signaling module can be administered in absence of combined administration of any of the aforementioned additional modalities of therapy. In some embodiment, the high anti-tumor efficacy of CAR potentiator module proteins and the cells genetically modified ex vivo to express a CAR signaling module permit them to be administered as a monotherapy system (without combined administration of further anti-cancer agents).

The phrase pharmaceutically acceptable refer to those compounds, materials, compositions, and/or dosage forms which are, within the scope of sound medical judgment, suitable for use in contact with the tissues of patients without excessive toxicity, irritation, allergic response, or other problem or complication, commensurate with a reasonable benefit/risk ratio.

Exemplary carriers include saline, buffered saline, physiological saline, water, Hanks' solution, Ringer's solution, Nonnosol-R (Abbott Labs), PLASMA-LYTE A (Baxter Laboratories, Inc., Morton Grove, IL), glycerol, ethanol, and combinations thereof. In some embodiments, carriers can be supplemented with human serum albumin (HSA) or other human serum components or fetal bovine serum. In some embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols, amino acids, organic sugars or sugar alcohols, PEG, sulfur-containing reducing agents, bovine serum albumin, gelatin or immunoglobulins, polyvinylpyrrolidone, and saccharides.

Where necessary or beneficial, compositions or formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens, catechol, resorcinol, cyclohexanol, and 3-pentanol.

Therapeutically effective amounts of modified cells (i.e. cells expressing CAR signaling module according to the disclosure) within compositions or formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In compositions and formulations disclosed herein, cells are generally in a volume of a liter or less, 500 ml or less, 250 ml or less, or 100 ml or less. Hence the density of administered cells is typically greater than 10⁴ cells/ml, 10⁷ cells/ml, or 10⁸ cells/ml.

In some embodiments, compositions and formulations can include one or more genetically modified cell type (e.g., modified T cells, NK cells). The different populations of genetically modified cells can be provided in different ratios.

The cell-based compositions and formulations can be prepared for administration by, e.g., injection, infusion, perfusion, or lavage. Cell compositions are typically administered intravenously and can thus be advantageously formulated for intravenous delivery. Alternatively compositions can be formulated for bone marrow, intradermal, intraarterial, intranodal, intralymphatic, intraperitoneal, intralesional, intraprostatic, intrathecal, intratumoral, intramuscular, intravesicular, and/or subcutaneous injection.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

In some instances, it can be useful to cryopreserve cells or cell formulations of the disclosure. As used herein, “cryopreserving,” refers to the preservation of cells by cooling to sub zero temperatures, such as (typically) 77 K or −196° C. (the boiling point of liquid nitrogen). Cryoprotective agents are often used at sub-zero temperatures to ameliorate or prevent cell damage due to freezing at low temperatures or warming to room temperature. Cryoprotective agents and optimal cooling rates can protect against cell injury. Cryoprotective agents which can be used include dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. N.Y. Acad. Sci., 1960; 85: 576), and polyethylene glycol (Sloviter and Ravdin, Nature, 1962; 196: 48). In some embodiments, the cooling rate is 1° to 3° C./minute. After at least two hours, the cells reach a temperature of −80° C. and can be placed directly into liquid nitrogen (−196° C.) for permanent storage such as in a long-term cryogenic storage vessel.

In some embodiments, provided are pharmaceutical compositions comprising CAR potentiator module according to the disclosure and pharmaceutically acceptable carrier. The final form of such a pharmaceutical composition depends on the intended mode of administration and therapeutic or diagnostic application. The pharmaceutical carrier can be any compatible, nontoxic substance suitable to deliver the compounds to the patient. Pharmaceutically acceptable carriers are well known in the art and include, for example, aqueous solutions such as (sterile) water or physiologically buffered saline or other solvents or vehicles such as glycols, glycerol, oils such as olive oil or injectable organic esters, alcohol, fats, waxes, and inert solids. A pharmaceutically acceptable carrier may further contain physiologically acceptable compounds that act for example to stabilize or to increase the absorption of the compounds Such physiologically acceptable compounds include, for example, carbohydrates, such as glucose, sucrose or dextrans, antioxidants, such as ascorbic acid or glutathione, chelating agents, low molecular weight proteins or other stabilizers or excipients One skilled in the art would know that the choice of a pharmaceutically acceptable carrier, including a physiologically acceptable compound, depends, for example, on the route of administration of the composition pharmaceutically acceptable adjuvants, buffering agents, dispersing agents, and the like, may also be incorporated into the pharmaceutical compositions.

Pharmaceutical composition comprising CAR potentiator modules of the disclosure can be prepared to be administered by the same route as the pharmaceutical composition comprising modified cells as defined above. In some embodiments, a pharmaceutical composition comprising CAR potentiator modules of the disclosure can be formulated for bone marrow, intravenous, intradermal, intraarterial, intranodal, intralymphatic, intraperitoneal, intralesional, intraprostatic, topical, intrathecal, intratumoral, intramuscular, intravesicular, and/or subcutaneous injection. For example a composition for intravenous infusion may comprise 100 to 500 ml of sterile 0.9% NaCl or 5% glucose, optionally supplemented with a 20% albumin solution and 1 mg to 10 mg of CAR potentiator modules of the disclosure, depending on the particular type of CAR potentiator modules and its required dosing regimen.

### Methods of Treatment

In one embodiment, provided is the use of the modified cells expressing a CAR signaling domain of the disclosure as a medicament or as an active component or active substance in a medicament. Also provided are methods of treating disease in an individual comprising administering to the individual the modified cells expressing a CAR signaling domain of to the disclosure. In some embodiments, provided is the combined use of (a) modified cells expressing CAR signaling domain described herein and (b) CAR potentiator module proteins described herein, for the treatment, prevention or diagnosis of a disease in a mammal in need thereof (e.g. cancer). As used herein, combined administration (co-administration) includes simultaneous administration of the compositions in the same or different dosage form, or separate administration of the compositions (e.g., sequential administration). Thus, a composition of modified cells can be used in combination with a potentiator module (e.g., as a pharmaceutical composition comprising purified or isolated potentiator module protein). For example, modified cells and an potentiator module protein can be simultaneously administered in a single formulation. Alternatively, the modified cells and the potentiator module protein can be formulated for separate administration and are administered concurrently or sequentially. In one embodiment, the modified cells are administered before the potentiator module. In one embodiment the potentiator module is administered before the modified cells.

Examples of the disease or cancer to be treated can include: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma. Other exemplary disorders that can be treated include hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sezary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukaemia (T-Lbly/T-ALL).

Provided also are methods for preparing a first pharmaceutical composition containing modified cells expressing a CAR signaling module as described herein. Provided also are methods for preparing a second pharmaceutical composition containing CAR potentiator module as described herein. In some embodiments the pharmaceutical compositions as described herein are suitable for administration to a patient (e.g., by intravenous of subcutaneous injection). Such methods or processes at least comprise the step of mixing the compound with a pharmaceutically acceptable carrier.

In some embodiments, provided is the use of cells expressing a CAR signaling domain, for use in the treatment, prevention of a disease in a mammal in need thereof, wherein the treatment is in combination with a CAR potentiator module. In some embodiments, provided are cells expressing CAR signaling domain, for use in the treatment, prevention of a disease in a mammal in need thereof, wherein the treatment is in combination with a CAR potentiator module. In one embodiment the disease is cancer.

In some embodiments herein, modified cells expressing CAR signaling modules according to the disclosure can be administered at a dose range from 10⁶-10¹² cells/kg body weight. In some embodiments, useful doses can include 10⁶×cells/kg, 10⁷ cells/kg, 10⁸ cells/kg, 10⁹ cells/kg, 10¹⁰ cells/kg, 10¹¹ cells/kg, 10¹² cell/kg, or more. The modified cells (e.g. modified T cells, modified NK cells, etc.) can be allogeneic, syngeneic, xenogeneic, or autologous to the patient undergoing therapy. Example of useful doses to administer can range from 0.1 to 5 μg/kg or from 0.5 to 1 μg/kg. In other examples, a dose can include 1 μg/kg, 15 μg/kg, 30 μg/kg, 50 μg/kg, 70 μg/kg, 90 μg/kg, 100 μg/kg, 150 μg/kg, 250 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

In some embodiments herein, CAR potentiator modules according to the disclosure can be administered at a dose range from 1 μg/kg to 10 mg/kg body weight. In some embodiments, useful dose can include a range from 1 μg/kg to 1 mg/kg, 0.1 mg/kg to 1 mg/kg or 0.05 mg/kg to 0.5 mg/kg.

Therapeutically effective amounts can be achieved by administering single or multiple doses of modified cells expressing CAR signaling modules and CAR potentiator modules during the course of a treatment regimen, e.g. every week, every month, etc. In one embodiment, a single dose of CAR signaling module and a single dose of modified cells is administered to an individual.

The compositions and formulations as described herein can be administered by injection, transfusion, implantation or transplantation. In some embodiments, the compositions and formulations are administered parenterally. “Parenteral administration” and “administered parenterally” refer to modes of administration other than enteral and topical administration, usually by injection, and includes intravascular, intravenous, intramuscular, intraarterial, intrathecal, intracapsular, intratumoral, intraperitoneal, and subcutaneous injection and infusion. In some embodiments, the composition and formulations may be administered in combination with (e.g. before, simultaneously or following) any number of relevant treatment modalities, such as chemotherapeutic agents, radiation, immunosuppressive or immunoablative agents or anti-inflammatory agents.

In some embodiments, the modified cells expressing CAR signaling modules as described herein are administered to a patient in need thereof. In some embodiments, the modified cells expressing CAR signaling modules are able to replicate in vivo, resulting in a long-term persistence that can lead to sustained therapy. In some embodiments, the modified cells expressing CAR signaling modules can undergo robust cell expansion and can persist for an extended amount of time. In other embodiments, the modified cells expressing CAR signaling modules can include a suicide gene construct able to induce the depletion of the modified cells when the treatment scheme is completed.

In some embodiments, provided are methods to treat, prevent or more generally affect a predefined condition in an individual or to detect a certain condition using or administering modified cells expressing CAR signaling modules as described herein and CAR potentiator module as described herein, or pharmaceutical compositions comprising the same.

In some embodiments, provided is a method for stimulating a T cell or NK cell-mediated immune response to a tumor cell population or tissue in a patient. In some embodiments, such a method comprises administering to a patient an effective amount of modified cells expressing CAR signaling module and administering to a patient an effective amount of CAR potentiator modules, wherein said CAR potentiator modules comprise a binding domain that specifically binds to the tumor cell population.

### Kits

Any of the composition described herein may be included in a kit. In some embodiments, one or more of the following an be provided in a kit: cells to be genetically modified to express a CAR signaling module, including immune cells (e.g. T cells, NK cells), reagents suitable for expanding the cells to be modified, including media, aAPCs, growth factor, and antibodies; reagents suitable for introducing nucleic acids encoding a CAR signaling module into cells, including reagents for transfection and/or transduction of cells, modified cells expressing CAR signaling module; reagents to cryopreserve cells; CAR signaling module expression constructs; reagents to generate CAR signaling module expression construct including enzymes, polymerases, and primers; reagents suitable for characterizing the modified cells expressing CAR signaling module, including antibodies to sort or detect the CAR signaling module; CAR signaling module.

The kits may include one or more suitably aliquoted reagents to generate compositions of the disclosure. The components of the kits may be packaged either in aqueous media or in lyophilized form. The container means of the kits may be composed of at least one vial, test tube, flask, bottle, syringe, or other container means, into which a component may be placed. Where there is more than one component in the kit, the kit also generally contain one or more additional container into which the additional components may be separately placed. However, various combinations of components may be included in a container. A kit typically include a means for containing a CAR potentiator module, and another means for containing cells expressing CAR signaling module.

In some embodiments, provided are kits comprising (i) cells modified to express CAR a signaling module and (ii) CAR potentiator module proteins. In one aspect, provided herein are kits which include: (i) a pharmaceutical composition containing cells modified to express CAR a signaling module and (ii) a pharmaceutical composition containing a CAR potentiator module proteins. Each of (i) and (ii) can be specified as being in a container, for example in a vial, or optionally for modified cells, a bag or other non-rigid cell culture container.

### EXAMPLES

### Example 1: Construction of CAR NK Cells

### Preparation of CAR Potentiator Modules:

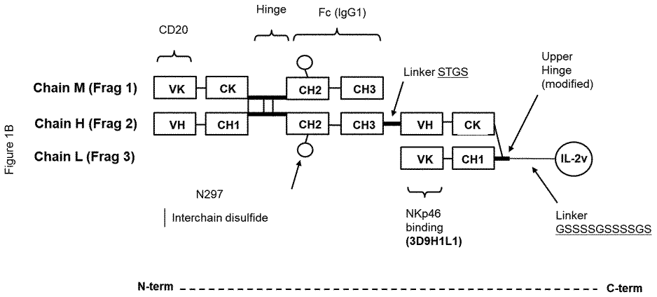

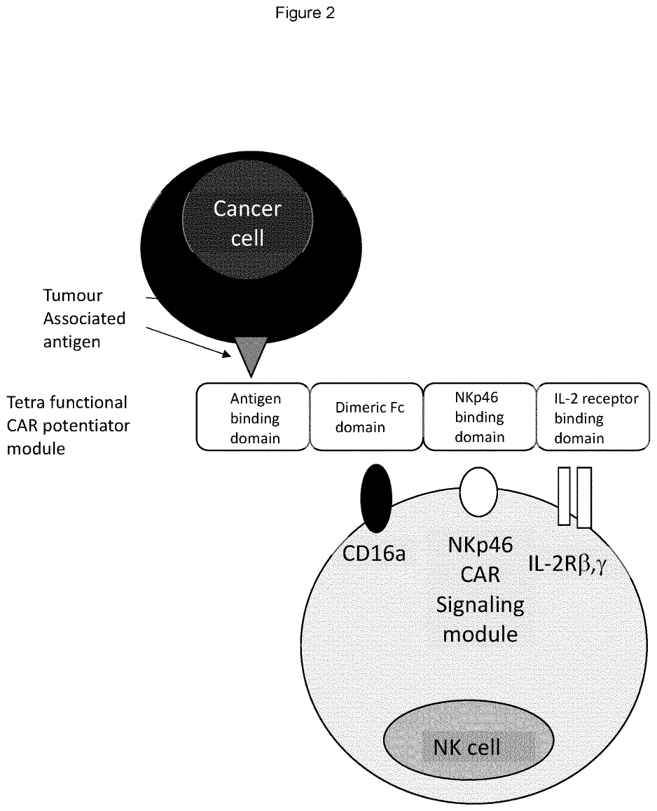

The domain structure of an exemplary “F25” format CAR potentiator module protein used in the examples is shown in FIG. 3A. The domain structure of an exemplary “T5” format CAR potentiator module protein further comprising a cytokine is shown in FIGS. 2 and 3C. FIG. 2 shows domain linkers such as hinge and glycine-serine linkers, and interchain disulfide bridges for the exemplary format T5. The domain structure of the exemplary “T6” format, having a N297S mutation to substantially abolish CD16a binding but otherwise equivalent to format T5, is shown in FIG. 3D. To build the T5 format chain L (also referenced as chain 3) the CK domain normally associated with the NKp46-1 VK domain in the NKp46 ABD was replaced by CH1 domain. In order to ensure a correct pairing between Chain L (chain 3) and Chain H (chain 1) and formation of a proper disulfide bond between H and L chains, the upper-hinge residues of human IgG1 were added at the C-terminus of CH1 domain of chain L upstream of the linker connecting chain L to IL-2v. Other protein formats are shown within FIGS. 3B and 3E.

The sequences encoding each polypeptide chain for each multispecific antigen-binding protein were inserted into the pTT-5 vector between the HindIII and BamHI restriction sites. The three vectors (prepared as endotoxin-free midipreps or maxipreps) were used to cotransfect EXPI-293F cells (Life Technologies) in the presence of PEI (37° C., 5% CO₂, 150 rpm). The cells were used to seed culture flasks at a density of 1×106 cells per ml (EXP1293 medium, Gibco). As a reference, for the “T5” constructs, we used a DNA ratio of 0.1 μg/ml (polypeptide chain I), 0.4 μg/ml (polypeptide chain II), or 0.8 μg/ml (polypeptide chain III). Valproic Acid (final concentration 0.5 mM), glucose (4 g/L) and tryptone N1 (0.5%) were added. The supernatant was harvested after six days after and passed through a Stericup filter with 0.22 μm pores.

The CAR potentiator modules were purified from the supernatant following harvesting using rProtein A Sepharose Fast Flow (GE healthcare, reference 17-1279-03.) Size Exclusion Chromatography (SEC) purifications were then performed and the proteins eluted at the expected size were finally filtered on a 0.22 μm device.

The amino acid sequence of the polypeptide chains of the multispecific proteins produced are shown below in Table D.

TABLE D

| Sample | Frag M (chain 2) | Frag H (chain 1) | Frag L (chain 3) |
|---|---|---|---|
| CD20- | MSVPTQVLGLLLLWLTDA | MEWSWVFLFFLSVTTGVHSEVQLVE | MSVPTQVLGLLLL |
| T5- | RCEIVLTQSPATLSLSPG | SGGGLVQPDRSLRLSCAASGFTFHD | WLTDARCDIQMTQ |
| NKp46-1- | ERATLSCRASQSVSSYL | YAMHWVRQAPGKGLEWVSTISWNS | SPSSLSASVGDRV |
| IL2v2A | AWYQQKPGQAPRLLIYD | GTIGYADSVKGRFTISRDNAKNSLYL | TITCRASQDISNYL |
| | ASNRATGIPARFSGSGS | QMNSLRAEDTALYYCAKDIQYGNYY | NWYQQKPGKAPKL |
| | GTDFTLTISSLEPEDFAV | YGMDVWGQGTTVTVSSASTKGPSV | LIYYTSRLHSGVPS |
| | YYCQQRSNWPITFGQGT | FPLAPSSKSTSGGTAALGCLVKDYFP | RFSGSGSGTDFTF |
| | RLEIKRTVAAPSVFIFPPS | EPVTVSWNSGALTSGVHTFPAVLQS | TISSLQPEDIATYFC |
| | DEQLKSGTASVVCLLNN | SGLYSLSSVVTVPSSSLGTQTYICNV | QQGNTRPWTFGG |
| | FYPREAKVQWKVDNALQ | NHKPSNTKVDKRVEPKSCDKTHTCP | GTKVEIKASTKGPS |
| | SGNSQESVTEQDSKDST | PCPAPELLGGPSVFLFPPKPKDTLMI | VFPLAPSSKSTSG |
| | YSLSSTLTLSKADYEKHK | SRTPEVTCVVVDVSHEDPEVKFNWY | GTAALGCLVKDYF |
| | VYACEVTHQGLSSPVTK | VDGVEVHNAKTKPREEQYNSTYRVV | PEPVTVSWNSGAL |
| | SFNRGECDKTHTCPPCP | SVLTVLHQDWLNGKEYKCKVSNKAL | TSGVHTFPAVLQS |
| | APELLGGPSVFLFPPKPK | PAPIEKTISKAKGQPREPQVYTLPPS | SGLYSLSSVVTVPS |
| | DTLMISRTPEVTCVVVDV | REEMTKNQVSLTCLVKGFYPSDIAVE | SSLGTQTYICNVNH |
| | SHEDPEVKFNWYVDGVE | WESNGQPENNYKTTPPVLDSDGSFF | KPSNTKVDKRVEP |
| | VHNAKTKPREEQYNSTY | LYSKLTVDKSRWQQGNVFSCSVMH | KSCDKTHSGSSSS |
| | RVVSVLTVLHQDWLNGK | EALHNHYTQKSLSLSPGSTGSQVQL | GSSSSGSSSSTKK |
| | EYKCKVSNKALPAPIEKTI | VQSGAEVKKPGSSVKVSCKASGYTF | TQLQLEHLLLDLQM |
| | SKAKGQPREPQVYTLPP | SDYVINWVRQAPGQGLEWMGEIYPG | ILNGINNYKNPKLT |
| | SREEMTKNQVSLTCLVK | SGTNYYNEKFKAKATITADKSTSTAY | AMLTKKFYMPKKA |
| | GFYPSDIAVEWESNGQP | MELSSLRSEDTAVYYCARRGRYGLY | TELKHLQCLEEELK |
| | ENNYKTTPPVLDSDGSF | AMDYWGQGTTVTVSSRTVAAPSVFI | PLEEVLNLAQSKNF |
| | FLYSKLTVDKSRWQQGN | FPPSDEQLKSGTASVVCLLNNFYPRE | HLRPRDLISNINVIV |
| | VFSCSVMHEALHNHYTQ | AKVQWKVDNALQSGNSQESVTEQD | LELKGSETTFMCE |
| | KSLSLSPGK | SKDSTYSLSSTLTLSKADYEKHKVYA | YADETATIVEFLNR |
| | (SEQ ID NO: 209) | CEVTHQGLSSPVTKSFNRGEC | WITFAQSIISTLT |
| | | (SEQ ID NO: 210) | (SEQ ID NO: 211) |
| CD20- | MSVPTQVLGLLLLWLTDA | MEWSWVFLFFLSVTTGVHSEVQLVE | MSVPTQVLGLLLL |
| T6- | RCEIVLTQSPATLSLSPG | SGGGLVQPDRSLRLSCAASGFTFHD | WLTDARCDIQMTQ |
| NKp46-1- | ERATLSCRASQSVSSYL | YAMHWVRQAPGKGLEWVSTISWNS | SPSSLSASVGDRV |
| IL2v2A | AWYQQKPGQAPRLLIYD | GTIGYADSVKGRFTISRDNAKNSLYL | TITCRASQDISNYL |
| | ASNRATGIPARFSGSGS | QMNSLRAEDTALYYCAKDIQYGNYY | NWYQQKPGKAPKL |
| | GTDFTLTISSLEPEDFAV | YGMDVWGQGTTVTVSSASTKGPSV | LIYYTSRLHSGVPS |
| | YYCQQRSNWPITFGQGT | FPLAPSSKSTSGGTAALGCLVKDYFP | RFSGSGSGTDFTF |
| | RLEIKRTVAAPSVFIFPPS | EPVTVSWNSGALTSGVHTFPAVLQS | TISSLQPEDIATYFC |
| | DEQLKSGTASVVCLLNN | SGLYSLSSVVTVPSSSLGTQTYICNV | QQGNTRPWTFGG |
| | FYPREAKVQWKVDNALQ | NHKPSNTKVDKRVEPKSCDKTHTCP | GTKVEIKASTKGPS |
| | SGNSQESVTEQDSKDST | PCPAPELLGGPSVFLFPPKPKDTLMI | VFPLAPSSKSTSG |
| | YSLSSTLTLSKADYEKHK | SRTPEVTCVVVDVSHEDPEVKFNWY | GTAALGCLVKDYF |
| | VYACEVTHQGLSSPVTK | VDGVEVHNAKTKPREEQYSSTYRVV | PEPVTVSWNSGAL |
| | SFNRGECDKTHTCPPCP | SVLTVLHQDWLNGKEYKCKVSNKAL | TSGVHTFPAVLQS |
| | APELLGGPSVFLFPPKPK | PAPIEKTISKAKGQPREPQVYTLPPS | SGLYSLSSVVTVPS |
| | DTLMISRTPEVTCVVVDV | REEMTKNQVSLTCLVKGFYPSDIAVE | SSLGTQTYICNVNH |
| | SHEDPEVKFNWYVDGVE | WESNGQPENNYKTTPPVLDSDGSFF | KPSNTKVDKRVEP |
| | VHNAKTKPREEQYSSTY | LYSKLTVDKSRWQQGNVFSCSVMH | KSCDKTHSGSSSS |
| | RVVSVLTVLHQDWLNGK | EALHNHYTQKSLSLSPGSTGSQVQL | GSSSSGSSSSTKK |
| | EYKCKVSNKALPAPIEKTI | VQSGAEVKKPGSSVKVSCKASGYTF | TQLQLEHLLLDLQM |
| | SKAKGQPREPQVYTLPP | SDYVINWVRQAPGQGLEWMGEIYPG | ILNGINNYKNPKLT |
| | SREEMTKNQVSLTCLVK | SGTNYYNEKFKAKATITADKSTSTAY | AMLTKKFYMPKKA |
| | GFYPSDIAVEWESNGQP | MELSSLRSEDTAVYYCARRGRYGLY | TELKHLQCLEEELK |
| | ENNYKTTPPVLDSDGSF | AMDYWGQGTTVTVSSRTVAAPSVFI | PLEEVLNLAQSKNF |
| | FLYSKLTVDKSRWQQGN | FPPSDEQLKSGTASVVCLLNNFYPRE | HLRPRDLISNINVIV |
| | VFSCSVMHEALHNHYTQ | AKVQWKVDNALQSGNSQESVTEQD | LELKGSETTFMCE |
| | KSLSLSPGK | SKDSTYSLSSTLTLSKADYEKHKVYA | YADETATIVEFLNR |
| | (SEQ ID NO: 212) | CEVTHQGLSSPVTKSFNRGEC | WITFAQSIISTLT |
| | | (SEQ ID NO: 213) | (SEQ ID NO: 211) |
| CD20- | MSVPTQVLGLLLLWLTDA | MEWSWVFLFFLSVTTGVHSEVQLVE | |
| T13- | RCEIVLTQSPATLSLSPG | SGGGLVQPDRSLRLSCAASGFTFHD | |
| NKp46-1- | ERATLSCRASQSVSSYL | YAMHWVRQAPGKGLEWVSTISWNS | |
| IL2v2A | AWYQQKPGQAPRLLIYD | GTIGYADSVKGRFTISRDNAKNSLYL | |
| | ASNRATGIPARFSGSGS | QMNSLRAEDTALYYCAKDIQYGNYY | |
| | GTDFTLTISSLEPEDFAV | YGMDVWGQGTTVTVSSASTKGPSV | |
| | YYCQQRSNWPITFGQGT | FPLAPSSKSTSGGTAALGCLVKDYFP | |
| | RLEIKRTVAAPSVFIFPPS | EPVTVSWNSGALTSGVHTFPAVLQS | |
| | DEQLKSGTASVVCLLNN | SGLYSLSSVVTVPSSSLGTQTYICNV | |
| | FYPREAKVQWKVDNALQ | NHKPSNTKVDKRVEPKSCDKTHTCP | |
| | SGNSQESVTEQDSKDST | PCPAPELLGGPSVFLFPPKPKDTLMI | |
| | YSLSSTLTLSKADYEKHK | SRTPEVTCVVVDVSHEDPEVKFNWY | |
| | VYACEVTHQGLSSPVTK | VDGVEVHNAKTKPREEQYNSTYRVV | |
| | SFNRGECDKTHTCPPCP | SVLTVLHQDWLNGKEYKCKVSNKAL | |
| | APELLGGPSVFLFPPKPK | PAPIEKTISKAKGQPREPQVYTLPPS | |
| | DTLMISRTPEVTCVVVDV | REEMTKNQVSLTCLVKGFYPSDIAVE | |
| | SHEDPEVKFNWYVDGVE | WESNGQPENNYKTTPPVLDSDGSFF | |
| | VHNAKTKPREEQYNSTY | LYSKLTVDKSRWQQGNVFSCSVMH | |
| | RVVSVLTVLHQDWLNGK | EALHNHYTQKSLSLSPGSTGSQVQL | |
| | EYKCKVSNKALPAPIEKTI | VQSGAEVKKPGSSVKVSCKASGYTF | |
| | SKAKGQPREPQVYTLPP | SDYVINWVRQAPGQGLEWMGEIYPG | |
| | SREEMTKNQVSLTCLVK | SGTNYYNEKFKAKATITADKSTSTAY | |
| | GFYPSDIAVEWESNGQP | MELSSLRSEDTAVYYCARRGRYGLY | |
| | ENNYKTTPPVLDSDGSF | AMDYWGQGTTVTVSSVEGGSGGSG | |
| | FLYSKLTVDKSRWQQGN | GSGGSGGVDDIQMTQSPSSLSASVG | |
| | VFSCSVMHEALHNHYTQ | DRVTITCRASQDISNYLNWYQQKPG | |
| | KSLSLSPGK | KAPKLLIYYTSRLHSGVPSRFSGSGS | |
| | (SEQ ID NO: 209) | GTDFTFTISSLQPEDIATYFCQQGNT | |
| | | RPWTFGGGTKVEIKGSSSSGSSSSG | |
| | | SSSSTKKTQLQLEHLLLDLQMILNGIN | |
| | | NYKNPKLTAMLTKKFYMPKKATELKH | |
| | | LQCLEEELKPLEEVLNLAQSKNFHLR | |
| | | PRDLISNINVIVLELKGSETTFMCEYA | |
| | | DETATIVEFLNRWITFAQSIISTLT | |
| | | (SEQ ID NO: 214) | |
| | DIVMTQTPLSLPVTPGEP | QVQLVQSGAEVKKPGSSVKVSCKAS | DIQMTQSPSSLSAS |
| CD20- | ASISCRSSKSLLHSNGITY | GYAFSYSWINWVRQAPGQGLEWMG | VGDRVTITCRASQ |
| F5- | LYWYLQKPGQSPQLLIY | RIFPGDGDTDYNGKFKGRVTITADKS | DISNYLNWYQQKP |
| NKp46-1 | QMSNLVSGVPDRFSGSG | TSTAYMELSSLRSEDTAVYYCARNVF | GKAPKLLIYYTSRL |
| | SGTDFTLKISRVEAEDVG | DGYWLVYWGQGTLVTVSSASTKGP | HSGVPSRFSGSGS |
| | VYYCAQNLELPYTFGGG | SVFPLAPSSKSTSGGTAALGCLVKDY | GTDFTFTISSLQPE |
| | TKVEIKRTVAAPSVFIFPP | FPEPVTVSWNSGALTSGVHTFPAVL | DIATYFCQQGNTR |
| | SDEQLKSGTASVVCLLN | QSSGLYSLSSVVTVPSSSLGTQTYIC | PWTFGGGTKVEIK |
| | NFYPREAKVQWKVDNAL | NVNHKPSNTKVDKRVEPKSCDKTHT | ASTKGPSVFPLAPS |
| | QSGNSQESVTEQDSKDS | CPPCPAPELLGGPSVFLFPPKPKDTL | SKSTSGGTAALGC |
| | TYSLSSTLTLSKADYEKH | MISRTPEVTCVVVDVSHEDPEVKFN | LVKDYFPEPVTVS |
| | KVYACEVTHQGLSSPVT | WYVDGVEVHNAKTKPREEQYNSTY | WNSGALTSGVHTF |
| | KSFNRGECDKTHTCPPC | RVVSVLTVLHQDWLNGKEYKCKVSN | PAVLQSSGLYSLSS |
| | PAPELLGGPSVFLFPPKP | KALPAPIEKTISKAKGQPREPQVYTLP | VVTVPSSSLGTQT |
| | KDTLMISRTPEVTCVVVD | PSREEMTKNQVSLTCLVKGFYPSDIA | YICNVNHKPSNTKV |
| | VSHEDPEVKFNWYVDGV | VEWESNGQPENNYKTTPPVLDSDGS | DKRVEPKSCDKTH |
| | EVHNAKTKPREEQYNST | FFLYSKLTVDKSRWQQGNVFSCSVM | S |
| | YRVVSVLTVLHQDWLNG | HEALHNHYTQKSLSLSPGSTGSQVQ | (SEQ ID NO: 217) |
| | KEYKCKVSNKALPAPIEK | LVQSGAEVKKPGSSVKVSCKASGYT | |
| | TISKAKGQPREPQVYTLP | FSDYVINWVRQAPGQGLEWMGEIYP | |
| | PSREEMTKNQVSLTCLV | GSGTNYYNEKFKAKATITADKSTSTA | |
| | KGFYPSDIAVEWESNGQ | YMELSSLRSEDTAVYYCARRGRYGL | |
| | PENNYKTTPPVLDSDGS | YAMDYWGQGTTVTVSSRTVAAPSVF | |
| | FFLYSKLTVDKSRWQQG | IFPPSDEQLKSGTASVVCLLNNFYPR | |
| | NVFSCSVMHEALHNHYT | EAKVQWKVDNALQSGNSQESVTEQ | |
| | QKSLSLSPGK | DSKDSTYSLSSTLTLSKADYEKHKVY | |
| | (SEQ ID NO: 215) | ACEVTHQGLSSPVTKSFNRGEC | |
| | | (SEQ ID NO: 216) | |
| CD20- | MSVPTQVLGLLLLWLTDA | MEWSWVFLFFLSVTTGVHSEVQLVE | MSVPTQVLGLLLL |
| T5- | RCEIVLTQSPATLSLSPG | SGGGLVQPDRSLRLSCAASGFTFHD | WLTDARCDIQMTQ |
| NKp46-1- | ERATLSCRASQSVSSYL | YAMHWVRQAPGKGLEWVSTISWNS | SPSSLSASVGDRV |
| IL-2v3 | AWYQQKPGQAPRLLIYD | GTIGYADSVKGRFTISRDNAKNSLYL | TITCRASQDISNYL |
| | ASNRATGIPARFSGSGS | QMNSLRAEDTALYYCAKDIQYGNYY | NWYQQKPGKAPKL |
| | GTDFTLTISSLEPEDFAV | YGMDVWGQGTTVTVSSASTKGPSV | LIYYTSRLHSGVPS |
| | YYCQQRSNWPITFGQGT | FPLAPSSKSTSGGTAALGCLVKDYFP | RFSGSGSGTDFTF |
| | RLEIKRTVAAPSVFIFPPS | EPVTVSWNSGALTSGVHTFPAVLQS | TISSLQPEDIATYFC |
| | DEQLKSGTASVVCLLNN | SGLYSLSSVVTVPSSSLGTQTYICNV | QQGNTRPWTFGG |
| | FYPREAKVQWKVDNALQ | NHKPSNTKVDKRVEPKSCDKTHTCP | GTKVEIKASTKGPS |
| | SGNSQESVTEQDSKDST | PCPAPELLGGPSVFLFPPKPKDTLMI | VFPLAPSSKSTSG |
| | YSLSSTLTLSKADYEKHK | SRTPEVTCVVVDVSHEDPEVKFNWY | GTAALGCLVKDYF |
| | VYACEVTHQGLSSPVTK | VDGVEVHNAKTKPREEQYNSTYRVV | PEPVTVSWNSGAL |
| | SFNRGECDKTHTCPPCP | SVLTVLHQDWLNGKEYKCKVSNKAL | TSGVHTFPAVLQS |
| | APELLGGPSVFLFPPKPK | PAPIEKTISKAKGQPREPQVYTLPPS | SGLYSLSSVVTVPS |
| | DTLMISRTPEVTCVVVDV | REEMTKNQVSLTCLVKGFYPSDIAVE | SSLGTQTYICNVNH |
| | SHEDPEVKFNWYVDGVE | WESNGQPENNYKTTPPVLDSDGSFF | KPSNTKVDKRVEP |
| | VHNAKTKPREEQYNSTY | LYSKLTVDKSRWQQGNVFSCSVMH | KSCDKTHSGSSSS |
| | RVVSVLTVLHQDWLNGK | EALHNHYTQKSLSLSPGSTGSQVQL | GSSSSGSSSSTKK |
| | EYKCKVSNKALPAPIEKTI | VQSGAEVKKPGSSVKVSCKASGYTF | TQLQLEHLLLDLQM |
| | SKAKGQPREPQVYTLPP | SDYVINWVRQAPGQGLEWMGEIYPG | ILNGINNYKNPKLT |
| | SREEMTKNQVSLTCLVK | SGTNYYNEKFKAKATITADKSTSTAY | AMLAKKFYMPKKA |
| | GFYPSDIAVEWESNGQP | MELSSLRSEDTAVYYCARRGRYGLY | TELKHLQCLEEELK |
| | ENNYKTTPPVLDSDGSF | AMDYWGQGTTVTVSSRTVAAPSVFI | PLEEVLNLAQSKNF |
| | FLYSKLTVDKSRWQQGN | FPPSDEQLKSGTASVVCLLNNFYPRE | HLRPRDLISNINVIV |
| | VFSCSVMHEALHNHYTQ | AKVQWKVDNALQSGNSQESVTEQD | LELKGSETTFMCE |
| | KSLSLSPGK | SKDSTYSLSSTLTLSKADYEKHKVYA | YADETATIVEFLNR |
| | (SEQ ID NO: 209) | CEVTHQGLSSPVTKSFNRGEC | WITFCQSIISTLT |
| | | (SEQ ID NO: 210) | (SEQ ID NO: 218) |
| CD20- | MSVPTQVLGLLLLWLTDA | MEWSWVFLFFLSVTTGVHSEVQLVE | MSVPTQVLGLLLL |
| T5- | RCEIVLTQSPATLSLSPG | SGGGLVQPDRSLRLSCAASGFTFHD | WLTDARCDIQMTQ |
| NKp46-1- | ERATLSCRASQSVSSYL | YAMHWVRQAPGKGLEWVSTISWNS | SPSSLSASVGDRV |
| IL-2pWT | AWYQQKPGQAPRLLIYD | GTIGYADSVKGRFTISRDNAKNSLYL | TITCRASQDISNYL |
| | ASNRATGIPARFSGSGS | QMNSLRAEDTALYYCAKDIQYGNYY | NWYQQKPGKAPKL |
| | GTDFTLTISSLEPEDFAV | YGMDVWGQGTTVTVSSASTKGPSV | LIYYTSRLHSGVPS |
| | YYCQQRSNWPITFGQGT | FPLAPSSKSTSGGTAALGCLVKDYFP | RFSGSGSGTDFTF |
| | RLEIKRTVAAPSVFIFPPS | EPVTVSWNSGALTSGVHTFPAVLQS | TISSLQPEDIATYFC |
| | DEQLKSGTASVVCLLNN | SGLYSLSSVVTVPSSSLGTQTYICNV | QQGNTRPWTFGG |
| | FYPREAKVQWKVDNALQ | NHKPSNTKVDKRVEPKSCDKTHTCP | GTKVEIKASTKGPS |
| | SGNSQESVTEQDSKDST | PCPAPELLGGPSVFLFPPKPKDTLMI | VFPLAPSSKSTSG |
| | YSLSSTLTLSKADYEKHK | SRTPEVTCVVVDVSHEDPEVKFNWY | GTAALGCLVKDYF |
| | VYACEVTHQGLSSPVTK | VDGVEVHNAKTKPREEQYNSTYRVV | PEPVTVSWNSGAL |
| | SFNRGECDKTHTCPPCP | SVLTVLHQDWLNGKEYKCKVSNKAL | TSGVHTFPAVLQS |
| | APELLGGPSVFLFPPKPK | PAPIEKTISKAKGQPREPQVYTLPPS | SGLYSLSSVVTVPS |
| | DTLMISRTPEVTCVVVDV | REEMTKNQVSLTCLVKGFYPSDIAVE | SSLGTQTYICNVNH |
| | SHEDPEVKFNWYVDGVE | WESNGQPENNYKTTPPVLDSDGSFF | KPSNTKVDKRVEP |
| | VHNAKTKPREEQYNSTY | LYSKLTVDKSRWQQGNVFSCSVMH | KSCDKTHSGSSSS |
| | RVVSVLTVLHQDWLNGK | EALHNHYTQKSLSLSPGSTGSQVQL | GSSSSGSSSSTKK |
| | EYKCKVSNKALPAPIEKTI | VQSGAEVKKPGSSVKVSCKASGYTF | TQLQLEHLLLDLQM |
| | SKAKGQPREPQVYTLPP | SDYVINWVRQAPGQGLEWMGEIYPG | ILNGINNYKNPKLT |
| | SREEMTKNQVSLTCLVK | SGTNYYNEKFKAKATITADKSTSTAY | RMLTFKFYMPKKA |
| | GFYPSDIAVEWESNGQP | MELSSLRSEDTAVYYCARRGRYGLY | TELKHLQCLEEELK |
| | ENNYKTTPPVLDSDGSF | AMDYWGQGTTVTVSSRTVAAPSVFI | PLEEVLNLAQSKNF |
| | FLYSKLTVDKSRWQQGN | FPPSDEQLKSGTASVVCLLNNFYPRE | HLRPRDLISNINVIV |
| | VFSCSVMHEALHNHYTQ | AKVQWKVDNALQSGNSQESVTEQD | LELKGSETTFMCE |
| | KSLSLSPGK | SKDSTYSLSSTLTLSKADYEKHKVYA | YADETATIVEFLNR |
| | (SEQ ID NO: 209) | CEVTHQGLSSPVTKSFNRGEC | WITFCQSIISTLT) |
| | | (SEQ ID NO: 210) | (SEQ ID NO: 219) |
| CD20- | DIVMTQTPLSLPVTPGEP | QVQLVQSGAEVKKPGSSVKVSCKAS | EIVLTQSPGTLSLS |
| T5-IC- | ASISCRSSKSLLHSNGITY | GYAFSYSWINWVRQAPGQGLEWMG | PGERATLSCRASQ |
| IL2v | LYWYLQKPGQSPQLLIY | RIFPGDGDTDYNGKFKGRVTITADKS | SVSSSYLAWYQQK |
| | QMSNLVSGVPDRFSGSG | TSTAYMELSSLRSEDTAVYYCARNVF | PGQAPRLLIYGASS |
| | SGTDFTLKISRVEAEDVG | DGYWLVYWGQGTLVTVSSASTKGP | RATGIPDRFSGSG |
| | VYYCAQNLELPYTFGGG | SVFPLAPSSKSTSGGTAALGCLVKDY | SGTDFTLTISRLEP |
| | TKVEIKRTVAAPSVFIFPP | FPEPVTVSWNSGALTSGVHTFPAVL | EDFAVYYCQQYGS |
| | SDEQLKSGTASVVCLLN | QSSGLYSLSSWVTVPSSSLGTQTYIC | STWTFGQGTKVEI |
| | NFYPREAKVQWKVDNAL | NVNHKPSNTKVDKRVEPKSCDKTHT | KASTKGPSVFPLAP |
| | QSGNSQESVTEQDSKDS | CPPCPAPELLGGPSVFLFPPKPKDTL | SSKSTSGGTAALG |
| | TYSLSSTLTLSKADYEKH | MISRTPEVTCVVVDVSHEDPEVKFN | CLVKDYFPEPVTVS |
| | KVYACEVTHQGLSSPVT | WYVDGVEVHNAKTKPREEQYNSTY | WNSGALTSGVHTF |
| | KSFNRGECDKTHTCPPC | RVVSVLTVLHQDWLNGKEYKCKVSN | PAVLQSSGLYSLSS |
| | PAPELLGGPSVFLFPPKP | KALPAPIEKTISKAKGQPREPQVYTLP | VVTVPSSSLGTQT |
| | KDTLMISRTPEVTCVVVD | PSREEMTKNQVSLTCLVKGFYPSDIA | YICNVNHKPSNTKV |
| | VSHEDPEVKFNWYVDGV | VEWESNGQPENNYKTTPPVLDSDGS | DKRVEPKSCDKTH |
| | EVHNAKTKPREEQYNST | FFLYSKLTVDKSRWQQGNVFSCSVM | SGSSSSGSSSSGS |
| | YRVVSVLTVLHQDWLNG | HEALHNHYTQKSLSLSPGSTGSEVQ | SSSAPASSSTKKT |
| | KEYKCKVSNKALPAPIEK | LVQSGAEVKKSGESLKISCKGSGYSF | QLQLEHLLLDLQMI |
| | TISKAKGQPREPQVYTLP | TSYWIGWVRQMPGKGLEWMGIFYP | LNGINNYKNPKLTR |
| | PSREEMTKNQVSLTCLV | GDSSTRYSPSFQGQVTISADKSVNT | MLTAKFAMPKKAT |
| | KGFYPSDIAVEWESNGQ | AYLQWSSLKASDTAMYYCARRRNW | ELKHLQCLEEELKP |
| | PENNYKTTPPVLDSDGS | GNAFDIWGQGTMVTVSSRTVAAPSV | LEEVLNGAQSKNF |
| | FFLYSKLTVDKSRWQQG | FIFPPSDEQLKSGTASVVCLLNNFYP | HLRPRDLISNINVIV |
| | NVFSCSVMHEALHNHYT | REAKVQWKVDNALQSGNSQESVTE | LELKGSETTFMCE |
| | QKSLSLSPGK | QDSKDSTYSLSSTLTLSKADYEKHKV | YADETATIVEFLNR |
| | (SEQ ID NO: 220) | YACEVTHQGLSSPVTKSFNRGEC | WITFAQSIISTLT |
| | | (SEQ ID NO: 221) | (SEQ ID NO: 222) |
| CD20- | DIVMTQTPLSLPVTPGEP | QVQLVQSGAEVKKPGSSVKVSCKAS | DIQMTQSPSSLSAS |
| T6- | ASISCRSSKSLLHSNGITY | GYAFSYSWINWVRQAPGQGLEWMG | VGDRVTITCRASQ |
| NKp46- | LYWYLQKPGQSPQLLIY | RIFPGDGDTDYNGKFKGRVTITADKS | DISNYLNWYQQKP |
| IL2v3 | QMSNLVSGVPDRFSGSG | TSTAYMELSSLRSEDTAVYYCARNVF | GKAPKLLIYYTSRL |
| | SGTDFTLKISRVEAEDVG | DGYWLVYWGQGTLVTVSSASTKGP | HSGVPSRFSGSGS |
| | VYYCAQNLELPYTFGGG | SVFPLAPSSKSTSGGTAALGCLVKDY | GTDFTFTISSLQPE |
| | TKVEIKRTVAAPSVFIFPP | FPEPVTVSWNSGALTSGVHTFPAVL | DIATYFCQQGNTR |
| | SDEQLKSGTASVVCLLN | QSSGLYSLSSWVTVPSSSLGTQTYIC | PWTFGGGTKVEIK |
| | NFYPREAKVQWKVDNAL | NVNHKPSNTKVDKRVEPKSCDKTHT | ASTKGPSVFPLAPS |
| | QSGNSQESVTEQDSKDS | CPPCPAPELLGGPSVFLFPPKPKDTL | SKSTSGGTAALGC |
| | TYSLSSTLTLSKADYEKH | MISRTPEVTCVVVDVSHEDPEVKFN | LVKDYFPEPVTVS |
| | KVYACEVTHQGLSSPVT | WYVDGVEVHNAKTKPREEQYSSTYR | WNSGALTSGVHTF |
| | KSFNRGECDKTHTCPPC | VVSVLTVLHQDWLNGKEYKCKVSNK | PAVLQSSGLYSLSS |
| | PAPELLGGPSVFLFPPKP | ALPAPIEKTISKAKGQPREPQVYTLPP | VVTVPSSSLGTQT |
| | KDTLMISRTPEVTCVVVD | SREEMTKNQVSLTCLVKGFYPSDIAV | YICNVNHKPSNTKV |
| | VSHEDPEVKFNWYVDGV | EWESNGQPENNYKTTPPVLDSDGSF | DKRVEPKSCDKTH |
| | EVHNAKTKPREEQYSST | FLYSKLTVDKSRWQQGNVFSCSVMH | SGSSSSGSSSSGS |
| | YRVVSVLTVLHQDWLNG | EALHNHYTQKSLSLSPGSTGSQVQL | SSSTKKTQLQLEHL |
| | KEYKCKVSNKALPAPIEK | VQSGAEVKKPGSSVKVSCKASGYTF | LLDLQMILNGINNY |
| | TISKAKGQPREPQVYTLP | SDYVINWVRQAPGQGLEWMGEIYPG | KNPKLTAMLAKKFY |
| | PSREEMTKNQVSLTCLV | SGTNYYNEKFKAKATITADKSTSTAY | MPKKATELKHLQC |
| | KGFYPSDIAVEWESNGQ | MELSSLRSEDTAVYYCARRGRYGLY | LEEELKPLEEVLNL |
| | PENNYKTTPPVLDSDGS | AMDYWGQGTTVTVSSRTVAAPSVFI | AQSKNFHLRPRDLI |
| | FFLYSKLTVDKSRWQQG | FPPSDEQLKSGTASVVCLLNNFYPRE | SNINVIVLELKGSET |
| | NVFSCSVMHEALHNHYT | AKVQWKVDNALQSGNSQESVTEQD | TFMCEYADETATIV |
| | QKSLSLSPGK | SKDSTYSLSSTLTLSKADYEKHKVYA | EFLNRWITFCQSIIS |
| | (SEQ ID NO: 223) | CEVTHQGLSSPVTKSFNRGEC | TLT |
| | | (SEQ ID NO: 224) | (SEQ ID NO: 225) |
| IC-T5- | EIVLTQSPATLSLSPGER | EVQLVESGGGLVQPGRSLRLSCAAS | DIQMTQSPSSLSAS |
| NKp46-1- | ATLSCRASQSVRSYLAW | GFTFDNYAMHWVRQAPGKGLEWVS | VGDRVTITCRASQ |
| IL-2v | YQQKPGQAPRLLFSDAS | GISRSSGDIDYADSVKGRFTISRDNA | DISNYLNWYQQKP |
| | NRATGIPARFSGSGSGT | KNSLYLQMNSLRAEDTALYYCARGG | GKAPKLLIYYTSRL |
| | DFTLTISSLEPEDFAVYY | VGSFDTWGQGTMVTVSSASTKGPS | HSGVPSRFSGSGS |
| | CQQYRYSPRTFGQGTKV | VFPLAPSSKSTSGGTAALGCLVKDYF | GTDFTFTISSLQPE |
| | EIKRTVAAPSVFIFPPSDE | PEPVTVSWNSGALTSGVHTFPAVLQ | DIATYFCQQGNTR |
| | QLKSGTASVVCLLNNFYP | SSGLYSLSSVVTVPSSSLGTQTYICN | PWTFGGGTKVEIK |
| | REAKVQWKVDNALQSG | VNHKPSNTKVDKRVEPKSCDKTHTC | ASTKGPSVFPLAPS |
| | NSQESVTEQDSKDSTYS | PPCPAPELLGGPSVFLFPPKPKDTLM | SKSTSGGTAALGC |
| | LSSTLTLSKADYEKHKVY | ISRTPEVTCVVVDVSHEDPEVKFNW | LVKDYFPEPVTVS |
| | ACEVTHQGLSSPVTKSF | YVDGVEVHNAKTKPREEQYNSTYRV | WNSGALTSGVHTF |
| | NRGECDKTHTCPPCPAP | VSVLTVLHQDWLNGKEYKCKVSNKA | PAVLQSSGLYSLSS |
| | ELLGGPSVFLFPPKPKDT | LPAPIEKTISKAKGQPREPQVYTLPPS | VVTVPSSSLGTQT |
| | LMISRTPEVTCVVVDVSH | REEMTKNQVSLTCLVKGFYPSDIAVE | YICNVNHKPSNTKV |
| | EDPEVKFNWYVDGVEVH | WESNGQPENNYKTTPPVLDSDGSFF | DKRVEPKSCDKTH |
| | NAKTKPREEQYNSTYRV | LYSKLTVDKSRWQQGNVFSCSVMH | SGSSSSGSSSSAP |
| | VSVLTVLHQDWLNGKEY | EALHNHYTQKSLSLSPGSTGSQVQL | ASSSTKKTQLQLE |
| | KCKVSNKALPAPIEKTISK | VQSGAEVKKPGSSVKVSCKASGYTF | HLLLDLQMILNGIN |
| | AKGQPREPQVYTLPPSR | SDYVINWVRQAPGQGLEWMGEIYPG | NYKNPKLTRMLTA |
| | EEMTKNQVSLTCLVKGF | SGTNYYNEKFKAKATITADKSTSTAY | KFAMPKKATELKHL |
| | YPSDIAVEWESNGQPEN | MELSSLRSEDTAVYYCARRGRYGLY | QCLEEELKPLEEVL |
| | NYKTTPPVLDSDGSFFLY | AMDYWGQGTTVTVSSRTVAAPSVFI | NGAQSKNFHLRPR |
| | SKLTVDKSRWQQGNVFS | FPPSDEQLKSGTASVVCLLNNFYPRE | DLISNINVIVLELKG |
| | CSVMHEALHNHYTQKSL | AKVQWKVDNALQSGNSQESVTEQD | SETTFMCEYADET |
| | SLSPGK | SKDSTYSLSSTLTLSKADYEKHKVYA | ATIVEFLNRWITFA |
| | (SEQ ID NO: 226) | CEVTHQGLSSPVTKSFNRGEC | QSIISTLT |
| | | (SEQ ID NO: 227) | (SEQ ID NO: 228) |
| IC-T6-IC- | EIVLTQSPATLSLSPGER | EVQLVESGGGLVQPGRSLRLSCAAS | EIVLTQSPGTLSLS |
| IL2v | ATLSCRASQSVRSYLAW | GFTFDNYAMHWVRQAPGKGLEWVS | PGERATLSCRASQ |
| | YQQKPGQAPRLLFSDAS | GISRSSGDIDYADSVKGRFTISRDNA | SVSSSYLAWYQQK |
| | NRATGIPARFSGSGSGT | KNSLYLQMNSLRAEDTALYYCARGG | PGQAPRLLIYGASS |
| | DFTLTISSLEPEDFAVYY | VGSFDTWGQGTMVTVSSASTKGPS | RATGIPDRFSGSG |
| | CQQYRYSPRTFGQGTKV | VFPLAPSSKSTSGGTAALGCLVKDYF | SGTDFTLTISRLEP |
| | EIKRTVAAPSVFIFPPSDE | PEPVTVSWNSGALTSGVHTFPAVLQ | EDFAVYYCQQYGS |
| | QLKSGTASVVCLLNNFYP | SSGLYSLSSVVTVPSSSLGTQTYICN | STWTFGQGTKVEI |
| | REAKVQWKVDNALQSG | VNHKPSNTKVDKRVEPKSCDKTHTC | KASTKGPSVFPLAP |
| | NSQESVTEQDSKDSTYS | PPCPAPELLGGPSVFLFPPKPKDTLM | SSKSTSGGTAALG |
| | LSSTLTLSKADYEKHKVY | ISRTPEVTCVVVDVSHEDPEVKFNW | CLVKDYFPEPVTVS |
| | ACEVTHQGLSSPVTKSF | YVDGVEVHNAKTKPREEQYSSTYRV | WNSGALTSGVHTF |
| | NRGECDKTHTCPPCPAP | VSVLTVLHQDWLNGKEYKCKVSNKA | PAVLQSSGLYSLSS |
| | ELLGGPSVFLFPPKPKDT | LPAPIEKTISKAKGQPREPQVYTLPPS | VVTVPSSSLGTQT |
| | LMISRTPEVTCVVVDVSH | REEMTKNQVSLTCLVKGFYPSDIAVE | YICNVNHKPSNTKV |
| | EDPEVKFNWYVDGVEVH | WESNGQPENNYKTTPPVLDSDGSFF | DKRVEPKSCDKTH |
| | NAKTKPREEQYSSTYRV | LYSKLTVDKSRWQQGNVFSCSVMH | SGSSSSGSSSSAP |
| | VSVLTVLHQDWLNGKEY | EALHNHYTQKSLSLSPGSTGSEVQL | ASSSTKKTQLQLE |
| | KCKVSNKALPAPIEKTISK | VQSGAEVKKSGESLKISCKGSGYSFT | HLLLDLQMILNGIN |
| | AKGQPREPQVYTLPPSR | SYWIGWVRQMPGKGLEWMGIFYPG | NYKNPKLTRMLTA |
| | EEMTKNQVSLTCLVKGF | DSSTRYSPSFQGQVTISADKSVNTAY | KFAMPKKATELKHL |
| | YPSDIAVEWESNGQPEN | LQWSSLKASDTAMYYCARRRNWGN | QCLEEELKPLEEVL |
| | NYKTTPPVLDSDGSFFLY | AFDIWGQGTMVTVSSRTVAAPSVFIF | NGAQSKNFHLRPR |
| | SKLTVDKSRWQQGNVFS | PPSDEQLKSGTASWVCLLNNFYPRE | DLISNINVIVLELKG |
| | CSVMHEALHNHYTQKSL | AKVQWKVDNALQSGNSQESVTEQD | SETTFMCEYADET |
| | SLSPGK | SKDSTYSLSSTLTLSKADYEKHKVYA | ATIVEFLNRWITFA |
| | (SEQ ID NO: 229) | CEVTHQGLSSPVTKSFNRGEC | QSIISTLT |
| | | (SEQ ID NO: 230) | (SEQ ID NO: 231) |
| CD20- | DIVMTQTPLSLPVTPGEP | QVQLVQSGAEVKKPGSSVKVSCKAS | DIQMTQSPSSLSAS |
| F25- | ASISCRSSKSLLHSNGITY | GYAFSYSWINWVRQAPGQGLEWMG | VGDRVTITCRASQ |
| NKp46-1 | LYWYLQKPGQSPQLLIY | RIFPGDGDTDYNGKFKGRVTITADKS | DISNYLNWYQQKP |
| | QMSNLVSGVPDRFSGSG | TSTAYMELSSLRSEDTAVYYCARNVF | GKAPKLLIYYTSRL |
| | SGTDFTLKISRVEAEDVG | DGYWLVYWGQGTLVTVSSASTKGP | HSGVPSRFSGSGS |
| | VYYCAQNLELPYTFGGG | SVFPLAPSSKSTSGGTAALGCLVKDY | GTDFTFTISSLQPE |
| | TKVEIKRTVAAPSVFIFPP | FPEPVTVSWNSGALTSGVHTFPAVL | DIATYFCQQGNTR |
| | SDEQLKSGTASVVCLLN | QSSGLYSLSSWVTVPSSSLGTQTYIC | PWTFGGGTKVEIK |
| | NFYPREAKVQWKVDNAL | NVNHKPSNTKVDKRVEPKSCDKTHT | RTVAAPSVFIFPPS |
| | QSGNSQESVTEQDSKDS | CPPCPAPELLGGPSVFLFPPKPKDTL | DEQLKSGTASVVC |
| | TYSLSSTLTLSKADYEKH | MISRTPEVTCVVVDVSHEDPEVKFN | LLNNFYPREAKVQ |
| | KVYACEVTHQGLSSPVT | WYVDGVEVHNAKTKPREEQYNSTY | WKVDNALQSGNS |
| | KSFNRGECDKTHTCPPC | RVVSVLTVLHQDWLNGKEYKCKVSN | QESVTEQDSKDST |
| | PAPELLGGPSVFLFPPKP | KALPAPIEKTISKAKGQPREPQVYTLP | YSLSSTLTLSKADY |
| | KDTLMISRTPEVTCVVVD | PSREEMTKNQVSLTCLVKGFYPSDIA | EKHKVYACEVTHQ |
| | VSHEDPEVKFNWYVDGV | VEWESNGQPENNYKTTPPVLDSDGS | GLSSPVTKSFNRG |
| | EVHNAKTKPREEQYNST | FFLYSKLTVDKSRWQQGNVFSCSVM | EC (SEQ ID NO: |
| | YRVVSVLTVLHQDWLNG | HEALHNHYTQKSLSLSPGSTGSQVQ | 236) |
| | KEYKCKVSNKALPAPIEK | LVQSGAEVKKPGSSVKVSCKASGYT | |
| | TISKAKGQPREPQVYTLP | FSDYVINWVRQAPGQGLEWMGEIYP | |
| | PSREEMTKNQVSLTCLV | GSGTNYYNEKFKAKATITADKSTSTA | |
| | KGFYPSDIAVEWESNGQ | YMELSSLRSEDTAVYYCARRGRYGL | |
| | PENNYKTTPPVLDSDGS | YAMDYWGQGTTVTVSSASTKGPSVF | |
| | FFLYSKLTVDKSRWQQG | PLAPSSKSTSGGTAALGCLVKDYFPE | |
| | NVFSCSVMHEALHNHYT | PVTVSWNSGALTSGVHTFPAVLQSS | |
| | QKSLSLSPGK (SEQ ID | GLYSLSSVVTVPSSSLGTQTYICNVN | |
| | NO: 234) | HKPSNTKVDKRVEPKSCDKTHS | |
| | | (SEQ ID NO: 235) | |
| IC-F25- | EIVLTQSPATLSLSPGER | EVQLVESGGGLVQPGRSLRLSCAAS | DIQMTQSPSSLSAS |
| NKp46-1 | ATLSCRASQSVRSYLAW | GFTFDNYAMHWVRQAPGKGLEWVS | VGDRVTITCRASQ |
| | YQQKPGQAPRLLFSDAS | GISRSSGDIDYADSVKGRFTISRDNA | DISNYLNWYQQKP |
| | NRATGIPARFSGSGSGT | KNSLYLQMNSLRAEDTALYYCARGG | GKAPKLLIYYTSRL |
| | DFTLTISSLEPEDFAVYY | VGSFDTWGQGTMVTVSSASTKGPS | HSGVPSRFSGSGS |
| | CQQYRYSPRTFGQGTKV | VFPLAPSSKSTSGGTAALGCLVKDYF | GTDFTFTISSLQPE |
| | EIKRTVAAPSVFIFPPSDE | PEPVTVSWNSGALTSGVHTFPAVLQ | DIATYFCQQGNTR |
| | QLKSGTASVVCLLNNFYP | SSGLYSLSSVVTVPSSSLGTQTYICN | PWTFGGGTKVEIK |
| | REAKVQWKVDNALQSG | VNHKPSNTKVDKRVEPKSCDKTHTC | RTVAAPSVFIFPPS |
| | NSQESVTEQDSKDSTYS | PPCPAPELLGGPSVFLFPPKPKDTLM | DEQLKSGTASVVC |
| | LSSTLTLSKADYEKHKVY | ISRTPEVTCVVVDVSHEDPEVKFNW | LLNNFYPREAKVQ |
| | ACEVTHQGLSSPVTKSF | YVDGVEVHNAKTKPREEQYNSTYRV | WKVDNALQSGNS |
| | NRGECDKTHTCPPCPAP | VSVLTVLHQDWLNGKEYKCKVSNKA | QESVTEQDSKDST |
| | ELLGGPSVFLFPPKPKDT | LPAPIEKTISKAKGQPREPQVYTLPPS | YSLSSTLTLSKADY |
| | LMISRTPEVTCVVVDVSH | REEMTKNQVSLTCLVKGFYPSDIAVE | EKHKVYACEVTHQ |
| | EDPEVKFNWYVDGVEVH | WESNGQPENNYKTTPPVLDSDGSFF | GLSSPVTKSFNRG |
| | NAKTKPREEQYNSTYRV | LYSKLTVDKSRWQQGNVFSCSVMH | EC (SEQ ID NO: |
| | VSVLTVLHQDWLNGKEY | EALHNHYTQKSLSLSPGSTGSQVQL | 239) |
| | KCKVSNKALPAPIEKTISK | VQSGAEVKKPGSSVKVSCKASGYTF | |
| | AKGQPREPQVYTLPPSR | SDYVINWVRQAPGQGLEWMGEIYPG | |
| | EEMTKNQVSLTCLVKGF | SGTNYYNEKFKAKATITADKSTSTAY | |
| | YPSDIAVEWESNGQPEN | MELSSLRSEDTAVYYCARRGRYGLY | |
| | NYKTTPPVLDSDGSFFLY | AMDYWGQGTTVTVSSASTKGPSVFP | |
| | SKLTVDKSRWQQGNVFS | LAPSSKSTSGGTAALGCLVKDYFPEP | |
| | CSVMHEALHNHYTQKSL | VTVSWNSGALTSGVHTFPAVLQSSG | |
| | SLSPGK (SEQ ID NO: | LYSLSSVVTVPSSSLGTQTYICNVNH | |
| | 237) | KPSNTKVDKRVEPKSCDKTHS (SEQ | |
| | | ID NO: 238) | |

### Preparation of Genetically Modified NK Cells Expressing CAR Signaling Module:

DNA constructs encoding CAR signaling module as disclosed in Table F hereinafter were inserted within expression vectors and transfected into KHYG-1 cells (NK cells), Jurkat E6.1 (T cells), and HEK293F (as transfection control).

TABLE E
Materials

| In-Fusion HD Cloning kit | Takara | ST0345 |
|---|---|---|
| Stellar competent cells | Takara | ST0213 |
| Nucleobond Xtra Maxi plus EF 10 | Macherey-Nagel | 740426.10 |
| OPTI-MEM | Gibco | 31985-062 |
| Polyethylenimine (PEI) | Polyscience | 24765-1 |
| Trypton N1 | Organotechnie | 19553 |
| D(+)-Glucose monohydrate | Sigma | 49161-1kg |
| Valproic Acid | Merck | P4543-10G |
| Neon Transfection System 10 μL kit | Thermofisher Scientific | MPK1096 |
| PBS 1X | Gibco | 14190-094 |
| anti-hu CD335 (NKp46) PerCP-Cy5.5 | Becton Dickinson | 331920 |
| anti-hu CD335 (NKp46) PE | Becton Dickinson | 557991 |

Vector construction. The 6 DNA constructs referred to as SNK1 to SNK6 were synthetized by and resuspended in Tris HCl 10 mM. They were inserted into an expression vector using the In-Fusion HD Cloning kit. After ligation, Stellar-competent cells were transformed with thermic choc and plated on LB Agar Ampicillin plates. The next day colonies were screened by PCR and one colony was selected to amplified the final vector for each construct. Vectors were then extracted from bacteria using the Nucleobond Xtra Maxi plus EF 10 and sequenced.

HEK293F transfection. One day before transfection the HEK293F cell concentration was adjusted to 1 million per mL with a final volume of 20 mL. The day of transfection two mixes were realized: OptiMEM+Polyethylenimine and OptiMEM+33 μg of each vector. These two mixes were pooled and incubate for 15 min at room temperature. After incubation, the entire final mix was added to the cells and incubated for 3 days at 37° C., 5% CO₂. After one day of transfection three additives were added to the cells in order to improve protein expression: trypton, glucose and valproic acid.

Transfection of NK cells. KHYG-1 transfection was carried out using the Neon Transfection System 10 μL kit. KHYG-1 cells were previously counted and washed twice with sterile PBS 1×. Cell pellet was resuspended with the supplied buffer R and mixed with each vector (300 000 cells+700 ng of vector per point). Electroporation setting was 950V, 30 ms and 1 pulse. After electroporation the cells were spread in 24-well plate and incubated for few days at 37° C., 5% CO2.

The resulting NK cells transfected with NKp46-CD3zeta are referred to as SNK1 cells; NK cells transfected with NKp46-2B4-CD3zeta are referred to as SNK2 cells; NK cells transfected with NKp46-4-1 BB-CD3zeta are referred to as SNK3 cells; NK cells transfected with NKp46-CD28-4-1 BB-CD3zeta are referred to as SNK4 cells; NK cells transfected with NKp46-CD8a-CD28-4-1 BB-CD3zeta are referred to as SNK5 cells; NK cells transfected with NKp46-OX40-CD3zeta are referred to as SNK6 cells. See FIG. 5 for a schematic for the CAR module expressed by each NK cell tested.

Transfection of T cells. Jurkat E6.1 transfection was carried out using the Neon Transfection System 10 μL kit. Jurkat E6.1 cells were previously counted and washed twice with sterile PBS 1×. Cell pellet was resuspended with the supplied buffer R and mixed with each vector (300 000 cells+700 ng of vector per point). Electroporation setting was 1050V, 30 ms and 2 pulses. After electroporation the cells were spread in 24-well plate and incubated for few days at 37° C., 5% CO2.

The resulting T cells transfected with NKp46-2B4-CD3zeta are referred to as Jurkat SNK2 cells; T cells transfected with NKp46-CD28-4-1BB-CD3zeta are referred to as Jurkat SNK4 cells. See FIG. 5 for a schematic for the CAR module expressed by each T cell tested.

TABLE F

| Sample | Sequence |
|---|---|
| NKp46-CD3zeta | MSSTLPALLCVGLCLSQRISAQQQTLPKPFIWAEPHFMVPKEKQVTICCQGNY |
| | GAVEYQLHFEGSLFAVDRPKPPERINKVKFYIPDMNSRMAGQYSCIYRVGELW |
| | SEPSNLLDLVVTEMYDTPTLSVHPGPEVISGEKVTFYCRLDTATSMFLLLKEGR |
| | SSHVQRGYGKVQAEFPLGPVTTAHRGTYRCFGSYNNHAWSFPSEPVKLLVTG |
| | DIENTSLAPEDPTFPADTWGTYLLTTETGLQKDHALWDHTAQNLLRLCYLLDGI |
| | LFIYGVILTALFLRVKFSRSADAPAYQQGQNQLYNELNLGRREEYDVLDKRRGR |
| | DPEMGGKPQRRKNPQEGLYNELQKDKMAEAYSEIGMKGERRRGKGHDGLYQ |
| | GLSTATKDTYDALHMQALPPR (SEQ ID NO: 29) |
| NKp46-2B4- | MSSTLPALLCVGLCLSQRISAQQQTLPKPFIWAEPHFMVPKEKQVTICCQGNY |
| CD3zeta | GAVEYQLHFEGSLFAVDRPKPPERINKVKFYIPDMNSRMAGQYSCIYRVGELW |
| | SEPSNLLDLVVTEMYDTPTLSVHPGPEVISGEKVTFYCRLDTATSMFLLLKEGR |
| | SSHVQRGYGKVQAEFPLGPVTTAHRGTYRCFGSYNNHAWSFPSEPVKLLVTG |
| | DIENTSLAPEDPTFPADTWGTYLLTTETGLQKDHALWDHTAQNLLRFLVIIVILSA |
| | LFLGTLACFCVWRRKRKEKQSETSPKEFLTIYEDVKDLKTRRNHEQEQTFPGG |
| | GSTIYSMIQSQSSAPTSQEPAYTLYSLIQPSRKSGSRKRNHSPSFNSTIYEVIGK |
| | SQPKAQNPARLSRKELENFDVYSRVKFSRSADAPAYQQGQNQLYNELNLGRR |
| | EEYDVLDKRRGRDPEMGGKPQRRKNPQEGLYNELQKDKMAEAYSEIGMKGE |
| | RRRGKGHDGLYQGLSTATKDTYDALHMQALPPR (SEQ ID NO: 31) |
| NKp46-4-1BB- | MSSTLPALLCVGLCLSQRISAQQQTLPKPFIWAEPHFMVPKEKQVTICCQGNY |
| CD3zeta | GAVEYQLHFEGSLFAVDRPKPPERINKVKFYIPDMNSRMAGQYSCIYRVGELW |
| | SEPSNLLDLVVTEMYDTPTLSVHPGPEVISGEKVTFYCRLDTATSMFLLLKEGR |
| | SSHVQRGYGKVQAEFPLGPVTTAHRGTYRCFGSYNNHAWSFPSEPVKLLVTG |
| | DIENTSLAPEDPTFPADTWGTYLLTTETGLQKDHALWDHTAQNLLRIISFFLALT |
| | STALLFLLFFLTLRFSVVKRGRKKLLYIFKQPFMRPVQTTQEEDGCSCRFPEEE |
| | EGGCELRVKFSRSADAPAYQQGQNQLYNELNLGRREEYDVLDKRRGRDPEM |
| | GGKPQRRKNPQEGLYNELQKDKMAEAYSEIGMKGERRRGKGHDGLYQGLST |
| | ATKDTYDALHMQALPPR (SEQ ID NO: 41) |
| NKp46-CD28-4- | MSSTLPALLCVGLCLSQRISAQQQTLPKPFIWAEPHFMVPKEKQVTICCQGNY |
| 1BB-CD3zeta | GAVEYQLHFEGSLFAVDRPKPPERINKVKFYIPDMNSRMAGQYSCIYRVGELW |
| | SEPSNLLDLVVTEMYDTPTLSVHPGPEVISGEKVTFYCRLDTATSMFLLLKEGR |
| | SSHVQRGYGKVQAEFPLGPVTTAHRGTYRCFGSYNNHAWSFPSEPVKLLVTG |
| | DIENTSLAPEDPTFPADTWGTYLLTTETGLQKDHALWDHTAQNLLRFWVLVVV |
| | GGVLACYSLLVTVAFIIFWVRSKRSRLLHSDYMNMTPRRPGPTRKHYQPYAPP |
| | RDFAAYRSKRGRKKLLYIFKQPFMRPVQTTQEEDGCSCRFPEEEEGGCELRV |
| | KFSRSADAPAYQQGQNQLYNELNLGRREEYDVLDKRRGRDPEMGGKPQRRK |
| | NPQEGLYNELQKDKMAEAYSEIGMKGERRRGKGHDGLYQGLSTATKDTYDAL |
| | HMQALPPR (SEQ ID NO: 39) |
| NKp46-CD8a- | MSSTLPALLCVGLCLSQRISAQQQTLPKPFIWAEPHFMVPKEKQVTICCQGNY |
| CD28-4-1BB- | GAVEYQLHFEGSLFAVDRPKPPERINKVKFYIPDMNSRMAGQYSCIYRVGELW |
| CD3zeta | SEPSNLLDLVVTEMYDTPTLSVHPGPEVISGEKVTFYCRLDTATSMFLLLKEGR |
| | SSHVQRGYGKVQAEFPLGPVTTAHRGTYRCFGSYNNHAWSFPSEPVKLLVTG |
| | DIENTSLAPEDPTFPADTWGTYLLTTETGLQKDHALWDHTAQNLLRIYIWAPLA |
| | GTCGVLLLSLVITRSKRSRLLHSDYMNMTPRRPGPTRKHYQPYAPPRDFAAYR |
| | SKRGRKKLLYIFKQPFMRPVQTTQEEDGCSCRFPEEEEGGCELRVKFSRSAD |
| | APAYQQGQNQLYNELNLGRREEYDVLDKRRGRDPEMGGKPQRRKNPQEGLY |
| | NELQKDKMAEAYSEIGMKGERRRGKGHDGLYQGLSTATKDTYDALHMQALPP |
| | R (SEQ ID NO: 44) |
| NKp46-OX40- | MSSTLPALLCVGLCLSQRISAQQQTLPKPFIWAEPHFMVPKEKQVTICCQGNY |
| CD3zeta | GAVEYQLHFEGSLFAVDRPKPPERINKVKFYIPDMNSRMAGQYSCIYRVGELW |
| | SEPSNLLDLVVTEMYDTPTLSVHPGPEVISGEKVTFYCRLDTATSMFLLLKEGR |
| | SSHVQRGYGKVQAEFPLGPVTTAHRGTYRCFGSYNNHAWSFPSEPVKLLVTG |
| | DIENTSLAPEDPTFPADTWGTYLLTTETGLQKDHALWDHTAQNLLRVAAILGLG |
| | LVLGLLGPLAILLALYLLRRDQRLPPDAHKPPGGGSFRTPIQEEQADAHSTLAKI |
| | RVKFSRSADAPAYQQGQNQLYNELNLGRREEYDVLDKRRGRDPEMGGKPQR |
| | RKNPQEGLYNELQKDKMAEAYSEIGMKGERRRGKGHDGLYQGLSTATKDTYD |
| | ALHMQALPPR (SEQ ID NO: 45) |

### Example 2: CAR Potentiator Module Promotes Tumor Cell Killing in a Standard Vitro Cytotoxicity Assay at ET Ratio 10:1

In this experiment, CAR potentiator modules were assessed for their ability to induce killing of RAJI tumor cells by NK cells from two human donors at effector:target ratio of 10:1 in a standard 4-hour cytotoxicity assay using calcein release as readout.

The test proteins included in this experiment:
CD20-T5-NKp46-IL2v3: contains from topological N- to C-terminus, anti-CD20 VH/VL pair, Fc domain dimer that binds CD16, anti-NKp46-1 VH/VL pair, IL-2v3 which is a variant IL-2 including deletion of the three first residues of IL-2 mature protein (APT) and three mutation affecting CD25 binding (R38A, T41A, F42K).
CD20-T5-NKp46-IL2pWT: contains from topological N- to C-terminus, anti-CD20 VH/VL pair, Fc domain dimer that binds CD16, anti-NKp46-1 VH/VL pair, IL-2pWT which is wild-type human IL-2 polypeptide.
CD20-F5-NKp46: contains from topological N- to C-terminus, anti-CD20 VH/VL pair, Fc domain dimer that binds CD16, anti-NKp46-1 VH/VL pair.
CD20-T5-IC-IL2v: contains from topological N- to C-terminus, anti-CD20 VH/VL pair, Fc domain dimer that binds CD16, IC VH/VL pair, IL2v.
CD20-T6-NKp46-IL2v3: contains from topological N- to C-terminus, anti-CD20 VH/VL pair, Fc domain dimer mutated to abolish CD16 binding, anti-NKp46-1 VH/VL pair, IL-2v3.
IC-T5-NKp46-IL2v: contains from topological N- to C-terminus, IC VH/VL pair, Fc domain dimer that binds CD16, anti-NKp46-1 VH/VL pair, IL2v.
CD20-T5-NKp46-IL2v: contains from topological N- to C-terminus, anti-CD20 VH/VL pair, Fc domain dimer that binds CD16, anti-NKp46-1 VH/VL pair, IL2v.

Briefly, purified NK cells were rested overnight in complete medium. Purified NK Cells were then cocultured with Raji tumor cells previously loaded with calcein, in a 10 to 1 ratio. Cells were incubated with test proteins described above (doses from 66 nM to 0.0000006 nM) for 4 h at 37° C., 5.5% CO₂ in incubator. In order to provide a comparison to well-known anti-tumor antibodies, the protein were made to bind CD20 by incorporating anti-CD20 VH/VL pair from the FDA-approved humanized antibody GA101 (Obinutuzumab, Roche).

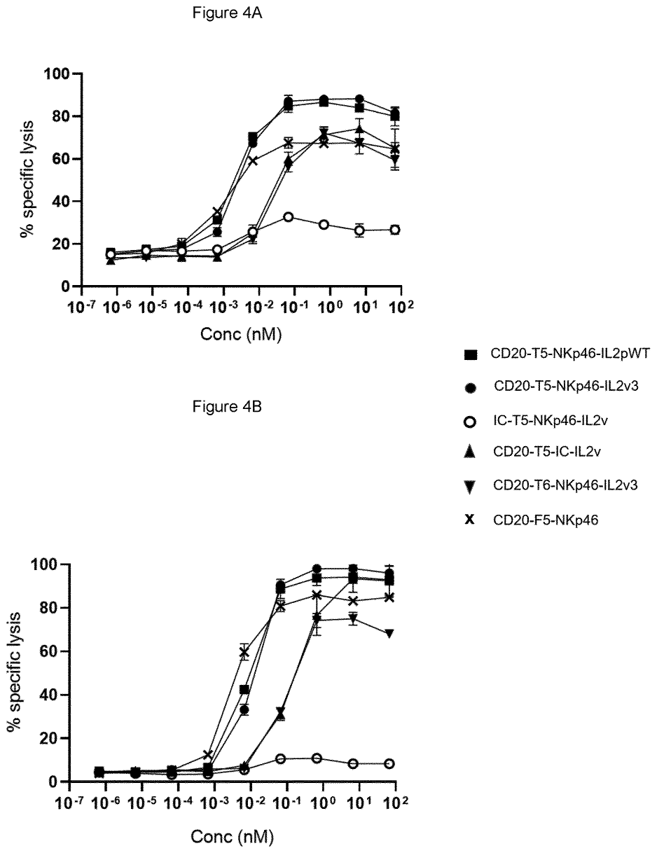

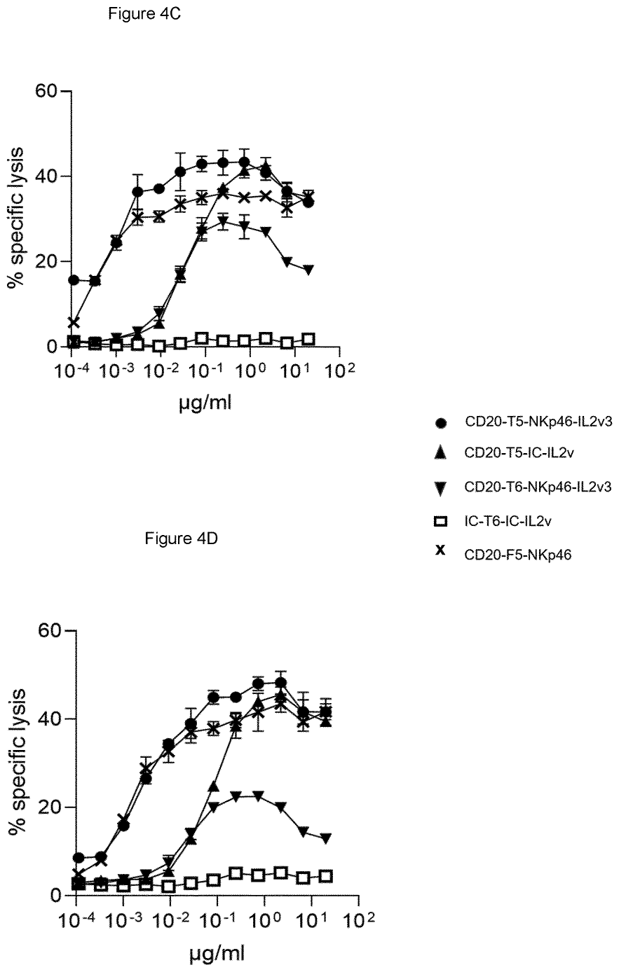

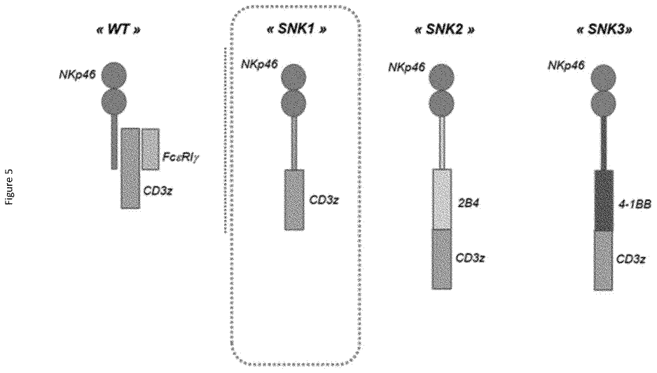

Results are shown in FIGS. 4A and 4B, each panel representing one human NK cell donor, showing % specific lysis induced by NK cells on the y-axis and concentration of test protein on the x-axis. The overall results were consistent across the two human donors. The IC-T5-NKp46-IL2v that lacked binding to CD20 on targeted cells did not induce significant cytotoxicity. All of the CAR potentiator module that retained the ability to bind both CD16 and NKp46 (in addition to CD20) displayed similarly high potency in terms of EC50 values in induction of NK cell cytotoxicity toward the tumor cells. In contrast, the CAR potentiator module that lacked either CD16 or NKp46 binding displayed lower potency. The nature of the IL-2 polypeptide (either wild-type of the mutated IL2v) did not appear to differentially affect NK cell cytotoxicity, and furthermore the presence of IL2, whether as wild-type or IL2v, did not result in improved EC50 values in induction of cytotoxicity compared to the CD20-F5-NKp46 NK cell engager that did not have any IL-2 moiety. However the presence of the IL-2 moiety increased the maximum level of lysis.

### Example 3: CAR Potentiator Module Promotes Tumor Cell Killing in a Standard Vitro Cytotoxicity Assay

In this experiment, CAR potentiator module were assessed for their ability to induce killing of RAJI tumor cells by NK cells from two human donors at effector:target ratio of 2:1 in a standard 4-hour cytotoxicity assay suing Cr⁵¹ as readout.

The test proteins included in this experiment:
CD20-T5-NKp46-IL2v3: contains from topological N- to C-terminus, anti-CD20 VH/VL pair, Fc domain dimer that binds CD16, anti-NKp46-1 VH/VL pair, IL-2v3.
CD20-F5-NKp46: contains from topological N- to C-terminus, anti-CD20 VH/VL pair, Fc domain dimer that binds CD16, anti-NKp46-1 VH/VL pair.
CD20-T5-IC-IL2v: contains from topological N- to C-terminus, anti-CD20 VH/VL pair, Fc domain dimer that binds CD16, IC VH/VL pair, IL2v.
CD20-T6-NKp46-IL2v: contains from topological N- to C-terminus, anti-CD20 VH/VL pair, Fc domain dimer mutated to abolish CD16 binding, anti-NKp46-1 VH/VL pair.
IC-T6-IC-IL2v: contains from topological N- to C-terminus, IC VH/VL pair, Fc domain dimer mutated to abolish CD16 binding, IC VH/VL pair, IL2v.

Briefly, purified NK cells were rested overnight in complete medium. Resting NK cells were then cocultured with Raji tumor cells previously loaded with ⁵¹Cr, in a 2 to 1 ratio. Cells were incubated with test proteins described above (doses from 20 to 0.0001 ug/ml) for 4 h at 37° C., 5.5% CO² in incubator.

Results are shown in FIGS. 4C and 4D each representing one human NK cell donor, showing % specific lysis induced by NK cells on the y-axis and concentration of test protein on the x-axis. The overall results were consistent across the two human donors. The IC-T6-NKp46-IL2v that lacked binding to CD20, CD16 and NKp46 did not induce significant cytotoxicity. All of the CAR potentiator module that retained the ability to bind both CD16 and NKp46 (in addition to CD20) displayed strong ability to potentiate NK cell cytotoxicity toward the tumor cells. The NK cell engagers that lacked either CD16 or NKp46 binding displayed lower potency in terms of EC50 values. The CD20-F5-NKp46 CAR potentiator module which did not have any IL2v moiety was of comparable potency as the CD20-T5-NKp46-IL2v3 CAR potentiator module, although the latter showed a higher plateau of lysis.

### Example 4: NK Cells Engineered to Express CAR Signaling Modules in Combination with CAR Potentiator Modules in Tumor Cell Killing

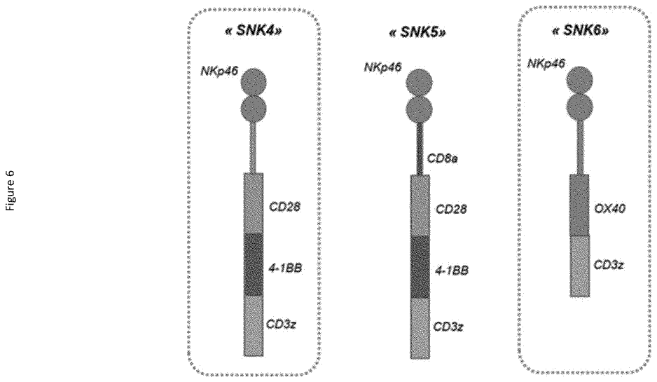

In this experiment, wild-type NK cells (WT) and NK cells engineered to express different CAR signaling modules were tested in cell cytotoxicity assays, in combination with either a CAR potentiator module that binds to the tumor antigen CD20 or a comparator protein lacking the tumor-binding moiety. The engineered NK cells tested were SNK1 (NK cells engineered to express the NKp46-CD3zeta construct), SNK4 (NK cells engineered to express the NKp46-CD28-4-1BB-CD3zeta construct), and SNK6 (NK cells engineered to express the NKp46-OX40-CD3zeta construct). See FIG. 5 and FIG. 6 or the structure of the different CAR signaling module constructs.

NK cells were assessed for their ability to induce killing of Daudi tumor cells by NK cells from human donors in a standard 4-hour cytotoxicity assay suing Cr⁵¹ as readout. Briefly, KHYG-1 cells (WT or genetically engineered) were plated with Daudi tumor cells loaded with Chromium 51 (⁵¹Cr) (PerkinElmer) at several E:T cell ratio (20:1, 10:1 and 5:1) in U-bottomed 96-well plates. The cells were incubated in presence of tested samples for 4 h at 37° C. in RPMI 1640 medium (Gibco) supplemented with 10% heat-inactivated fetal bovine serum (FBS, Gibco), 2 mM L-glutamine (Gibco), 1% non-essential amino acids (Gibco) and 1 mM sodium pyruvate (Gibco) under an atmosphere containing 5% CO². After incubation, 50 μl of the culture supernatant was transferred to a LumaPlate (Perkin Elmer) coated with solid scintillator, which was then placed in a microplate scintillation counter (MicroBeta, Perkin Elmer) to measure 51Cr release into the supernatant, which was correlated with target cell lysis. The following formula was used to calculate the percent specific lysis:

Specific lysis (%)=(experimental release−spontaneous release)/(maximal release−spontaneous release)×100

Maximal ⁵¹Cr release was determined by adding 2% Tergitol to the target cells, and spontaneous release was measured in medium alone, without effector cells.

The CAR potentiator module test proteins included in this experiment were:
CD20-F25-NKp46 (also referred to as CD20-CAR potentiator): contains from topological N- to C-terminus, anti-CD20 VH/VL pair from antibody GA101, Fc domain dimer that binds CD16, anti-NKp46 VH/VL pair. The amino acid sequences of the three chains CD20-F25-NKp46 are shown in SEQ ID NOS: 234, 235 and 236.
IC-F25-NKp46 (also referred to as IC-Potentiator): contains from topological N- to C-terminus, IC VH/VL pair, Fc domain dimer that binds CD16, anti-NKp46 VH/VL pair. The amino acid sequences of the three chains IC-F25-NKp46 are shown in SEQ ID NOS: 234, 235 and 236.

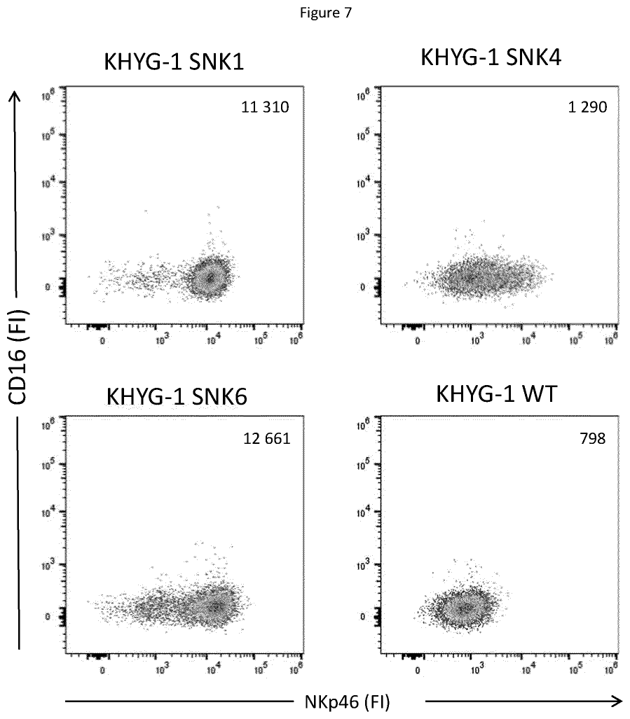

Results of CAR signaling module expression are shown in FIGS. 6 and 7. HEK-F cells were transfected with expression vector encoding SNK1, 2, 3, 4, 5 and 6 NKp46-CAR signaling modules and the expression of the NKp46-CAR at the cell surface was analyzed by flow cytometry. FIG. 7 shows expression of NKp46-CAR signaling modules at the surface of KHYG-1 cells. KHYG-1 cells were transfected with expression vector encoding SNK1, 4, and 6 NKp46-CAR signaling modules. Cells expressing the construct were sorted and the expression of the NKp46-CAR at the cell surface was analyzed by flow cytometry. KHYG-1 WT are the parental cells not transfected. Median fluorescence intensity values of NKp46 expression are indicated in the dot plots.

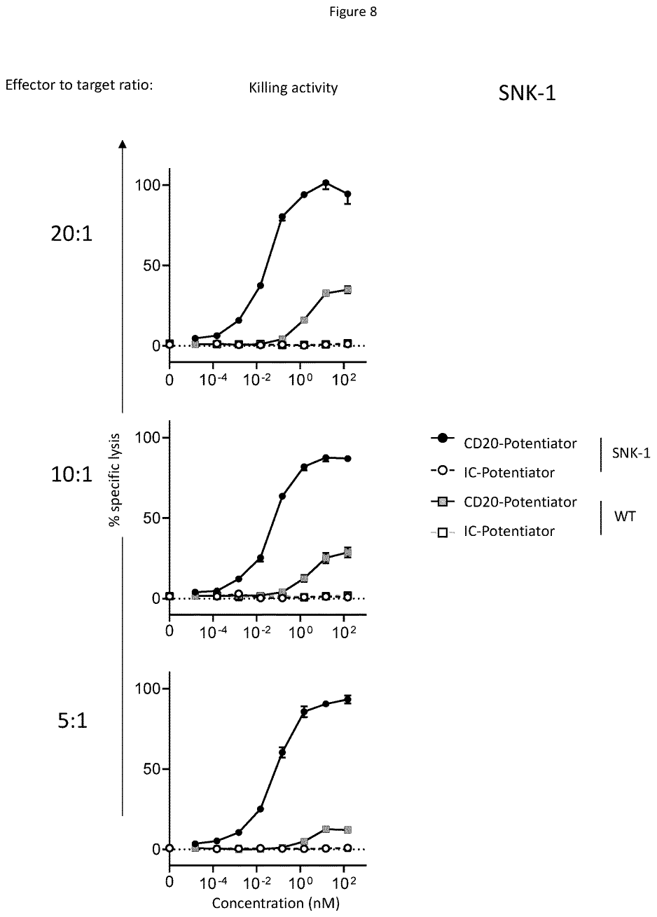

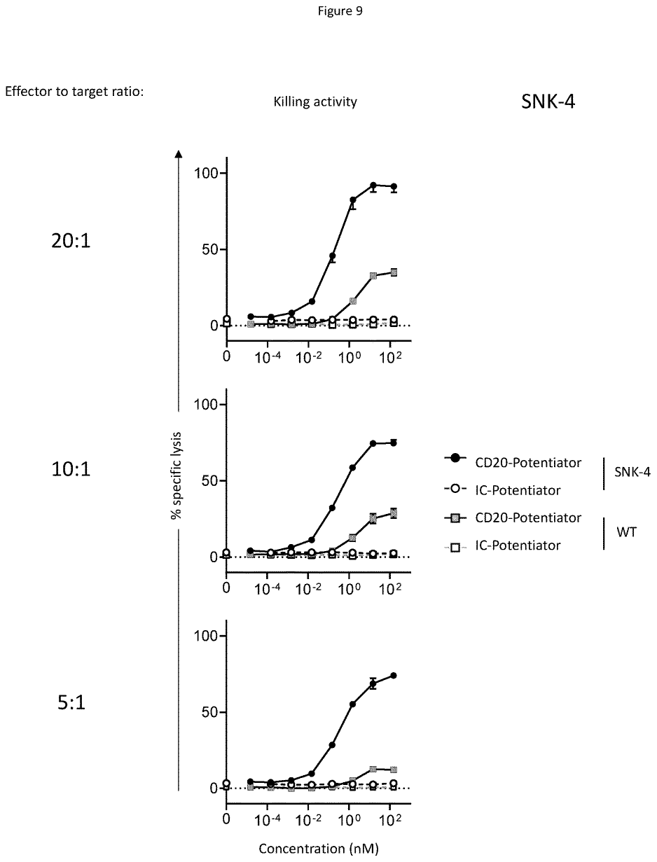

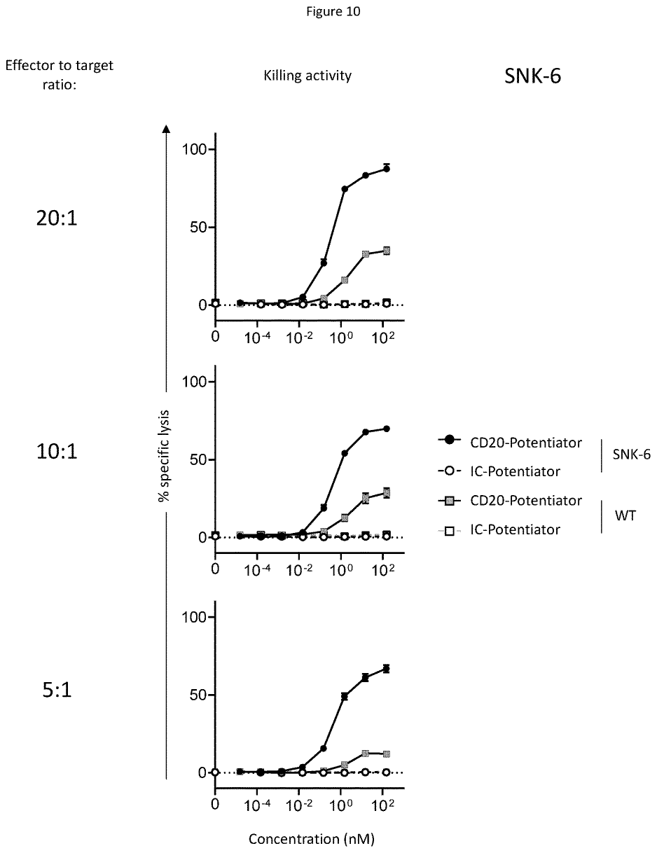

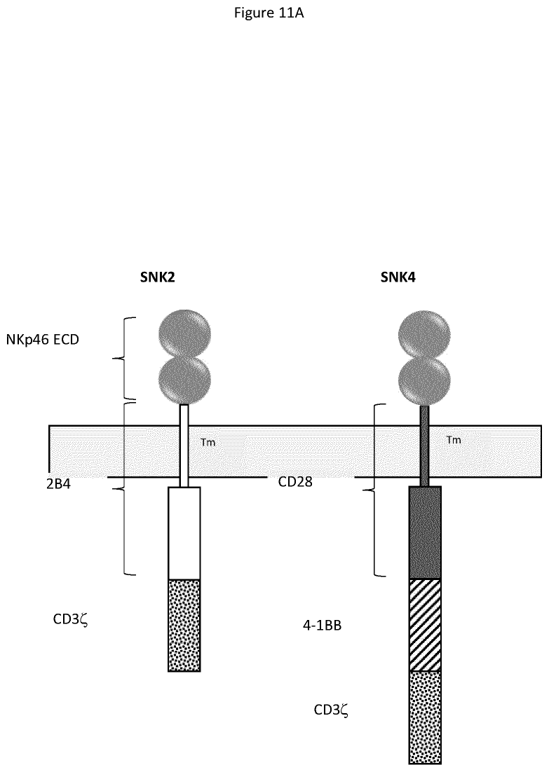

Results of cytotoxicity assays are shown in FIGS. 8-10. FIG. 8 shows cytotoxic activity of KHYG-1 cells expressing NKp46-CAR signaling module SNK1. Cytotoxicity of CD20-CAR potentiator module (CD20-Potentiator) is shown against B cell line Daudi target cells with (SNK1) and without (WT) expression of NKp46-CAR signaling module SNK1 on KHYG-1 cells, used as effectors at effector to target ratios of 20:1, 10:1 and 5:1. FIG. 9 shows cytotoxic activity of KHYG-1 cells expressing NKp46-CAR signaling module SNK4. Cytotoxicity of CD20-CAR potentiator module (CD20-Potentiator) is shown against B cell line Daudi target cells with (SNK4) and without (WT) expression of NKp46-CAR signaling module SNK4 on KHYG-1 cells, used as effectors. FIG. 10 shows cytotoxic activity of KHYG-1 cells expressing NKp46-CAR signaling module SNK6. Cytotoxicity of CD20-CAR potentiator module (CD20-Potentiator) is shown against B cell line Daudi target cells with (SNK6) and without (WT) expression of NKp46-CAR signaling module SNK6 on KHYG-1 cells, used as effectors.

It can thus be observed that the engineered NK cells made to express the CAR signaling modules enhanced the cytotoxic (tumor cell killing) potency of the CAR potentiator module by several orders of magnitude at each E:T ratio tested. Additionally, the combined use of the engineered NK cells with the CAR potentiator module caused a strong increase in the plateau (maximal) target cell lysis that could be achieved compared to wild type NK cells. In the presence of the IC-F25-NKp46 protein that did not bind to CD20, all NK cells, whether WT or engineered, exhibited little cytotoxicity toward the tumor target cells.

### Example 5: CAR-NKp46 Expression at the Cell Surface of Engineered CAR T Cells

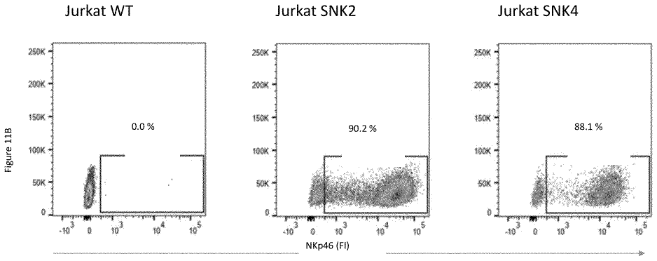

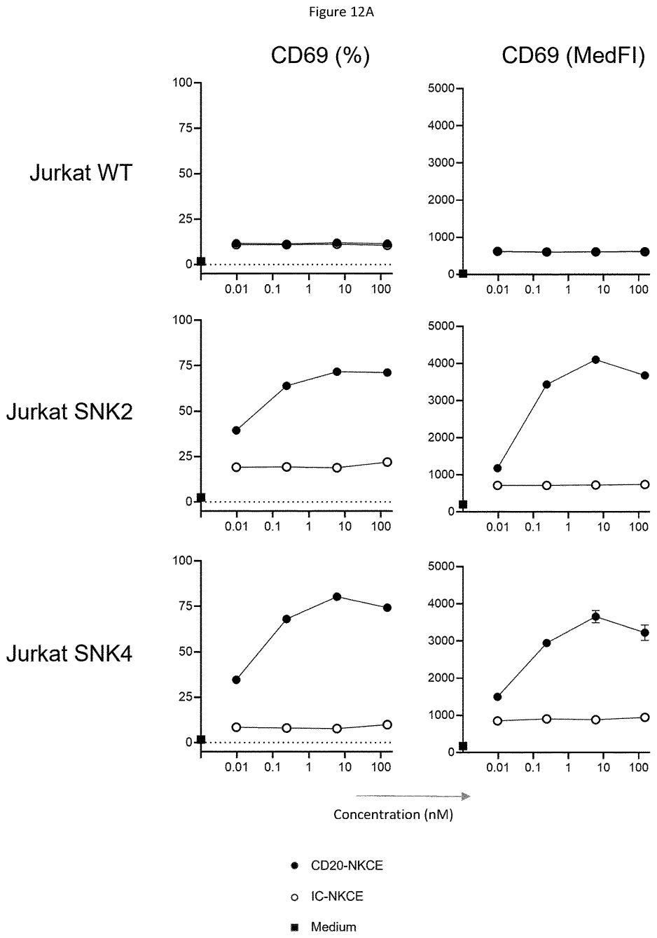

In this experiment, Jurkat T cells engineered to express different CAR signaling modules were evaluated for the expression of the CAR signaling modules. FIG. 11A shows a schematic representation of Jurkat-SNK2 and Jurkat-SNK4 CAR-NKp46 constructs.

FIG. 11B shows results of flow cytometry analysis of the transfected and control Jurkat cells after staining with anti-NKp46 antibody (9E2-PE). Fluorescence intensity (FI) of NKp46 staining is shown on the x-axis and side scatter (SSC) is shown on the y-axis. Percent of NKp46-positive cells are indicated in the graph. The figure shows non-transfected wild-type (WT) Jurkat cells do not express NKp46 ECD at their cell surface, while 90.2% of Jurkat SNK2 cells and 88.1% of Jurkat SNK4 cells expressed NKp46 ECD at their cell surface.

### Example 6: NKCE Mediates Target-Specific Activation of T Cells Expressing CAR-NKp46 Constructs

In this experiment, wild-type Jurkat T cells (WT) and Jurkat T cells engineered to express SNK2 or SNK4 CAR signaling modules were tested in T cell activation assays, in combination with either a CAR potentiator module that binds to the tumor antigen CD20 (CD20-NKCE) or a comparator protein lacking the tumor-binding moiety (IC-NKCE). The engineered T cells tested were Jurkat-SNK2 and Jurkat-SNK4 having the structures shown in FIG. 11A.

The Jurkat T cells (WT, SNK2 or SNK4) and target cells (Daudi cells) were incubated for 24 h at 37° C. in presence of CD20 targeting CAR potentiator module (CD20-NKCE) or isotype control (IC-NKCE) molecules at increasing concentrations of 0.01, 0.25, 6 and 150 nM. The expression of CD25 and CD69 activation markers was analyzed after gating on CD3-positive alive cells (i.e. Jurkat alive cells). The CAR potentiator module and isotype control used were CD20-F25-NKp46 and IC-F25-NKp46 as in Example 4.

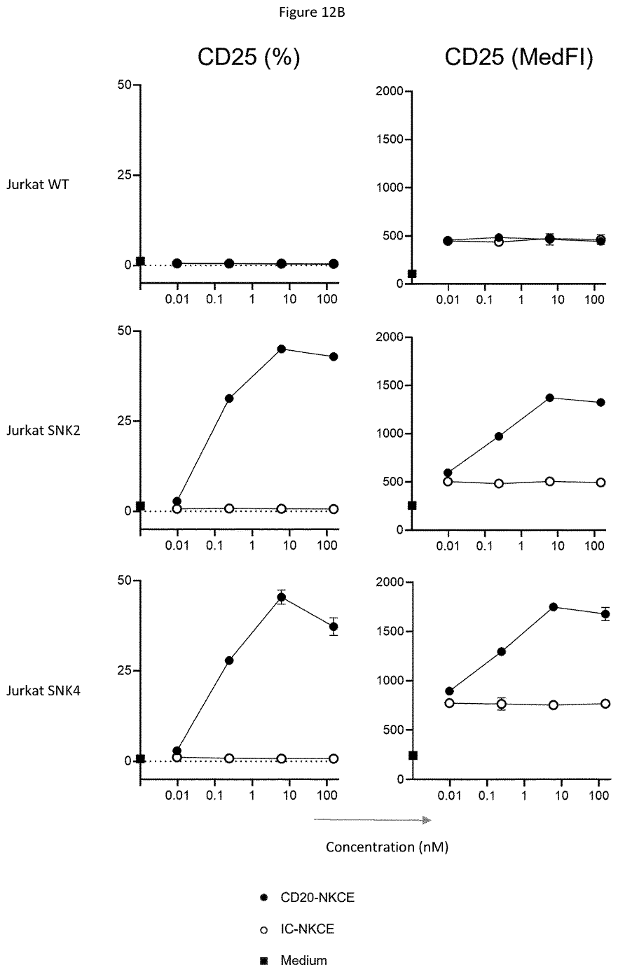

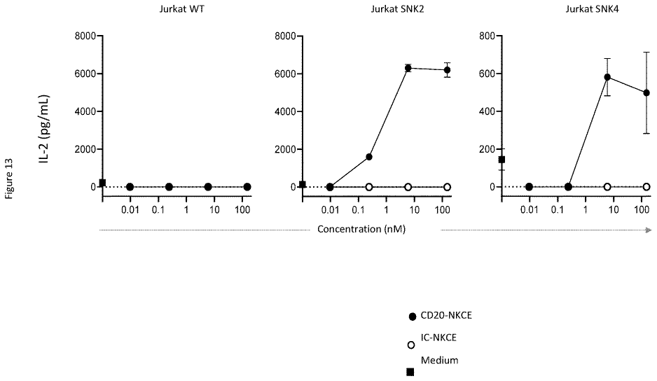

The results showed that the CD20-NKCE CAR potentiator module induces target dependent CD69 & CD25 expression at the cell surface of Jurkat SNK2 & SNK4, in a dose dependent manner. Results are shown in FIGS. 12A and 12B for CD69 and CD25 expression, respectively. The x-axis shows concentration of test molecules and the y-axis shows median fluorescence intensity (MedFI) on the right hand panels and on the left hand panels percent positive cells expressing CD69 and CD25 at the cell surface for parental Jurkat (WT) and Jurkat cells expressing SNK2 and SNK4 constructs.

The CD20-NKCE CAR potentiator module was further evaluated for to assess its ability to induces target antigen dependent II-2 secretion by the wild-type Jurkat T cells (WT) and Jurkat T cells engineered to express SNK2 or SNK4 CAR signaling modules.

The Jurkat T cells (WT, SNK2 or SNK4) and target cells (Daudi cells) were incubated for 24 h in presence of CD20 targeting (CD20-NKCE) and isotype control (IC-NKCE) molecules at increasing concentrations of 0.01, 0.25, 6 and 150 nM. The secretion of II-2 in culture supernatant was monitored by ELISA (ULOQ 500 μg/mL).

Results showed that the CD20-NKCE CAR potentiator module induces target dependent IL-2 secretion by Jurkat SNK2 & SNK4 cells, in a dose dependent manner. Results are shown in FIG. 13. The x-axis shows concentration of test molecules and the y-axis shows IL-2 concentrations (pg/mL). The SNK2 construct appeared more efficient than SNK4 to promote IL-2 secretion by Jurkat T cells.

### Example 7: NK Cell Line Sub-Clones Expressing CD3z, or FceRIg

The parental KHYG-1 cell line used as NK cells showed low endogenous expression of CD16a and NKp46 at the cell surface, both of which normally associate with the accessory molecule CD3z and FceRIg to form functional receptors. We hypothesized that the altered expression of CD16a and NKp46 might be due to a default in the expression or function of CD3z and/or FceRIg in KHYG-1 cells.

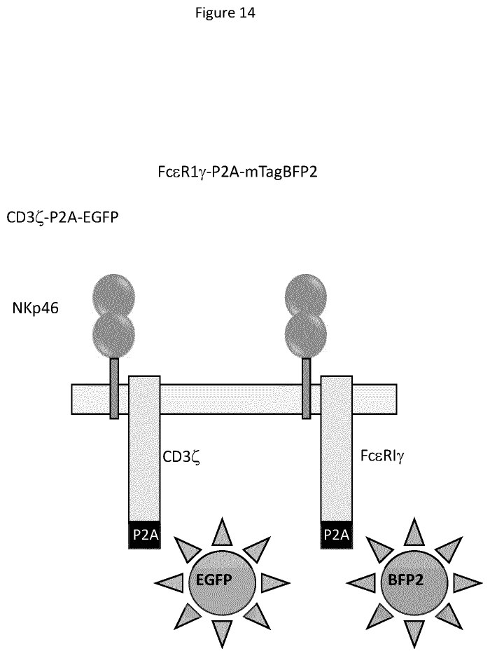

NK cells (KHYG-1 cell line) were modified by transfection to express CD3z or FceRIg in order to complement the existing NKp46 expression in the NK cells. FIG. 14 shows a schematic representation of CD3z and FceRIg constructs used to complement regular NKp46 cell surface expression in KHYG-1 cells.

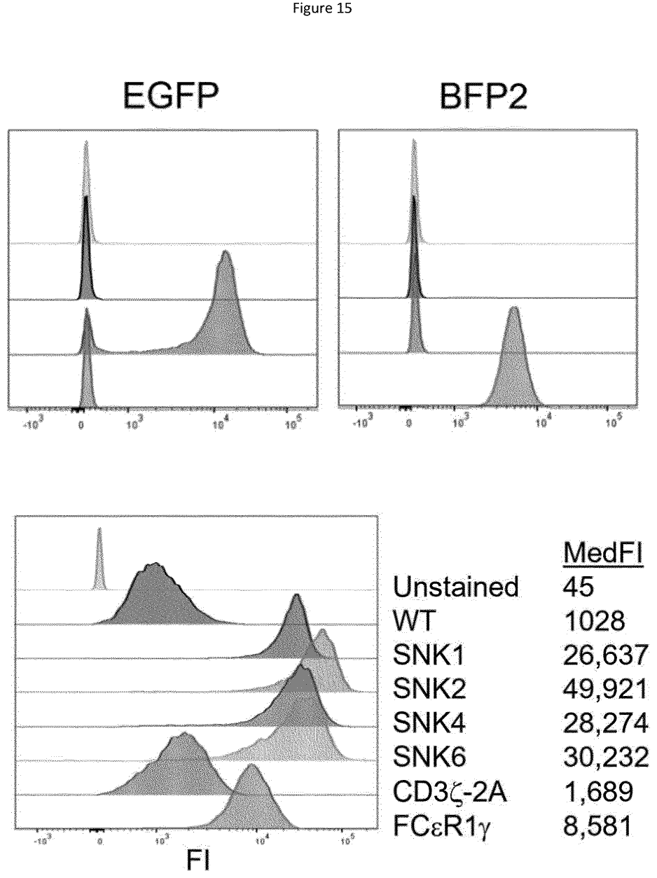

FIG. 15, top panel, shows expression of EGFP and BFP2 associated to CD3z and FceRIg constructs in KHYG-1 sub-clones monitored by flow cytometry. FIG. 15, bottom panel, shows expression of NKp46 at the cell surface of KHYG-1 sub-clones expressing CAR-NKP46, CD3z and FceRIg constructs. Values of median fluorescence intensity (MedFI) are indicated on the graphs.

We observed that complementation of KHYG-1 cells with accessory molecules promoted a higher expression of NKp46 at the cell surface and that the expression promoted by FceRIg was superior to the expression observed with CD3z.

### Example 8: Cytotoxicity of Parental NK Cells and NK Cell Sub-Clones Expressing CAR-NKp46, CD3z, or FceRIg Constructs

In this experiment, we compared the cytotoxicity against Daudi tumor cells mediated by parental KHYG-1 NK cells and KHYG-1 sub-clones expressing CAR-NKp46, CD3z, or FceRIg constructs, in the presence or absence of a CAR potentiator module that binds to the tumor antigen CD20 (CD20-NKCE) or a comparator protein lacking the tumor-binding moiety (IC-NKCE). CAR potentiator modules were as described in Example 4.

Daudi cells were loaded with 51 chromium and incubated with effector KHYG-1 cells at an effector to target (E/T) ratio of 5:1 or 1:1 in presence of increasing concentrations (from 0.01 to 150 nM) of CD20-NKCE CAR potentiator module (CD20-NKCE) or isotype control NKCE (IC-NKCE). The % specific lysis of Daudi targets are shown for parental KHYG-1 (WT), KHYG-1 sub-clones expressing CAR-NKp46 constructs (SNK1, SNK2, SNK4, and SNK6), or CD3z and FceRIg constructs. The CAR potentiator module and isotype control used were CD20-F25-NKp46 and IC-F25-NKp46 as in Example 4.

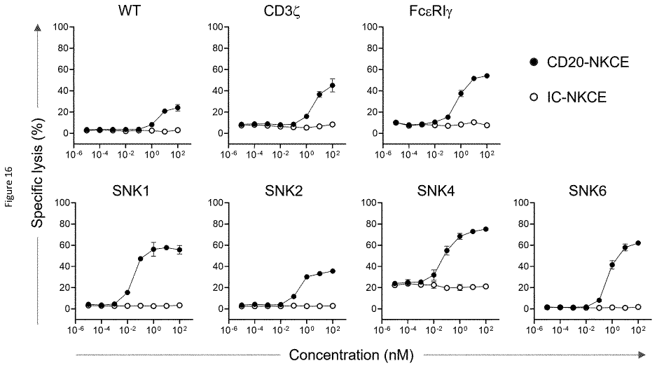

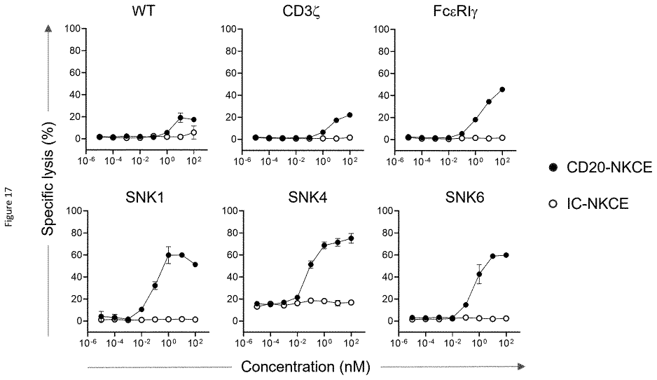

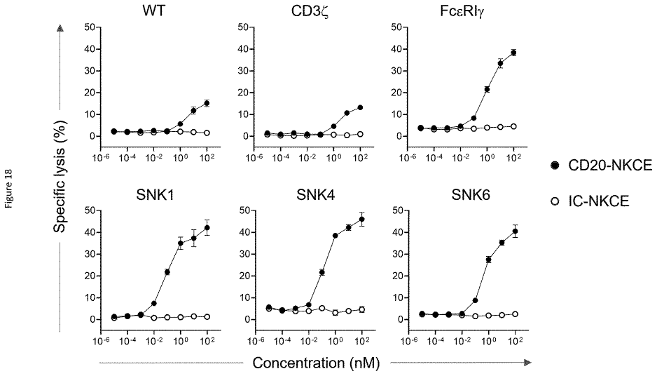

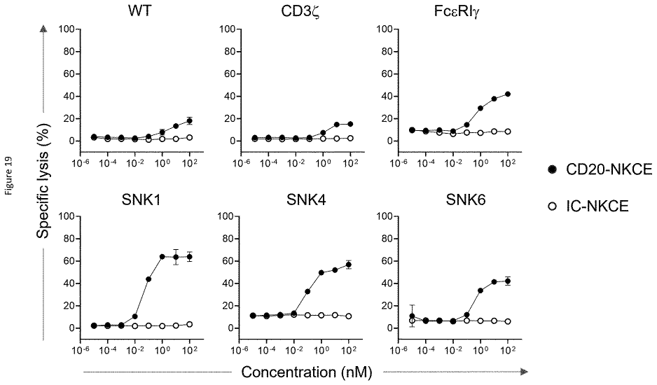

FIGS. 16, 17, 18 and 19 show the results from four different experiments, including two experiments conducted with NK cells at an E/T ratio of 5:1 and two experiments conducted with NK cells at an E/T ratio of 1:1. FIG. 16 and FIG. 17 respectively show a first and a second experiment with NK cells at an E/T ratio of 5:1. FIG. 18 and FIG. 19 respectively show a first and a second experiment with NK cells at an E/T ratio of 1:1. The x-axis shows concentration (nM) of the CAR potentiator module and the y-axis shows % specific lysis of Daudi target cells.

Results showed that expression of FcerIg in KHYG-1 improved the ANKET-mediated killing efficacy and potency as compared to parental KHYG-1, contrary to CD3z for which the gain of ANKET-mediated activity was very moderate. This is consistent with the observation that complementation of KHYG-1 cells with accessory molecules promoted a higher expression of NKp46 at the cell surface and that FceRIg was superior to CD3z.

KHYG-1 clone expressing SNK6 construct showed similar ANKET-killing activity as the FceRIg clone, and those expressing SNK1 and SNK4 were superior. Results showed that in each case, the CAR potentiator module (CD20-NKCE) permitted a specific targeting of the tumor cells by the NK cells. In the presence of the comparator protein lacking the tumor-binding moiety (IC-NKCE), the NK cells did not mediate lysis of the Daudi tumor cells. The CAR potentiator module permitted parental KHYG-1 NK cells and KHYG-1 sub-clones expressing CAR-NKp46, CD3z, or FceRIg constructs to lyse the tumor cells in a dose-dependent manner. The tumor cell lysis was most potent with the CAR-NKp46-expressing NK cells (SNK1, SNK2, SNK4 and SNK6), and particularly at the lower E/T ratio of 1:1.

### Example 9: Epitope Mapping of Anti-NKp46 Antibodies

### A. Competition Assays

Competition assays were conducted by Surface Plasmon Resonance (SPR according to the methods described below).

SPR measurements were performed on a Biacore T100 apparatus (Biacore GE Healthcare) at 25° C. In all Biacore experiments HBS-EP+ (Biacore GE Healthcare) and NaOH 10 mM NaCl 500 mM served as running buffer and regeneration buffer respectively. Sensorgrams were analyzed with Biacore T100 Evaluation software. Anti-6×His tag antibody was purchased from QIAGEN. Human 6×His tagged NKp46 recombinant proteins (NKp46-His) were cloned, produced and purified at Innate Pharma.

Anti-His antibodies were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Protein-A and anti-His antibodies were diluted to 10 μg/ml in coupling buffer (10 mM acetate, pH 5.6) and injected until the appropriate immobilization level was reached (i.e. 2000 to 2500 RU). Deactivation of the remaining activated groups was performed using 100 mM ethanolamine pH 8 (Biacore GE Healthcare).

Parental regular human IgG1 chimeric antibodies having NKp46 binding region corresponding to NKp46-1, NKp46-2, NKp46-3 or NKp46-4 were used for the competition study which has been performed using an Anti-6×His tag antibody chip. Bispecific antibodies having NKp46 binding region based on NKp46-1, NKp46-2, NKp46-3 or NKp46-4 at 1 μg/mL were captured onto Protein-A chip and recombinant human NKp46 proteins were injected at 5 μg/mL together with a second test bispecific antibody of the NKp46-1, NKp46-2, NKp46-3 or NKp46-4 group. The results demonstrated that none of NKp46-1, NKp46-2, NKp46-3 or NKp46-4 competed with one another for binding to NKp46. Accordingly these antibodies each bind or interact with a different NKp46 epitope.

### B. Binding to NKp46 Mutants

In order to define the epitopes of these anti-NKp46 antibodies, NKp46 mutants were designed, each defined by one, two or three substitutions of amino acids exposed at the molecular surface over the 2 domains of NKp46. This approach led to the generation of 42 mutants which were transfected in Hek-293T cells, as shown in the table below. The targeted amino acid mutations in Table G below are shown both according to the numbering of SEQ ID NO: 1 (also corresponding to the numbering used in Jaron-Mendelson et al. (2012) J. Immunol. 88(12):6165-74.

TABLE G

| | Substitution | | |
|---|---|---|---|
| | (Numbering according to Jaron- | | |
| Mutant | Mendelson and SEQ ID NO: 1) | | |
| 1 | P40A | K43S | Q44A |
| 2 | K41S | E42A | E119A |
| 3 | P86A | D87A | |
| 4 | N89A | R91A | |
| 5 | K80A | K82A | |
| 5bis | E34A | T46A | |
| 6 | R101A | V102A | |
| 7 | N52A | Y53A | |
| 8 | V56A | P75A | E76A |
| 9 | R77A | I78A | |
| 10 | S97A | I99A | |
| 10bis | Q59A | H61A | |
| 11 | L66A | V69A | |
| 12 | E108A | | |
| 13 | N111A | L112A | |
| 14 | D114A | | |
| 15 | T125A | R145S | D147A |
| 16 | S127A | Y143A | |
| 17 | H129A | K139A | |
| 18 | K170A | V172A | |
| 19 | I135A | S136A | |
| 19bis | T182A | R185A | |
| 20 | R160A | | |
| 21 | K207A | | |
| 22 | M152A | R166A | |
| 23 | N195A | N196A | |
| Stalk1 | D213A | I214A | T217A |
| Stalk2 | F226A | T233A | |
| Stalk3 | L236A | T240A | |
| Supp1 | F30A | W32A | |
| Supp2 | F62A | F67A | |
| Supp3 | E63A | Q95A | |
| Supp4 | R71A | K73A | |
| Supp5 | Y84A | | |
| Supp6 | E104A | L105A | |
| Supp7 | Y121A | Y194A | |
| Supp8 | P132A | E133A | |
| Supp9 | S151A | Y168A | |
| Supp10 | S162A | H163A | |
| Supp11 | E174A | P176A | |
| Supp12 | P179A | H184A | |
| Supp13 | R189A | E204A | P205A |

### C. Generation of Mutants

NKp46 mutants were generated by PCR. The sequences amplified were run on agarose gel and purified using the Macherey Nagel PCR Clean-Up Gel Extraction kit. Two or three purified PCR products generated for each mutant were then ligated into an expression vector, with the ClonTech InFusion system. The vectors containing the mutated sequences were prepared as Miniprep and sequenced. After sequencing, the vectors containing the mutated sequences were prepared as Midiprep using the Promega PureYield™ Plasmid Midiprep System. HEK293T cells were grown in DMEM medium (Invitrogen), transfected with vectors using Invitrogen's Lipofectamine 2000 and incubated at 37° C. in a CO2 incubator for 24 hours prior to testing for transgene expression.

### D. Flow Cytometry Analysis of Anti-NKp46 Binding to the HEK293T Transfected Cells

All the anti-NKp46 antibodies were tested for their binding to each mutant by flow cytometry. A first experiment was performed to identify antibodies that lose their binding to one or several mutants at a particular concentration (10 μg/ml). To confirm the loss of binding, titration of antibodies was done using antibodies for which binding seemed to be affected by the NKp46 mutations (1-0.1-0.01-0.001 μg/ml).

### E. Results

Antibody NKp46-1 had decreased binding to the mutant 2 (having a mutation at residues K41, E42 and E119) (numbering in NKp46 wild-type) compared to wild-type NK46. Similarly, NKp46-1 also had decreased binding to the supplementary mutant Supp7 (having a mutation at residues Y121 and Y194).

Antibody NKp46-3 had decreased binding to the mutant 19 (having a mutation at residues I135, and S136). Similarly, NKp46-3 also had decreased binding to the supplementary mutant Supp8 (having a mutation at residues P132 and E133).

Antibody NKp46-4 had decreased binding to the mutant 6 (having a mutation at residues R101, and V102). Similarly, NKp46-4 also had decreased binding to the supplementary mutant Supp6 having a mutation at residues E104 and L105.

Epitopes of NKp46-4, NKp46-3 and NKp46-1 were found to be on the NKp46 D1 domain, D2 domain and D1/D2 junction, respectively. R101, V102, E104 and L105 are essential residues for NKp46-4 binding and defined a part of NKp46-4 epitope. The epitope of NKp46-1 epitope includes K41, E42, E119, Y121 and Y194 residues. The epitope of NKp46-3 includes P132, E133, I135, and S136 residues.

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e. g., all exact exemplary values provided with respect to a particular factor or measurement can be considered to also provide a corresponding approximate measurement, modified by “about,” where appropriate). All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope unless otherwise indicated. No language in the specification should be construed as indicating any element is essential to the practice unless as much is explicitly stated.

The description herein of any aspect or embodiment using terms such as reference to an element or elements is intended to provide support for a similar aspect or embodiment that “consists of”, “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A chimeric activating receptor (CAR) signaling module comprising: (i) an extracellular domain of a NKp46 protein or fragment thereof; (ii) a transmembrane domain; and (iii) an intracellular signaling domain.

2. The CAR signaling module of claim 1, wherein said extracellular domain of a NKp46 protein comprises the amino acid sequence of SEQ ID NO: 4, a sequence at least 80%, 90% or 95% identical thereto, or a functional fragment thereof.

3. The CAR signaling module of claim 1 or 2, wherein said extracellular domain of a NKp46 protein or fragment thereof binds to an anti-NKp46 antibody.

4. The CAR signaling module of any one of claims 1, 2 or 3, wherein said extracellular domain of a NKp46 protein or fragment thereof is capable of binding to an antibody comprising a heavy chain variable region (VH) of SEQ ID NOS: 146 or 147, and a light chain variable region of SEQ ID NO: 148.

5. The CAR signaling module of any one of claims 1-4, wherein said transmembrane domain is a transmembrane domain from a protein selected from the group consisting of CD3 zeta, 2B4, DAP10, DAP12, 4-1BB, CD28, CD8 alpha, OX40.

6. The CAR signaling module of any one of claims 1-5, wherein said intracellular signaling domain comprises a functional signaling domain of CD3 zeta.

7. The CAR signaling module of claim 6, wherein said intracellular signaling domain further comprises at least one costimulatory signaling domain comprising a functional domain of a protein selected from the group consisting of 2B4, DAP10, DAP12, 4-1BB, CD28, OX40.

8. The CAR signaling module of claims 1-6, wherein said intracellular signaling domain comprises a functional signaling domain of CD3 zeta comprising an amino acid sequence of SEQ ID NO: 18.

9. The CAR signaling module of claims 1-7, wherein said intracellular signaling domain comprises a costimulatory signaling domain of 2B4 comprising an amino acid sequence of SEQ ID NO: 19.

10. The CAR signaling module of claims 1-7, wherein said intracellular signaling domain comprises a functional signaling domain of DAP10 comprising an amino acid sequence of SEQ ID NO: 20

11. The CAR signaling module of claims 1-7, wherein said intracellular signaling domain comprises a functional signaling domain of DAP12 comprising an amino acid sequence of SEQ ID NO: 21.

12. The CAR signaling module of claims 1-7, wherein said intracellular signaling domain comprises a functional signaling domain of 4-1BB comprising an amino acid sequence of SEQ ID NO: 22.

13. The CAR signaling module of claims 1-7, wherein said intracellular signaling domain comprises a functional signaling domain of CD28 comprising an amino acid sequence of SEQ ID NO: 23.

14. The CAR signaling module of claims 1-7, wherein said intracellular signaling domain comprises a functional signaling domain of OX40 comprising an amino acid sequence of SEQ ID NO: 24.

15. The CAR signaling module of claims 1-6, wherein said CAR signaling module comprises a transmembrane domain of CD3 zeta, an intracellular signaling domain of CD3 zeta, optionally wherein the CAR signaling module comprises the amino acid sequence of SEQ ID NOS: 29 or 30.

16. The CAR signaling module of claims 1-7, wherein said CAR signaling module comprises a transmembrane domain of 2B4, a costimulatory signaling domain of 2B4, a intracellular signaling domain of CD3 zeta, optionally wherein the CAR signaling module comprises the amino acid sequence of SEQ ID NOS 31 or 32.

17. The CAR signaling module of claims 1-7, wherein said CAR signaling module comprises a transmembrane domain of DAP10, a costimulatory signaling domain of DAP10, an intracellular signaling domain of CD3 zeta, optionally wherein the CAR signaling module comprises the amino acid sequence of SEQ ID NOS: 33 or 34.

18. The CAR signaling module of claims 1-7, wherein said CAR signaling module comprises a transmembrane domain of DAP12, a costimulatory signaling domain of DAP12, an intracellular signaling domain of CD3 zeta, optionally wherein the CAR signaling module comprises the amino acid sequence of SEQ ID NOS: 35 or 36.

19. The CAR signaling module of claims 1-7, wherein said CAR signaling module comprises a transmembrane domain of CD28, a costimulatory signaling domain of CD28, an intracellular signaling domain of CD3 zeta, optionally wherein the CAR signaling module comprises the amino acid sequence of SEQ ID NOS 37 or 38.

20. The CAR signaling module of claims 1-7, wherein said CAR signaling module comprises a transmembrane domain of CD28, a first costimulatory signaling domain of CD28, a second costimulatory signaling domain of 4-1BB, an intracellular signaling domain of CD3 zeta, optionally wherein the CAR signaling module comprises the amino acid sequence of SEQ ID NOS: 39 or 40.

21. The CAR signaling module of claims 1-7, wherein said CAR signaling module comprises a transmembrane domain of 4-1BB, a costimulatory signaling domain of 4-1 BB, an intracellular signaling domain of CD3 zeta, optionally wherein the CAR signaling module comprises the amino acid sequence of SEQ ID NO: 41.

22. The CAR signaling module of claims 1-7, wherein said CAR signaling module comprises a transmembrane domain of CD8 alpha, a costimulatory signaling domain of CD28, an intracellular signaling domain of CD3 zeta, optionally wherein the CAR signaling module comprises the amino acid sequence of SEQ ID NO: 42.

23. The CAR signaling module of claims 1-7, wherein said CAR signaling module comprises a transmembrane domain of CD8 alpha, a costimulatory signaling domain of 4-1EE, an intracellular signaling domain of CD3 zeta, optionally wherein the CAR signaling module comprises the amino acid sequence of SEQ ID NO: 43.

24. The CAR signaling module of claims 1-7, wherein said CAR signaling module comprises a transmembrane domain of CD8 alpha, a first costimulatory signaling domain of CD28, a second costimulatory signaling domain of 4-1EE, an intracellular signaling domain of CD3 zeta, optionally wherein the CAR signaling module comprises the amino acid sequence of SEQ ID NO: 44.

25. The CAR signaling module of claims 1-7, wherein said CAR signaling module comprises a transmembrane domain of OX40, a costimulatory signaling domain of OX40, an intracellular signaling domain of CD3 zeta, optionally wherein the CAR signaling module comprises the amino acid sequence of SEQ ID NO: 45.

26. The CAR signaling module of claims 1-7, wherein said CAR signaling module comprises a transmembrane domain of OX40, a first costimulatory signaling domain of OX40, a second costimulatory signaling domain of 4-1EE, an intracellular signaling domain of CD3 zeta, optionally wherein the CAR signaling module comprises the amino acid sequence of SEQ ID NO: 46.

27. The CAR signaling module of claims 1-7, wherein said CAR signaling module comprises a transmembrane domain of CD8 alpha, a costimulatory signaling domain of OX40, an intracellular signaling domain of CD3 zeta, optionally wherein the CAR signaling module comprises the amino acid sequence of SEQ ID NO: 47.

28. The CAR signaling module of claims 1-7, wherein said CAR signaling module comprises a transmembrane domain of CD8 alpha, a first costimulatory domain of OX40, a second costimulatory signaling domain of 4-1BB, an intracellular signaling domain of CD3 zeta, optionally wherein the CAR signaling module comprises the amino acid sequence of SEQ ID NO: 48.

29. A modular CAR system comprising: (A) a signaling module of any one of claim 1-28, and (B) a potentiator module comprising (1) an NKp46 binding moiety and (2) a target cell binding moiety.

30. The modular CAR of claim 29, wherein the potentiator module further comprises an Fc domain, or a portion an Fc domain capable of binding FcRn.

31. The modular CAR of any one of claim 29 or 30, wherein the potentiator module further comprises a binding domain that binds a human cytokine receptor present on NK cells.

32. The modular CAR of claim 31, wherein the binding domain that binds a human cytokine receptor present on NK cells comprises an IL-2 moiety, or a variant thereof.

33. The modular CAR of any one of claims 29-32, wherein said NKp46 binding moiety comprised in the potentiator module is capable of binding the extracellular NKp46 protein or fragment thereof comprised in the signaling module.

34. A nucleic acid or set of nucleic acids encoding a CAR signaling module of any one of claims 1-28.

35. An expression vector comprising a nucleic acid of claim 34.

36. A cell genetically modified to express a CAR signaling module of any one of claims 1-28 or comprising nucleic acids or an expression vector of claims 34-35.

37. The cell of claim 36, wherein the cell is a T cell or a NK cell.

38. The cell of any one of claim 36-37, wherein said cell is allogeneic or autologous.

39. A composition comprising cells as defined in any one of claims 36-38, and a pharmaceutically acceptable carrier.

40. The cell as defined in any one of claims 36-38, for use as a medicament.

41. A kit comprising a first composition comprising a cell as defined in claim 36-39 and a second composition comprising a CAR potentiator module comprising (1) an NKp46 binding moiety and (2) a target cell binding moiety, optionally wherein the kit is for simultaneous, sequential or separate administration to an individual.

42. A method for preparing a cell composition, comprising: providing a cell, optionally wherein the cell is a T cell or an NK cell; and introducing to the cell a nucleic acid or set of nucleic acids encoding a CAR signaling module of any one of claims 1-28, under conditions suitable such that the cell expresses at its surface the CAR signaling module.

43. A method of treating an individual comprising administering to said individual: (A) a cell of any one of claims 36-38, and (B) a CAR potentiator module comprising (1) an NKp46 binding moiety and (2) a target cell binding moiety.

44. A composition comprising cells as defined in any one of claims 36-38, for use in treatment of disease in combination with a CAR potentiator module comprising (1) an NKp46 binding moiety and (2) a target cell binding moiety.

45. A CAR potentiator module comprising (1) an NKp46 binding moiety and (2) a target cell binding moiety, for use in treatment of disease in combination with a composition comprising cells as defined in any one of claims 36-38.

46. The cell, composition, kit, use or method of claims 36-45 for use in the treatment of a cancer.


SEQUENCE LISTING (239 sequences)

SEQ ID NO: 1 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 2 - protein, 258 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLR

SEQ ID NO: 3 - protein, 237 aa, synthetic construct
QQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLR

SEQ ID NO: 4 - protein, 236 aa, synthetic construct
QQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLL

SEQ ID NO: 5 - protein, 21 aa, synthetic construct
LCYLLDGILFIYGVILTALFL

SEQ ID NO: 6 - protein, 24 aa, synthetic construct
DPKLCYLLDGILFIYGVILTALFL

SEQ ID NO: 7 - protein, 21 aa, synthetic construct
FLVIIVILSALFLGTLACFCV

SEQ ID NO: 8 - protein, 24 aa, synthetic construct
FWPFLVIIVILSALFLGTLACFCV

SEQ ID NO: 9 - protein, 21 aa, synthetic construct
LLAGLVAADAVASLLIVGAVF

SEQ ID NO: 10 - protein, 23 aa, synthetic construct
LPLLAGLVAADAVASLLIVGAVF

SEQ ID NO: 11 - protein, 21 aa, synthetic construct
GVLAGIVMGDLVLTVLIALAV

SEQ ID NO: 12 - protein, 23 aa, synthetic construct
SPGVLAGIVMGDLVLTVLIALAV

SEQ ID NO: 13 - protein, 27 aa, synthetic construct
IISFFLALTSTALLFLLFFLTLRFSVV

SEQ ID NO: 14 - protein, 27 aa, synthetic construct
FWVLVVVGGVLACYSLLVTVAFIIFWV

SEQ ID NO: 15 - protein, 28 aa, synthetic construct
PFWVLVVVGGVLACYSLLVTVAFIIFWV

SEQ ID NO: 16 - protein, 21 aa, synthetic construct
IYIWAPLAGTCGVLLLSLVIT

SEQ ID NO: 17 - protein, 21 aa, synthetic construct
VAAILGLGLVLGLLGPLAILL

SEQ ID NO: 18 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 20 - protein, 24 aa, synthetic construct
LCARPRRSPAQEDGKVYINMPGRG

SEQ ID NO: 21 - protein, 52 aa, synthetic construct
YFLGRLVPRGRGAAEAATRKQRITETESPYQELQGQRSDVYSDLNTQRPYYK

SEQ ID NO: 22 - protein, 42 aa, synthetic construct
KRGRKKLLYIFKQPFMRPVQTTQEEDGCSCRFPEEEEGGCEL

SEQ ID NO: 23 - protein, 41 aa, synthetic construct
RSKRSRLLHSDYMNMTPRRPGPTRKHYQPYAPPRDFAAYRS

SEQ ID NO: 24 - protein, 42 aa, synthetic construct
ALYLLRRDQRLPPDAHKPPGGGSFRTPIQEEQADAHSTLAKI

SEQ ID NO: 25 - protein, 5 aa, synthetic construct
GSSSS

SEQ ID NO: 26 - protein, 10 aa, synthetic construct
GSSSSGSSSS

SEQ ID NO: 27 - protein, 12 aa, synthetic construct
GSSSSGSSSSGS

SEQ ID NO: 28 - protein, 15 aa, synthetic construct
GSSSSGSSSSGSSSS

SEQ ID NO: 29 - protein, 392 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - protein, 394 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 31 - protein, 512 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 32 - protein, 515 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 33 - protein, 416 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRLLAGLVAADAVASLLIVGAVFLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 34 - protein, 418 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRLPLLAGLVAADAVASLLIVGAVFLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 35 - protein, 444 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRGVLAGIVMGDLVLTVLIALAVYFLGRLVPRGRGAAEAATRKQRITETESPYQELQGQRSDVYSDLNTQRPYYKRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 36 - protein, 446 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRSPGVLAGIVMGDLVLTVLIALAVYFLGRLVPRGRGAAEAATRKQRITETESPYQELQGQRSDVYSDLNTQRPYYKRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 37 - protein, 439 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 38 - protein, 440 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 39 - protein, 481 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 482 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 41 - protein, 440 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - skipped

SEQ ID NO: 43 - protein, 434 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - protein, 475 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRIYIWAPLAGTCGVLLLSLVITRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 45 - protein, 434 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRVAAILGLGLVLGLLGPLAILLALYLLRRDQRLPPDAHKPPGGGSFRTPIQEEQADAHSTLAKIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 476 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRVAAILGLGLVLGLLGPLAILLALYLLRRDQRLPPDAHKPPGGGSFRTPIQEEQADAHSTLAKIKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 47 - protein, 434 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRIYIWAPLAGTCGVLLLSLVITALYLLRRDQRLPPDAHKPPGGGSFRTPIQEEQADAHSTLAKIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 48 - protein, 476 aa, synthetic construct
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRIYIWAPLAGTCGVLLLSLVITALYLLRRDQRLPPDAHKPPGGGSFRTPIQEEQADAHSTLAKIKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 49 - protein, 5 aa, synthetic construct
DYVIN

SEQ ID NO: 50 - protein, 7 aa, synthetic construct
GYTFTDY

SEQ ID NO: 51 - protein, 8 aa, synthetic construct
GYTFTDYV

SEQ ID NO: 52 - protein, 17 aa, synthetic construct
EIYPGSGTNYYNEKFKA

SEQ ID NO: 53 - protein, 4 aa, synthetic construct
PGSG

SEQ ID NO: 54 - protein, 16 aa, synthetic construct
GYTFTDYVIYPGSGTN

SEQ ID NO: 55 - protein, 11 aa, synthetic construct
RGRYGLYAMDY

SEQ ID NO: 56 - protein, 9 aa, synthetic construct
GRYGLYAMD

SEQ ID NO: 57 - protein, 13 aa, synthetic construct
ARRGRYGLYAMDY

SEQ ID NO: 58 - protein, 6 aa, synthetic construct
SDYAWN

SEQ ID NO: 59 - protein, 8 aa, synthetic construct
GYSITSDY

SEQ ID NO: 60 - protein, 9 aa, synthetic construct
GYSITSDYA

SEQ ID NO: 61 - protein, 16 aa, synthetic construct
YITYSGSTSYNPSLES

SEQ ID NO: 62 - protein, 7 aa, synthetic construct
ITYSGST

SEQ ID NO: 63 - protein, 13 aa, synthetic construct
GGYYGSSWGVFAY

SEQ ID NO: 64 - protein, 11 aa, synthetic construct
GYYGSSWGVFA

SEQ ID NO: 65 - protein, 15 aa, synthetic construct
ARGGYYGSSWGVFAY

SEQ ID NO: 66 - protein, 5 aa, synthetic construct
EYTMH

SEQ ID NO: 67 - protein, 7 aa, synthetic construct
GYTFTEY

SEQ ID NO: 68 - protein, 8 aa, synthetic construct
GYTFTEYT

SEQ ID NO: 69 - protein, 17 aa, synthetic construct
GISPNIGGTSYNQKFKG

SEQ ID NO: 70 - protein, 8 aa, synthetic construct
ISPNIGGT

SEQ ID NO: 71 - protein, 7 aa, synthetic construct
RGGSFDY

SEQ ID NO: 72 - protein, 5 aa, synthetic construct
GGSFD

SEQ ID NO: 73 - protein, 9 aa, synthetic construct
ARRGGSFDY

SEQ ID NO: 74 - protein, 5 aa, synthetic construct
SFTMH

SEQ ID NO: 75 - protein, 7 aa, synthetic construct
GYTFTSF

SEQ ID NO: 76 - protein, 8 aa, synthetic construct
GYTFTSFT

SEQ ID NO: 77 - protein, 17 aa, synthetic construct
HIHPNSGISNYNEKFKG

SEQ ID NO: 78 - protein, 8 aa, synthetic construct
IHPNSGIS

SEQ ID NO: 79 - protein, 8 aa, synthetic construct
GSSRGFDY

SEQ ID NO: 80 - protein, 6 aa, synthetic construct
SSRGFD

SEQ ID NO: 81 - protein, 10 aa, synthetic construct
VRGSSRGFDY

SEQ ID NO: 82 - protein, 5 aa, synthetic construct
SSWMH

SEQ ID NO: 83 - protein, 7 aa, synthetic construct
GYTFTSS

SEQ ID NO: 84 - protein, 8 aa, synthetic construct
GYTFTSSW

SEQ ID NO: 85 - protein, 17 aa, synthetic construct
HIHPNSGISNYNEKFKG

SEQ ID NO: 86 - protein, 8 aa, synthetic construct
IHPNSGIS

SEQ ID NO: 87 - protein, 6 aa, synthetic construct
GGRFDD

SEQ ID NO: 88 - protein, 8 aa, synthetic construct
ARGGRFDD

SEQ ID NO: 89 - protein, 6 aa, synthetic construct
SDYAWN

SEQ ID NO: 90 - protein, 8 aa, synthetic construct
GYSITSDY

SEQ ID NO: 91 - protein, 9 aa, synthetic construct
GYSITSDYA

SEQ ID NO: 92 - protein, 16 aa, synthetic construct
YITYSGSTNYNPSLKS

SEQ ID NO: 93 - protein, 7 aa, synthetic construct
ITYSGST

SEQ ID NO: 94 - protein, 11 aa, synthetic construct
CWDYALYAMDC

SEQ ID NO: 95 - protein, 9 aa, synthetic construct
WDYALYAMD

SEQ ID NO: 96 - protein, 13 aa, synthetic construct
ARCWDYALYAMDC

SEQ ID NO: 97 - protein, 11 aa, synthetic construct
RASQDISNYLN

SEQ ID NO: 98 - protein, 7 aa, synthetic construct
SQDISNY

SEQ ID NO: 99 - protein, 6 aa, synthetic construct
QDISNY

SEQ ID NO: 100 - protein, 7 aa, synthetic construct
YTSRLHS

SEQ ID NO: 101 - protein, 9 aa, synthetic construct
QQGNTRPWT

SEQ ID NO: 102 - protein, 9 aa, synthetic construct
YTSGNTRPW

SEQ ID NO: 103 - protein, 12 aa, synthetic construct
YTSQQGNTRPWT

SEQ ID NO: 104 - protein, 11 aa, synthetic construct
RVSENIYSYLA

SEQ ID NO: 105 - protein, 7 aa, synthetic construct
SENIYSY

SEQ ID NO: 106 - protein, 6 aa, synthetic construct
ENIYSY

SEQ ID NO: 107 - protein, 7 aa, synthetic construct
NAKTLAE

SEQ ID NO: 108 - protein, 9 aa, synthetic construct
QHHYGTPWT

SEQ ID NO: 109 - protein, 6 aa, synthetic construct
HYGTPW

SEQ ID NO: 110 - protein, 9 aa, synthetic construct
QHHYGTPWT

SEQ ID NO: 111 - protein, 11 aa, synthetic construct
RASQSISDYLH

SEQ ID NO: 112 - protein, 7 aa, synthetic construct
SQSISDY

SEQ ID NO: 113 - protein, 6 aa, synthetic construct
QSISDY

SEQ ID NO: 114 - protein, 7 aa, synthetic construct
YASQSIS

SEQ ID NO: 115 - protein, 9 aa, synthetic construct
QNGHSFPLT

SEQ ID NO: 116 - protein, 6 aa, synthetic construct
GHSFPL

SEQ ID NO: 117 - protein, 11 aa, synthetic construct
RASENIYSNLA

SEQ ID NO: 118 - protein, 7 aa, synthetic construct
SENIYSN

SEQ ID NO: 119 - protein, 6 aa, synthetic construct
ENIYSN

SEQ ID NO: 120 - protein, 7 aa, synthetic construct
AATNLAD

SEQ ID NO: 121 - protein, 9 aa, synthetic construct
QHFWGTPRT

SEQ ID NO: 122 - protein, 6 aa, synthetic construct
FWGTPR

SEQ ID NO: 123 - protein, 11 aa, synthetic construct
RASQDIGSSLN

SEQ ID NO: 124 - protein, 7 aa, synthetic construct
SQDIGSS

SEQ ID NO: 125 - protein, 6 aa, synthetic construct
QDIGSS

SEQ ID NO: 126 - protein, 7 aa, synthetic construct
ATSSLDS

SEQ ID NO: 127 - protein, 9 aa, synthetic construct
LQYASSPWT

SEQ ID NO: 128 - protein, 7 aa, synthetic construct
YASSPWT

SEQ ID NO: 129 - protein, 9 aa, synthetic construct
LQYASSPWT

SEQ ID NO: 130 - protein, 11 aa, synthetic construct
RTSENIYSYLA

SEQ ID NO: 131 - protein, 7 aa, synthetic construct
NAKTLAE

SEQ ID NO: 132 - protein, 9 aa, synthetic construct
QHHYDTPLT

SEQ ID NO: 133 - protein, 6 aa, synthetic construct
HYDTPL

SEQ ID NO: 134 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 135 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 136 - protein, 122 aa, synthetic construct
EVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSA

SEQ ID NO: 137 - protein, 107 aa, synthetic construct
DIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 138 - protein, 116 aa, synthetic construct
EVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSS

SEQ ID NO: 139 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 140 - protein, 117 aa, synthetic construct
QVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSA

SEQ ID NO: 141 - protein, 107 aa, synthetic construct
DIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 142 - protein, 115 aa, synthetic construct
QVQLQQPGSVLVRPGASVKLSCKASGYTFTSSWMHWAKQRPGQGLEWIGHIHPNSGISNYNEKFKGKATLTVDTSSSTAYVDLSSLTSEDSAVYYCARGGRFDDWGAGTTVTVSS

SEQ ID NO: 143 - protein, 107 aa, synthetic construct
DIQMTQSPSSLSASLGERVSLTCRASQDIGSSLNWLQQEPDGTIKRLIYATSSLDSGVPKRFSGSRSGSDYSLTISSLESEDFVDYYCLQYASSPWTFGGGTKLEIK

SEQ ID NO: 144 - protein, 120 aa, synthetic construct
DVQLQESGPGLVKPSQSLSLTCTVTGYSITSDYAWNWIRQFPGNKLEWMGYITYSGSTNYNPSLKSRISITRDTSKNQFFLQLNSVTTEDTATYYCARCWDYALYAMDCWGQGTSVTVSS

SEQ ID NO: 145 - protein, 107 aa, synthetic construct
DIQMTQSPASLSASVGETVTITCRTSENIYSYLAWCQQKQGKSPQLLVYNAKTLAEGVPSRFSGSGSGTHFSLKINSLQPEDFGIYYCQHHYDTPLTFGAGTKLELK

SEQ ID NO: 146 - protein, 120 aa, synthetic construct
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSS

SEQ ID NO: 147 - protein, 120 aa, synthetic construct
QVQLVQSGAEVKKPGSSVKVSCKASGYTFTDYVINWGRQAPGQGLEWIGEIYPGSGTNYYNEKFKAKATITADKSTSTAYMELSSLRSEDTAVYFCARRGRYGLYAMDYWGQGTTVTVSS

SEQ ID NO: 148 - protein, 107 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIK

SEQ ID NO: 149 - protein, 122 aa, synthetic construct
QVQLQESGPGLVKPSQTLSLTCTVSGYSISSDYAWNWIRQPPGKGLEWIGYITYSGSTSYNPSLESRVTISRDTSKNQFSLKLSSVTAADTAVYYCARGGYYGSSWGVFAYWGQGTLVTVSS

SEQ ID NO: 150 - protein, 122 aa, synthetic construct
QVQLQESGPGLVKPSQTLSLTCTVSGYSISSDYAWNWIRQPPGKGLEWMGYITYSGSTSYNPSLESRITISRDTSKNQFSLKLSSVTAADTAVYYCARGGYYGSSWGVFAYWGQGTLVTVSS

SEQ ID NO: 151 - protein, 122 aa, synthetic construct
QVQLQESGPGLVKPSQTLSLTCTVSGYSITSDYAWNWIRQPPGKGLEWMGYITYSGSTSYNPSLESRITISRDTSKNQFSLKLSSVTAADTAVYYCARGGYYGSSWGVFAYWGQGTLVTVSS

SEQ ID NO: 152 - protein, 107 aa, synthetic construct
DIQMTQSPSSLSASVGDRVTITCRVSENIYSYLAWYQQKPGKAPKLLVYNAKTLAEGVPSRFSGSGSGTDFTLTISSLQPEDFATYYCQHHYGTPWTFGGGTKVEIK

SEQ ID NO: 153 - protein, 116 aa, synthetic construct
QVQLVQSGAEVKKPGSSVKVSCKASGYTFSEYTMHWVRQAPGQGLEWMGGISPNIGGTSYNQKFKGRVTITADKSTSTAYMELSSLRSEDTAVYYCARRGGSFDYWGQGTTVTVSS

SEQ ID NO: 154 - protein, 116 aa, synthetic construct
QVQLVQSGAEVKKPGSSVKVSCKASGYTFSEYTMHWVRQAPGQGLEWIGGISPNIGGTSYNQKFKGRATITADKSTSTAYMELSSLRSEDTAVYYCARRGGSFDYWGQGTTVTVSS

SEQ ID NO: 155 - protein, 116 aa, synthetic construct
QVQLVQSGAEVKKPGSSVKVSCKASGYTFSEYTMHWVRQAPGQGLEWIGGISPNIGGTSYNQKFKGRATLTADKSTSTAYMELSSLRSEDTAVYYCARRGGSFDYWGQGTTVTVSS

SEQ ID NO: 156 - protein, 107 aa, synthetic construct
EIVMTQSPATLSVSPGERATLSCRASQSISDYLHWYQQKPGQAPRLLIKYASQSISGIPARFSGSGSGTDFTLTISSLEPEDFAVYYCQNGHSFPLTFGQGTKLEIK

SEQ ID NO: 157 - protein, 117 aa, synthetic construct
QVQLVQSGAEVKKPGASVKVSCKASGYTFTSFTMHWVRQAPGQGLEWIGYINPSSGYTEYNQKFKDRVTITADKSTSTAYMELSSLRSEDTAVYYCVRGSSRGFDYWGQGTLVTVSS

SEQ ID NO: 158 - protein, 117 aa, synthetic construct
QVQLVQSGAEVKKPGASVKVSCKASGYTFTSFTMHWVRQAPGQGLEWIGYINPSSGYTEYNQKFKDRTTITADKSTSTAYMELSSLRSEDTAVYYCVRGSSRGFDYWGQGTLVTVSS

SEQ ID NO: 159 - protein, 117 aa, synthetic construct
QVQLVQSGAEVKKPGASVKVSCKASGYTFTSFTMHWVRQAPGQGLEWIGYINPSSGYTEYNQKFKDRTTLTADKSTSTAYMELSSLRSEDTAVYYCVRGSSRGFDYWGQGTLVTVSS

SEQ ID NO: 160 - protein, 107 aa, synthetic construct
DIQMTQSPSSLSASVGDRVTITCRASENIYSNLAWFQQKPGKAPKLLVYAATNLADGVPSRFSGSGSGTDYTLTISSLQPEDFATYYCQHFWGTPRTFGGGTKVEIK

SEQ ID NO: 161 - protein, 120 aa, synthetic construct
QVQLQESGPGLVKPSQTLSLTCTVSGGSISSDYAWNWIRQPPGKGLEWIGYITYSGSTNYNPSLKSRVTISRDTSKNQFSLKLSSVTAADTAVYYCARCWDYALYAMDCWGQGTTVTVSS

SEQ ID NO: 162 - protein, 120 aa, synthetic construct
QVQLQESGPGLVKPSQTLSLTCTVSGYSISSDYAWNWIRQPPGKGLEWIGYITYSGSTNYNPSLKSRVTISRDTSKNQFSLKLSSVTAADTAVYYCARCWDYALYAMDCWGQGTTVTVSS

SEQ ID NO: 163 - protein, 120 aa, synthetic construct
QVQLQESGPGLVKPSQTLSLTCTVSGYSISSDYAWNWIRQPPGKGLEWMGYITYSGSTNYNPSLKSRITISRDTSKNQFSLKLSSVTAADTAVYYCARCWDYALYAMDCWGQGTTVTVSS

SEQ ID NO: 164 - protein, 107 aa, synthetic construct
DIQMTQSPSSLSASVGDRVTITCRTSENIYSYLAWCQQKPGKAPKLLIYNAKTLAEGVPSRFSGSGSGTDFTLTISSLQPEDFATYYCQHHYDTPLTFGQGTKLEIK

SEQ ID NO: 165 - protein, 107 aa, synthetic construct
DIQMTQSPSSLSASVGDRVTITCRTSENIYSYLAWCQQKPGKAPKLLVYNAKTLAEGVPSRFSGSGSGTDFTLTISSLQPEDFATYYCQHHYDTPLTFGQGTKLEIK

SEQ ID NO: 166 - protein, 120 aa, synthetic construct [sequence omitted]

SEQ ID NO: 167 - protein, 107 aa, synthetic construct [sequence omitted]

SEQ ID NO: 168 - protein, 120 aa, synthetic construct
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 169 - protein, 107 aa, synthetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 170 - protein, 124 aa, synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 171 - protein, 111 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 172 - protein, 121 aa, synthetic construct [sequence omitted]

SEQ ID NO: 173 - protein, 106 aa, synthetic construct [sequence omitted]

SEQ ID NO: 174 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 175 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 176 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 177 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 178 - protein, 119 aa, synthetic construct [sequence omitted]

SEQ ID NO: 179 - protein, 107 aa, synthetic construct [sequence omitted]

SEQ ID NO: 180 - protein, 123 aa, synthetic construct [sequence omitted]

SEQ ID NO: 181 - protein, 111 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQI

SEQ ID NO: 182 - protein, 121 aa, synthetic construct [sequence omitted]

SEQ ID NO: 183 - protein, 107 aa, synthetic construct [sequence omitted]

SEQ ID NO: 184 - protein, 116 aa, synthetic construct
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 185 - protein, 107 aa, synthetic construct
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 186 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 187 - protein, 107 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 188 - protein, 121 aa, synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 189 - protein, 107 aa, synthetic construct
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIK

SEQ ID NO: 190 - protein, 13 aa, synthetic construct
THTCPPCPAPELL

SEQ ID NO: 191 - protein, 15 aa, synthetic construct
NASSMCVPSPAPELL

SEQ ID NO: 192 - protein, 6 aa, synthetic construct
HHHHHH

SEQ ID NO: 193 - protein, 7 aa, synthetic construct
DYKDDDD

SEQ ID NO: 194 - protein, 8 aa, synthetic construct
DYKDDDDK

SEQ ID NO: 195 - protein, 7 aa, synthetic construct
DYKDDDK

SEQ ID NO: 196 - protein, 8 aa, synthetic construct
DLYDDDDK

SEQ ID NO: 197 - protein, 15 aa, synthetic construct
GLNDIFEAQKIEWHE

SEQ ID NO: 198 - protein, 26 aa, synthetic construct
KRRWKKNFIAVSAANRFKKISSSGAL

SEQ ID NO: 199 - protein, 6 aa, synthetic construct
EEEEEE

SEQ ID NO: 200 - protein, 9 aa, synthetic construct
YPYDVPDYA

SEQ ID NO: 201 - protein, 9 aa, synthetic construct
YAYDVPDYA

SEQ ID NO: 202 - protein, 9 aa, synthetic construct
YDVPDYASL

SEQ ID NO: 203 - protein, 10 aa, synthetic construct
EQKLISEEDL

SEQ ID NO: 204 - protein, 8 aa, synthetic construct
WRHPQFGG

SEQ ID NO: 205 - protein, 8 aa, synthetic construct
WSHPQFEK

SEQ ID NO: 206 - protein, 13 aa, synthetic construct
SLAELLNAGLGGS

SEQ ID NO: 207 - protein, 8 aa, synthetic construct
TQDPSRVG

SEQ ID NO: 208 - protein, 14 aa, synthetic construct
GKPIPNPLLGLDST

SEQ ID NO: 209 - protein, 461 aa, synthetic construct
MSVPTQVLGLLLLWLTDARCEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 701 aa, synthetic construct
MEWSWVFLFFLSVTTGVHSEVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 377 aa, synthetic construct
MSVPTQVLGLLLLWLTDARCDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHSGSSSSGSSSSGSSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 212 - protein, 461 aa, synthetic construct
MSVPTQVLGLLLLWLTDARCEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 701 aa, synthetic construct
MEWSWVFLFFLSVTTGVHSEVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 861 aa, synthetic construct
MEWSWVFLFFLSVTTGVHSEVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSVEGGSGGSGGSGGSGGVDDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKGSSSSGSSSSGSSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 215 - protein, 446 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 679 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 215 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 218 - protein, 377 aa, synthetic construct
MSVPTQVLGLLLLWLTDARCDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHSGSSSSGSSSSGSSSSTKKTQLQLEHLLLDLQMILNGINNYKNPKLTAMLAKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 219 - protein, 377 aa, synthetic construct
MSVPTQVLGLLLLWLTDARCDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHSGSSSSGSSSSGSSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 220 - protein, 446 aa, synthetic construct; identical to SEQ ID NO: 215

SEQ ID NO: 221 - protein, 678 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLVQSGAEVKKSGESLKISCKGSGYSFTSYWIGWVRQMPGKGLEWMGIFYPGDSSTRYSPSFQGQVTISADKSVNTAYLQWSSLKASDTAMYYCARRRNWGNAFDIWGQGTMVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 364 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSTWTFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHSGSSSSGSSSSGSSSSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 223 - protein, 446 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 679 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 357 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHSGSSSSGSSSSGSSSSTKKTQLQLEHLLLDLQMILNGINNYKNPKLTAMLAKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 226 - protein, 441 aa, synthetic construct
EIVLTQSPATLSLSPGERATLSCRASQSVRSYLAWYQQKPGQAPRLLFSDASNRATGIPARFSGSGSGTDFTLTISSLEPEDFAVYYCQQYRYSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 677 aa, synthetic construct
EVQLVESGGGLVQPGRSLRLSCAASGFTFDNYAMHWVRQAPGKGLEWVSGISRSSGDIDYADSVKGRFTISRDNAKNSLYLQMNSLRAEDTALYYCARGGVGSFDTWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 358 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHSGSSSSGSSSSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 229 - protein, 441 aa, synthetic construct
EIVLTQSPATLSLSPGERATLSCRASQSVRSYLAWYQQKPGQAPRLLFSDASNRATGIPARFSGSGSGTDFTLTISSLEPEDFAVYYCQQYRYSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 676 aa, synthetic construct
EVQLVESGGGLVQPGRSLRLSCAASGFTFDNYAMHWVRQAPGKGLEWVSGISRSSGDIDYADSVKGRFTISRDNAKNSLYLQMNSLRAEDTALYYCARGGVGSFDTWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLVQSGAEVKKSGESLKISCKGSGYSFTSYWIGWVRQMPGKGLEWMGIFYPGDSSTRYSPSFQGQVTISADKSVNTAYLQWSSLKASDTAMYYCARRRNWGNAFDIWGQGTMVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - protein, 359 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSTWTFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHSGSSSSGSSSSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 232 - protein, 126 aa, synthetic construct
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVKP

SEQ ID NO: 233 - protein, 126 aa, synthetic construct
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVRP

SEQ ID NO: 234 - protein, 446 aa, synthetic construct; identical to SEQ ID NO: 215

SEQ ID NO: 235 - protein, 680 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 236 - protein, 214 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 441 aa, synthetic construct; identical to SEQ ID NO: 226

SEQ ID NO: 238 - protein, 678 aa, synthetic construct
EVQLVESGGGLVQPGRSLRLSCAASGFTFDNYAMHWVRQAPGKGLEWVSGISRSSGDIDYADSVKGRFTISRDNAKNSLYLQMNSLRAEDTALYYCARGGVGSFDTWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 239 - protein, 214 aa, synthetic construct; identical to SEQ ID NO: 236